(19) United States
(12) Patent Application Publication
(10) Pub. No.: US 2024/0066061 A1
(43) Pub. Date: Feb. 29, 2024

(54) A CXCR3+ CELL OR CELL PREPARATION FOR USE IN CANCER TREATMENT

(71) Applicant: Charité-Universitätsmedizin Berlin, Berlin (DE)

(72) Inventors: Hans Dieter VOLK, Berlin (DE); Michael SCHMÜCK-HENNERESSE, Berlin (DE); Tino VOLLMER, Berlin (DE); Petra REINKE, Berlin (DE); Stephan SCHLICKEISER, Berlin (DE)

(73) Assignee: CHARITÉ-UNIVERSITÄTSMEDIZIN BERLIN, Berlin (DE)

(21) Appl. No.: 18/261,121

(22) PCT Filed: Jan. 12, 2022
(86) PCT No.: PCT/EP2022/050571

§ 371 Date: Jul. 12, 2023

(30) Foreign Application Priority Data
Jan. 12, 2021 (EP) 21151232.2
Jan. 12, 2021 (EP) 21151233.0
Jan. 13, 2021 (EP) 21151438.5
Jan. 13, 2021 (EP) 21151447.6

(51) Int. Cl. A61K 35/17; A61K 39/00; A61P 35/00; C07K 14/715

(52) U.S. Cl. CPC A61K 35/17; A61K 39/4611; A61K 39/4631; A61K 39/464421; A61P 35/00; C07K 14/7158; A61K 2239/15; A61K 2239/21; A61K 2239/22

(57) ABSTRACT

The invention provides a modified T cell, or an isolated population of immune cells expressing a CXCR3 isoform selected from CXCR3A, CXCR3B, and CXCR3alt, and optionally, further expressing transgenes comprising an artificial T cell receptor, and/or a CXCR3 ligand, for use as a medicament. The invention also provides the methods to obtain said cells, or populations of cells from a plurality of immune cells derived from a human subject. The invention also relates to assessment of CXCR3 splice variants and its ligands CXCL9, CXCL10, and CXCL11 in muscle-invasive bladder cancer (MIBC) patients, to enable patients to be stratified for their predicted response to a chemotherapy drug treatment, or clinical outcome.

The present invention relates to transgenic expression of the chemokine receptor CXCR3 and its ligands in human T cells, thereby enhancing their efficacy and survival to provide an improved cell transfer medicament in patients suffering from cancer, chronic viral infection or autoimmunity.

This application claims the benefit of priority of European patent applications EP21151233.0 and EP21151232.2, both filed 12 Jan. 2021; and EP21151438.5 and EP21151447.6, both filed 13 Jan. 2021, all of which are incorporated herein by reference.

### BACKGROUND OF THE INVENTION

Bladder cancer (BC) ranks among the 10 most frequent malignancies in Europe and the U.S.A. Tumour eradicating T cell responses can be induced via Bacillus Calmette-Guérin (BCG)-therapy in limited, non-muscle-invasive bladder cancer (NMIBC), or sometimes via PD-1/PD-L1-blockade in progressed, muscle-invasive bladder cancer (MIBC). In the current standard-of-care treatment, MIBC-patients receive neoadjuvant chemotherapy (NAC) prior to radical cystectomy (RC), which improves overall survival (OS) compared to RC alone (Vale C. Lancet (2003) 361: 1927-1934). Between 25-40% of MIBC patients respond to NAC, as defined by downstaging of the pathology grade of the tumour. Non-responder MIBC-patients do not exhibit tumour reduction, and remain in a muscle-invasive disease state until the RC is initiated. It is therefore vital to effectively identify cancer patients who are most likely to benefit from neoadjuvant drug treatment. Novel stratification systems for predicting the response to NAC are needed to improve the treatment protocols for the clinical benefit of the non-responder MIBC-patients. The benefits of neoadjuvant treatment have also been proven for locally advanced breast cancer (Eltahir A. et al. Am. J. Surg. 1998: 175(2):127-32), gastric cancer (Cunningham D. et al. N. Engl. J. Med. 2006: 355(1): 11-20) and oesophageal cancer (van Hagen P. N. Engl. J. Med. 2012: 366(22):2074-84).

A robust CD8⁺ T cell response against malignant cells can be induced by chemotherapy, and other types of cancer treatment (Galluzzi, et al. Cancer Cell (2015), 28: 690-714). The chemokine receptor CXCR3 (UniProt P49682), which can be engaged by the IFN-gamma-inducible ligands CXCL9 (MIG, UniProt Q07325), CXCL10 (IP-10, UniProt P02778), and CXCL11 (I-TAC, UniProt 014625), is heterogeneously expressed within the CD8⁺ T cell compartment. CXCR3 mediates the homing and positioning of CD8⁺ T cells in the secondary lymphoid compartment, but also to peripheral inflammation and malignant tissues. The activity of the intra-tumoural CXCR3-chemokine system is required for directing CD8⁺ T cell responses, and the efficacy of anti-PD-1 inhibition in mice. The CXCR3-ligand axis can be activated via chemotherapeutic agents in pre-clinical models supporting anti-tumour efficacy.

In contrast to a single transcript of CXCR3 in the mouse genome, humans express three CXCR3 isoforms, the main isoform CXCR3A, and two splice-variants CXCR3B and CXCR3alt (Ehlert J. et al. J. Immunol. (2004) 172:6234-6240). The CXCR3-isoforms exhibit different affinities for the CXCR3-ligands (CXCL9,10,11) and selectively activate distinct signalling pathways upon binding of different ligands (Metzemaekers M. Front. Immunol. (2018) 8:1970).

### SUMMARY OF THE INVENTION

The immune compartment in tumours of chemotherapy-treated patients has predominantly been studied post-treatment, and the identified immune markers are rarely tested for their functional anti-tumour potency. In the examples presented here, the inventors stratify the intra-tumoural activity of the CXCR3-chemokine system in a cohort of BC patients, in relation to their response to NAC. The inventors show that the chemokine CXCL11 and its specific receptors CXCR3alt and CXCR3A are powerful predictors of the response of MIBC patients to NAC, and dissect the functional relevance of CXCR3 ligand signals through each receptor in determining T cell potency in response to cancer or viral antigens.

A first aspect of the invention is a T cell, expressing a CXCR3 variant selected from CXCR3A, CXCR3B, and/or CXCR3alt from a transgene, particularly when the CXCR3 variant, or one of the variants is CXCR3A or CXCR3alt. In particular embodiments, the modified T cell is a CD3+ CD8⁺ T cell. In certain embodiments, the CXCR3 variant transgene comprises the reverse complement of the premRNA, or coding mRNA transcript for CXCR3A, CXCR3B or CXCR3alt, or a sequence encoding an amino acid sequence 95% similar that encoded by the above sequences, retaining the biological function of the CXCR3 variant protein. In some embodiments, the expression of CXCR3A, and/or CXCR3alt is higher than that of CXCR3B, particularly with an expression ratio over 1.

In optional embodiments, the modified T cell additionally expresses a recombinant chimeric antigen receptor (CAR), or transgenic T cell receptor (TgTCR), recognising a cancer, pathogen-derived, or tissue specific antigen. In further embodiments, the modified T cell also expresses one or more CXCR3 ligand transgenes, comprising the reverse complement of the premRNA, or coding transcript of CXCL9, CXCL10, and/or CXCL11.

A second aspect of the invention is an isolated preparation of immune cells, particularly of T cells, of which at least (≥) 50%, particularly 70%, more particularly ≥8.0% are positive for CXCR3A, CXCR3B and/or CXCR3alt expression. In some embodiments the isolated preparation of cells is derived from a cancer patient sample, or comprises cells expressing a CXCR3 variant according to the first aspect of the invention, or conversely, no transgenes.

In particular embodiments, the modified T cell, or the isolated populated of immune cells expressing CXCR3 variants has a chemotactic index over 1 when stimulated with CXCR3 ligands, or proliferates, has enhanced lytic potential, or makes more effector cytokines, compared to unmodified, or unfractionated control immune cells.

A third aspect of the modified T cell, or the isolated preparation of cells according to the above aspects of the invention, provides their use as a medicament, particularly to improve T cell immunity, suppress infection or inflammation, or more particularly to treat solid forms of cancer.

A fourth aspect provided by the invention is a method to isolate CXCR3+ cells from human leukocyte samples. A related aspect is a method to obtain a preparation of CXCR3+ cells according to the invention, which comprises providing a plurality of human immune cells, and inserting a transgene, or transgenes encoding a sequence selected from SEQ ID NO 001 to SEQ ID 006, and optionally, a further sequence selected from SEQ ID NO 007 to SEQ ID NO 015. A final embodiment of the invention is a method to expand or activate an isolated preparation of cells obtained according to the methods provided above, by culturing cells with CXCL9, CXCL10 and/or CXCL11.

### DETAILED DESCRIPTION OF THE INVENTION

### Terms and Definitions

For purposes of interpreting this specification, the following definitions will apply, and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The terms gene expression or expression, or alternatively the term gene product, may refer to either of, or both of, the processes—and products thereof—of generation of nucleic acids (RNA) or the generation of a peptide or polypeptide, also referred to transcription and translation, respectively, or any of the intermediate processes that regulate the processing of genetic information to yield polypeptide products.

The term gene expression may also be applied to the transcription and processing of an RNA gene product, for example a regulatory RNA or a structural (e.g. ribosomal) RNA. If an expressed polynucleotide is derived from genomic DNA, expression may include splicing of the mRNA in a eukaryotic cell. Expression may be assayed both on the level of transcription and translation, in other words mRNA and/or protein product. The inventors show both methods of CXCL11, CXCL9 and CXCL10 measurement were useful for prediction of OS in chemotherapy-receiving MBIC in data provided in the examples. For the expression of CXCR3 ligand CXCL11 in a sample, 22.4 pg per 10 mg of tissue as measured by an ELISA system such as Luminex is a useful threshold for positive expression according to the invention. CXCR3 isoforms can be assessed at the level of mRNA expression, but may be measured at the level of surface protein expression with ligands which distinguish between the variants. FIG. 4f demonstrates thresholds for CXCR3A, CXCR3B, and CXCR3alt expression as measured by realtime qPCR using the assay provided in the section entitled Intra-tumoural analysis of mRNA CXCR3-variants, whereby the sample is considered positive if the CXCR3 variant is expressed >0.1 times, particularly >0.2 times as much as the indicated house-keeping genes.

The term splice variant or isoform refers here to three polypeptides derived from different splicing arrangements of the CXCR3 gene. CXCR3A refers to the canonical receptor (Loetscher et al. J. Exp. Med. 1996 184:963-969), the NCBI reference DNA sequence encoding CXCR3A is provided in SEQ ID NO 001. Two naturally occurring CXCR3 splice variants have been identified in humans. One alternative splicing of the CXCR3 genes is the CXCR3B mRNA (NCBI reference DNA SEQ ID NO 002), producing a polypeptide characterised by an additional N-terminal 51 amino acids (Lasagni et al. J. Exp. Med. 2003 197:1537-1549). The alternative, or CXCR3alt, splice variant mRNA product is truncated (NCBI reference DNA SEQ ID NO 003, Ehlert et al. 2004), producing a polypeptide lacking the 6^{th} and 7^{th} transmembrane helices, and the third intracellular loop, leading to a short cytoplasmic C-terminus. The relative abundance of these splice variants may be measured by means of variant-specific primers or molecular probes such as those used in the examples.

In the context of the present specification, the terms sequence identity and percentage of sequence identity refer to a single quantitative parameter representing the result of a sequence comparison determined by comparing two aligned sequences position by position. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively. Reference to identical sequences without specification of a percentage value implies 100% identical sequences (i.e. the same sequence).

As used herein, the term treating or treatment of any disease or disorder (e.g. cancer) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease are generally known in the art, unless specifically described hereinbelow.

A human T cell according to the invention encompasses both αβ and γδ cell receptors (TCR) expressing cells (expressing CD3, and particularly CD4, or CD8), and natural killer (NK) cells and NK T cells (expressing CD56). These cells can also be characterised by the absence of cell surface markers which characterise myeloid cells, B cells, innate lymphoid cells, endothelial, stromal, or epithelial cells, neurons, erythrocytes, or fibroblasts.

The term neoadjuvant treatment in the context of the present specification relates to pharmaceutical formulations comprising one or more antineoplastic drugs. In the case of bladder cancer, the antineoplastic drugs are most commonly chemotherapy with platinum drug-based treatments, but can also include Bacillus Calmette-Guerin, or cancer immunomodulatory therapy. The neoadjuvant treatment regime may additionally include radiation treatment of the tumour.

The standard of care regime of neoadjuvant chemotherapy is a formulation of the drugs methotrexate, vinblastine, doxorubicin or epirubucin, and cisplatin, but may comprise similar drugs, for example, paclitaxel, carboplatin, adriamycin, gemcitabine, filgramastim, pemetrexed, vinorelbine, oxaliplatin, vinflunine, or doxetaxel.

In the context of the present specification, the term cancer immunotherapy, biological or immunomodulatory therapy is meant to encompass types of cancer treatment that help the immune system to fight cancer. Non-limiting examples of cancer immunotherapy include immune checkpoint inhibitors and agonists, T cell transfer therapy, cytokines and their recombinant derivatives, adjuvants, and vaccination with small molecules or cells.

In the context of the present specification, the term checkpoint inhibitory agent or checkpoint inhibitory antibody is meant to encompass an agent, particularly an antibody (or antibody-like molecule) capable of disrupting an inhibitory signalling cascade that limits immune cell activation, known in the art as an immune checkpoint mechanism. In certain embodiments, the checkpoint inhibitory agent or checkpoint inhibitory antibody is an antibody to CTLA-4, PD-1, PD-L1, B7H3, VISTA, TIGIT, TIM-3, CD158, or TGF-beta.

In certain embodiments, the immune checkpoint inhibitor agent is selected from the clinically available antibody drugs ipilimumab (Bristol-Myers Squibb; CAS No. 477202-00-9), nivolumab (Bristol-Myers Squibb; CAS No 946414-94-4), pembrolizumab (Merck Inc.; CAS No. 1374853-91-4), pidilizumab (CAS No. 1036730-42-3), atezolizumab (Roche AG; CAS No. 1380723-44-3), avelumab (Merck KGaA; CAS No. 1537032-82-8), durvalumab (Astra Zenaca, CAS No. 1428935-60-7), and cemiplimab (Sanofi Aventis; CAS No. 1801342-60-8).

In the context of the present specification, the term checkpoint agonist agent or checkpoint agonist antibody is meant to encompass an agent, particularly but not limited to, an antibody (or antibody-like molecule) capable of enhancing an immune cell activation signalling cascade. The term checkpoint agonist agent further encompasses cytokines, vaccines, adjuvants and agonist antibodies that promote immune activation. Non-limiting examples of cytokines known to stimulate immune cell activation include, IL-12, IL-2, IL-15, IL-21 and interferon-alpha. In certain embodiments, the checkpoint agonist agent or checkpoint agonist antibody is an antibody to CD122, CD137, ICOS, OX40, or CD40.

In certain embodiments, the immune checkpoint agonist agent is selected from the clinically available drugs aldesleukin (Novartis, Cas. No 110942-02-4), interferon alfa-2b (Merck, CAS No. 215647-85-1), imiquimod (Apotex, CAS No. 99011-02-6), PF-8600 (Pfizer), Poly ICLC (Oncovir, CAS No. 59789-29-6), Cabiralizumab (Apexigen, 1613144-80-1) or utomilumab, (CAS No. 1417318-27-4).

The term cycle threshold or CT in the context of the present specification relates to a quantitative nucleic acid measurement, for example a measurement made with a quantitative polymerase chain reactions (qPCR). This method involves repeated cycles of nucleic acid amplification using nucleic acid probes which hybridise the target biomarker, to generate a product emitting a fluorescent signal, which can be measured to determine the amount of starting genetic material. The cycle threshold may be an average value, or the average value of a number of replicate samples. Other quantitative measurements may substitute the cycle threshold, such as a crossing point, or an adjusted inflexion point.

As used herein, the terms pharmaceutical composition or pharmaceutical formulation refers to a compound of the invention, or a pharmaceutically acceptable salt thereof, together with at least one pharmaceutically acceptable carrier. In certain embodiments, the pharmaceutical composition according to the invention is provided in a form suitable for topical, parenteral or injectable administration.

The terms response or drug responder used here, particularly in regards to the antineoplastic treatment of BC, refers to tumour down-grading as measured by a pathology analysis. The term further encompasses positive clinical outcomes and the presence of immune infiltrate in the tumour, in conditions where a patient's response to treatment can also be linked both to overall survival, and the number of T cells present in the tumour.

The T cell activity state of a sample, is a measure of the potency of T cells in a tissue sample, reflecting their local numbers, activation phenotype, and chemokine milieu. The data presented in the examples demonstrates that the expression levels of the CXCR3 isoforms CXCR3A and CXCR3alt and their ligands, CXCL9, CXCL19 and CXCL11, are directly correlated with the T cell markers CD3, and CD8, and better OS. The presence of CXCR3B and CXCL4 however, correlate with poor OS. Phenotypic analysis shows that CD8+ CXCR3+ stem cell memory cells in healthy and patient lymphocyte samples respond to antigen and CXCR3 ligands cues by migration, proliferation, and production of cytokines. These correlations can be used to quantitatively assess the likelihood of a cancer patient responding favourably to an antineoplastic treatment which relies on T cell activation as one of its mechanisms of action, and is thus associated with overall survival following treatment. The T cell activity state therefore, is a combined measure of the status of T cells present in the sample with respect to the expression of CXCR3+ isoforms and its ligands, in other words the presence of CXCR3A+ and CXCR3alt+ antigen-experienced T cells, and a CXCL9, CXCL10, CXCL11 rich environment, together confer a high potential for local T proliferation and cytokine production. CXCR3 expression is observed on a variety of immune subsets which contribute to tumour infection and immunity, and autoimmunity, including natural killer cells, B cells, and macrophages. The skilled artisan will understand the presence of other immune cells may additionally contribute to the T cell activity state.

A CXCR3+ Cell or Isolated Population of CXCR3+ Cells for Medical Use

A first aspect of the invention is a T cell expressing a CXCR3 variant selected from CXCR3A, CXCR3B, and/or CXCR3alt from an artificially inserted transgene, particularly when the CXCR3 variant includes CXCR3A or CXCR3alt. This can be a cell which does, or does not, natively express CXCR3, particularly a CD4⁺ or CD8⁺ T cell, or an NK or NKT cell.

In particular embodiments, depending on the immune modulating effect desired in the end product, the modified immune cell is a CD3⁺ T cell, selected from, a CD4⁺ T helper cell or CD8⁺ T cell (expressing a CD3⁺, αβ or γδ TCR⁺, and/or a lineage specific transcription marker such as GATA3, Tbet, or Eomes), or a T regulatory (Treg) cell (expressing CD4, CD25, and the transcription marker forkhead box P3 (foxp3)), more particularly a CD3⁺ CD8⁺ T cell. The invention provides a potent T cell product which may deliver diverse immune modulating signals, by conferring enhanced CXCR3-dependent activation and proliferation on either, for example, a suppressive cell (for example a GATA3⁺ CD4⁺ T cell, or foxp3+Treg), or an inflammatory cell CD8⁺ CD45RO⁺ memory cell or a CD56⁺ NKT cell.

The data in the examples shows that the CXCR3 variants CXCR3A and CXCR3alt are directly correlated with increased numbers and/or a greater proportion of T cell markers in tumours, which are in turn correlated with better clinical outcomes of BC patients following neoadjuvant treatment providing broad antigenic stimulation of tumour-specific T cells. The differing relationship between each CXCR3 variant, CD3 T cells, and patient outcomes suggests that isoforms have specific downstream effects, which may be harnessed for medical in different settings use by transgenic manipulation of the CXCR3 expression profile. Stimulation of CXCR3^{high} stem cell and central memory cells with CXCR3 ligands in particular is demonstrated to lead to improved proliferation, migration and cytokine secretion of tumour-specific and virus-specific CD8+ T cells in vitro. The inventors propose that the transgenic expression of CXCR3 variants, particularly CXCR3A and CXCR3alt which were associated strongly with the presence and potency of T cell responses, will confer improved proliferation, migration and cytokine secretion on a recipient immune cell, particularly a CD3⁺ T cell.

In certain embodiments, the modified cell expresses a CXCR3 variant transgene comprising: CXCR3alt, CXCR3B, or particularly CXCR3A only.

In certain embodiments, the modified cell expresses a transgene, or two transgenes comprising: CXCR3A and CXCR3B, CXCR3alt and CXCR3B, or particularly, CXCR3A and CXCR3alt together.

In certain embodiments, the modified cell expresses a transgene, or several transgenes encoding the CXCR3alt, CXCR3A and CXCR3B proteins.

In order to confer the desired functional phenotype, it is understood that these CXCR3 variants should be expressed as a protein present at the cell surface. The CXCR3 variant gene sequences provided here are provisional based on the present knowledge of these newly described isoforms, and specific amino acids, or nucleic acids, may change in the future as data from more human subjects reveals new alleles. The transgenes generally encompass different isoforms of the CXCR3 protein of UniProt P49682.

The following embodiments of the invention address the composition of the CXCR3 transgene, which may either be transiently, or stably incorporated into the cell. In some embodiments, the CXCR3 variant transgene, or transgenes, comprise the reverse complement of the premRNA transcript, containing both introns and exons of the CXCR3 variants CXCR3A, CXCR3B, or CXCR3alt, particularly a sequence selected from SEQ ID NO 001, SEQ ID NO 002 and/or SEQ ID NO 003. In alternative embodiments, the CXCR3 transgene, or transgenes comprise the reverse complement of the coding mRNA transcript, that has only the exons of the CXCR3 variant genes, particularly a sequence selected from SEQ ID NO 004, SEQ ID NO 005, and/or SEQ ID NO 006. In another embodiment, the CXCR3 transgene comprises a sequence≥95% identical to the amino acid sequence encoded by SEQ ID NO 001, SEQ ID NO 002, SEQ ID NO 003, SEQ ID NO 004, SEQ ID NO 005 and/or SEQ ID NO 006, where the encoded protein has the same biological activity as SEQ ID NO 001, SEQ ID NO 002, SEQ ID NO 003, SEQ ID NO 004, SEQ ID NO 005 and/or SEQ ID NO 006, respectively, particularly if the CXCR3 transgene encodes an amino acid sequence that has ≥96%, ≥97, ≥98 or even ≥99% sequence identity to the amino acid sequence encoded by SEQ ID NO 001, SEQ ID NO 002, SEQ ID NO 003, SEQ ID NO 004, SEQ ID NO 005 and/or SEQ ID NO 006.

The biological activity conferred by functional CXCR3 transgene expression can be assessed by several different means by comparing a cell, or a population of cells bearing the transgene or transgenes with an unmodified, control cell or population. The first functional assay for CXCR3 provided, is the ability to migrate towards a stimuli of 100 ng/ml CXCL9, CXCL10, or CXCL11 as in FIG. 3a, and FIG. 3b. CXCR3 also confers enhanced proliferation in response to combined stimulus with antigen and CXCL11, for example using an assay measuring CFSE dilution such as that used in FIG. 4e.

The data in the examples shows the functionality of CXCR3 expression by human T cells from healthy volunteers or cancer patients mediates migration with a chemotactic index over 2 towards CXCL9, CXCL10, or CXCL11 stimulation (FIGS. 3a and b), or up to 10⁴ fold enrichment of CXCR3+ cells after 21 days simulation with CMV epitopes and CXCL11, as well as increased intracellular production of IFNγ and TNFα in response to tumour antigens compared to unstimulated cells (FIGS. 4a and c). The assays listed reflect the desired qualities of enhanced effector efficacy, and post-engraftment survival, which are desirable for the modified cell for use as an immune status-modulating medicament.

The genetic engineering method by which the transgene or transgenes according to the invention is inserted into the cell is not particularly limited, and may be selected from, but not limited to, the following list of technical approaches:
1. Random integration, wherein the transgene is delivered into the cell by a:
   a. a lentivirus, particularly a 3^{rd} generation lentivirus (Dull et al. J. Virol. 1998, 72(11):8463);
   b. a gamma-retrovirus, particularly a T cell transduction with a gamma-retrovirus (Rossig et al. Blood 2002, 99(6):2009);
   c. transposon-transposase based approach, particularly a PiggyBac transposon (Nakazawa et al. J. Immunother. 2009, 32(8):828), or a Sleeping Beauty (TC1-like) transposon system (Ivics et al. Cell 1997, 91(4):501; (Huang et al. Blood 2006, 107(2):483);
2. Targeted integration, comprising targeted transgene integration using a homology directed repair (HDR)-mediated or a nuclease assisted system comprising:
   a. a template format, for example a template format selected from a single stranded or double stranded DNA system (Roth et al. Nature 2018, 559(7714):405), or an adeno-associated virus (AAV) integration site, such as, for example, AAV serotype 6 (Dever et al. Nature 2016, 539(7629):384; Eyquem et al. Nature 2017, 543(7643):113), or a non-integrating lentivirus (Lombard et al. Nat. Biotechnol. 2007, 25(11):1298);
   b. a nuclease, for example a Zinc-finger nuclease, transcription activator-like (TAL) effector nuclease, a mega-TAL, clustered regularly interspaced short palindromic repeats (CRISPR)-Cas;
   c. a nuclease-free system;
3. Transient transgene delivery of mRNA using:
   a. in vitro electroporation (Schaft et al. Cancer Immunol. 2005, 55:1132);
   b. a nanoparticle;
   c. a virus.

In certain embodiments, the modified immune cell according to the invention expresses more CXCR3A and/or CXCR3alt, compared to CXCR3B, particularly wherein the ratio of CXCR3A, CXCR3alt, or combined CXCR3A and CXCR3alt expression compared to CXCR3B expression is greater than 1. CXCR3 variants may be measured at the protein level or the nucleic acid level, as shown in FIG. 4f. The data in the examples shows that the expression ratio of CXCR3A and CXCR3alt expression compared to CXCR3B expression is more than 1 on stem cell memory CD8⁺ T cells, which show enhanced responsiveness to stimulation with CXCR3 ligands in vitro.

In further embodiments of the modified T cell expressing the CXCR3 variant transgene or transgenes according to the first aspect of the invention, the cell expresses an additional transgenic protein. In some embodiments the additional transgenic protein is a recombinant chimeric antigen receptor protein comprising the following essential structural components:
a. a signal peptide,
b. a target specific recognition domain, particularly wherein the target is selected from a tumour-associated surface antigen, a lineage-specific antigen, a tissue-specific surface antigen, or a virus-specific surface antigen,
c. an effector domain comprising a transmembrane region and one or more intracellular signalling domains, particularly a CD3 zeta signalling domain, and
d. a linker region, connecting domain (b) and domain (c).

In another embodiment, the additional transgene expressed by the modified T cell is a transgenic T cell receptor (TgTCR), wherein the TgTCR recognises an immunotherapy target selected from a tumour-associated surface antigen, a lineage-specific antigen, a tissue-specific surface antigen, or a virus-specific surface antigen.

The CAR target specific recognition domain, or the TgTCR from the above embodiments, may recognise, in other words, display specific binding towards, an epitope from an molecule selected from, but not limited to, LMP1 (Epstein-Barr virus), CMV (cytomegalovirus), GD2, L1CAM (neuroblastoma), Her2 (colon, sarcoma, glioblastoma, bladder), IL13Ra2, EGFRvIII (glioblastoma), CD133 (HCC, pancreatic and colorectal), mesothelin (pancreatic), CAIX (renal), CEACAM5 (gastrointestinal), TAG-72, CEA (colon), COA-1 (colorectal), PSMA (prostate), or c-MET (breast). These antigens are viral, or tissue antigens, or antigens upregulated in tumour cells, conferring tissue, or disease specific activation signals, which in concert with increased CXCR3-mediated migration towards inflammation characterised by CXCR3 ligands, target T cell activity to prescribed physiological sites (Li et al. Signal Trans. And Targeted Ther. 2019, 4:35).

In another embodiment of modified T cell according to the invention, the cell expresses a CXCR3 ligand transgene encoding a recombinant protein comprising a human CXCR3 ligand, in order to confer an autocrine activation signal to the transgenic CXCR3 receptor. In one embodiment, the CXCR3 ligand transgene comprises both a CXCR3 ligand transgene promotor sequence, and one of the following CXCR3 ligand sequence variants:
a. the reverse complement of a premRNA transcript (both introns and exons) of CXCL9, CXCL10, and/or CXCL11, particularly a sequence selected from SEQ ID NO 007, SEQ ID NO 008, SEQ ID NO 009, SEQ ID NO 010 and/or SEQ ID NO 011, or
b. the reverse complement of a coding mRNA transcript (exons only) of CXCL9, CXCL10, and/or CXCL11, particularly a sequence selected from SEQ ID NO 012, SEQ ID NO 013, SEQ ID NO 014 and/or SEQ ID NO 015, or
c. nucleic acids encoding an amino acid sequence that has at least (≥) 95% sequence identity to the amino acid sequence encoded by SEQ ID NO 007, SEQ ID NO 008, SEQ ID NO 009, SEQ ID NO 010, SEQ ID NO 011, SEQ ID NO 012, SEQ ID NO 013, SEQ ID NO 014 and/or SEQ ID NO 015, particularly where the encoded protein has the same biological activity as the amino acid sequence encoded by SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015, respectively. In more particular embodiments, the CXCR3 transgene encodes an amino acid sequence that has ≥96%, ≥97, ≥98 or even ≥99% sequence identity to the amino acid sequence encoded by SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015.

In certain embodiments, the CXCR3 ligand transgene promoter is a constitutive promotor, such as the CMV immediate early promoter. In other embodiments, the promoter is conditionally expressed, for example, activated by T cell receptor ligation to allow inducible expression of the CXCR3 ligand transgene downstream of native or transgenic antigen receptor ligation, in order to limit non-specific activation and harmful inflammatory side-effects. This might be achieved by inclusion of antigen response elements such as ARRE-1 or ARRE-2, or a CD28 response region. It is understood that for the CXCR3 ligand to confer a functional advantage on the modified T cell, the ligand must be secreted in order to signal through CXCR3.

SEQ NO ID 007 to SEQ NO ID 015 provide splicing variants of the CXCL9, CXCL10 and CXCL11 genes currently known in the art. The application of commercially available CXCL9, CXCL10 and CXCL11 at a concentration of 10 ng/ml to CXCR3 expressing CD8+ T cells from healthy donors or patients, is shown to enhance T cell proliferation and cytokine secretion in the examples, suggesting autocrine production could enhance the functionality of a CXCR3⁺ modified T cell. The biological function of a CXCL9, CXCL10 and CXCL11 transgene, could be assessed by whether it provokes the same increase in cytokine production compared to the provided CXCR3 ligand transgene sequences, using an assay, or CFSE dilution of antigen stimulated CXCR3+ cells as in FIG. 4.

A second aspect of the invention provides an isolated preparation of immune cells, particularly a preparation of T cells, wherein the isolated preparation of immune cells comprises at least (≥) 50%, particularly 70%, more particularly ≥80%, even more particularly ≥90% immune cells, particularly T cells, which express a human CXCR3 variant selected from CXCR3A, CXCR3alt+, and/or CXCR3B, particularly when the human CXCR3 variant, or one of the human CXCR3 variants is CXCR3A and/or CXCR3alt. In some embodiments, the population of T cells expressing CXCR3 variants is a directly ex vivo sample, in others, the isolated population has undergone an enrichment, expansion, or transgene insertion procedure to increase the CXCR3+ population from a starting sample of immune cell precursors.

In certain embodiments, a majority of the preparation of immune cells express only CXCR3alt, only CXCR3B, or only CXCR3A.

In certain embodiments, a majority of the preparation of immune cells express CXCR3A and CXCR3B, or CXCR3alt and CXCR3B. In particular embodiments the majority of immune cells in the preparation of immune cells express CXCR3A and CXCR3alt together.

In certain embodiments, a majority of the preparation of immune cells express all three CXCR3alt, CXCR3A and CXCR3B proteins.

This aspect of the invention encompasses both native CXCR3 variant expression by the preparation of immune cells, or CXCR3 variant expression from a transgene. The CXCR3 expression may be confirmed by realtime qPCR, for example using the probes provided herein, wherein a signal >0.1 fold, particularly >0.2 fold the expression of the house-keeping genes is considered positive. In order to confer the desired functional phenotype, it is understood that these CXCR3 variants should be expressed as a protein present at the cell surface. The cells may be isolated from peripheral blood, or peripheral blood mononuclear cells, a tissue sample, and/or a tumour sample, and may comprise additional transgenic proteins. In certain embodiments of the invention with relevance for cancer immunotherapy, the isolated preparation of immune cells according to the above aspect of the invention, has been isolated from, or expanded from, a plurality of immune cells isolated from a cancer patient sample, such as peripheral blood, or tumour infiltrating lymphocytes (TIL), and/or derived from lymph nodes draining tumour tissue (Poch et al. Oncoimmunol. 2018, 7(9):e1476816); Dudley et al. J. Immunother. 2003, 26: 332; Sakellariou et al. 2018, 36:95).

The data presented in the examples demonstrates the ability of CXCR3⁺ T cells derived from blood to respond to CXCR3 ligand migration signals (FIGS. 3 and 4), and to produce inflammatory cytokines IFNγ and TNFα after stimulation with autologous tumour lysate or viral antigen by (FIG. 2, FIG. 4). These cells may serve as a starting point for an enriched preparation of native antigen specific, CXCR3+ cells, as they are early effector cells associated a stronger immune response, and better clinical outcomes in cancer. Alternatively, they may be a useful starting population for introducing additional transgenes such as CXCR3 variants, CXCR3 ligands, TgTCR, CAR T cell receptors in transgenic approaches.

In another embodiment, the invention provides an isolated population of immune cells comprising 50%, particularly ≥70%, more particularly ≥80% CXCR3⁺ genetically modified immune cells according to any embodiment of the first aspect of the invention. In other words, primary cells which express CXCR3A, CXCR3B, and/or CXCR3alt from a transgene, and optionally an artificial antigen receptor, and/or a CXCR3-binding chemokine.

In certain embodiments, the isolated preparation of CXCR3+ cells according to this aspect of the invention is ≥50%, particularly ≥70%, more particularly ≥80% enriched in one of the following functionally distinct T cell subsets:
a. CD8⁺ T cells, particularly CD8⁺CCR7⁺CD45RA⁺CD95⁺ stem-like memory T cells, and/or CD8⁺CCR7⁺CD45RA⁻ central memory T cells,
b. CD4⁺ memory T cells, particularly T helper type 1, T-bet⁺CD4⁺ memory T cells,
c. CD4⁺ Treg cells, particularly CD4⁺CD25⁺ Treg cells, expressing foxp3, or
d. NK or NKT cells, particularly CD56+NK or NKT cells.

The data presented in the examples demonstrates that CXCR3 variants are highly expressed on CD8⁺CCR7⁺CD45RA⁺CD95⁺ stem-like memory T cells, and CD8⁺CCR7⁺CD45RA⁻ central memory T cells in healthy blood and BC patient LN. These native CXCR3⁺ cells may be purified to provide an isolated preparation of CXCR3⁺ cells according to the invention. In embodiments where CD4 help, cytotoxic NKT cell activity, or suppressive Treg cytokines are desirable in the isolated preparation of cells, the alternative embodiments b. through c. may be used. Th1 and NKT cells are useful for fighting cancer, bacterial or viral infections, whereas Tregs may suppress inflammation caused by, for example autoimmune or graft-specific immune activation. The expression of subset-identifying surface molecules can be determined for example, by flow cytometry in comparison to an isotype control, or a cell that is known to lack the marker in question, or by realtime qPCR.

In another embodiment, the modified T cells, or the isolated preparation of CXCR3+ cells according to the first or second aspect of the invention respectively, are defined by their performance in at least one assay which confirms the functionality of the transgenic CXCR3 variant expression. The concentration of CXCL9, CXCL10, or CXCL11 used in any of the following assays may be in the range of 1 to 1000 ng/ml of CXCL11, particularly between 10 and 100 ng/ml. A titration of the concentrations 1, 10, 100 and 1000 ng/ml is of particular use, with an unstimulated control to identify a kinetic, or functional response to stimulation.

According to this embodiment, the cell, or the population of cells may have a chemotactic index>2 upon in vitro stimulation with CXCL9, CXCL10, and/or CXCL11, using an assay such as that provided on p.23, I.30 of the examples.

Alternatively, the cell or population of cells may have a higher frequency of proliferating cells upon stimulation with antigen and CXCL11, compared to non-stimulated cells, in other words the ratio of the measurement of proliferation of the isolated CXCR3+ population, compared to a pre-isolation, or un-isolated sample, must be >1. This may be measured using an in vitro cell division assay such as flow cytometry measurement of the percent of cells which dilute a labelling dye in an assay such as that provided in the methods on p.24 I.23 (alternative dyes, see Parish Immunol. Cell Biol. 1999, 77(6):499), or, for example, by thymidine incorporation. Alternatively, or in addition to proliferation, an assay to measure enrichment of a CXCR3+ antigen specific population may be used, such as the assay measuring CD137 upregulation after antigen culture provided on p.24, I.6. For the current invention, the fold enrichment of the CXCR3+ antigen specific population in response to antigen and CXCL11 stimulation must be >1, particularly over 1.2 times greater than an unfractionated control sample.

In further options provided by this embodiment, in comparison to an unmodified T cell lacking a CXCR3 transgene or transgenes, or an un-fractionated, or pre-isolated preparation of cells, the T cell, or the isolated CXCR3+ population of cells is characterised by having enhanced lytic potential. Lytic potential may be measured by % killing of a target cell population, such as tumour, or virus infected cells marked with a dye or radiolabel, or intracellular measurement of the lytic enzymes granzyme B and/or perforin in a flow cytometric assay (Amini et al. Front. Immunol. 2019, 10:1148; Wagner et al. Nat. Med. 2019, 25(2):242). The ratio of % target cells killed of the CXCR3+ population of cells, must be >1, particularly >1.5 times that of an unfractionated, or pre-isolation sample.

Lastly, the CXCR3+ cell, or population of cells may produce more effector cytokines, particularly effector cytokines selected from IFNγ, TNFα, IL-10, and IL-2, following stimulation with CXCL11, and optionally, antigen-pulsed antigen presenting cells, a TCR crosslinking agent and soluble antigen, or a calcium flux inducing agent. An exemplary protocol of 12 hours stimulation with antigen expressing cells and CXCL11, in the presence of Brefeldin A, followed by intracellular flow cytometry of IFNγ and TNFα, is provided on p.24, I.20. For the purposes of the invention, the % of cytokine+ cells in the expanded, or enriched CXCR3+ population of cells should by >1 times, particularly >1.1 times greater than that of an unenriched, or pre-enrichment control sample.

The data presented in the examples demonstrates that in cancer patients, inflamed lymph nodes rich in CXCR3 ligands are more highly infiltrated with CXCR3⁺ T cells, and tumours with more CXCR3 biomarker expression, particularly CXCR3A and CXCR3alt, show evidence of containing more T cells. Furthermore, in vitro assays demonstrate that CXCR3+ stem cell memory or central memory cells have a chemotactic index over 2 when stimulated to CXCL9, CXCL10, or CXCL11, and proliferate and produce more cytokines than other subsets in response to viral or tumour antigens when stimulated with CXCL11. These assays form the basis of the functional characterisation presented in this embodiment of the invention.

A third aspect of the invention provides a modified CXCR3⁺ T cell, or an isolated CXCR3⁺ preparation of cells, for use as a medicament. In some embodiments, this medicament is used to enhance an aspect of T cell immunity, encompassing both pro- and anti-inflammatory T cell functions provided by the different transgene-bearing sub-populations provided above.

In certain particular embodiments, the modified CXCR3⁺ T cell, or the isolated CXCR3⁺ preparation of cells is used to enhance CD8⁺ T cell immunity, particularly to improve immunity against a solid tumour selected from a squamous cell cancer or adenocarcinoma, more particularly a cancer selected from breast, colorectal, neuroblastoma, sarcoma, bladder, glioblastoma, hepatocellular, pancreatic, renal, gastrointestinal, or prostate cancer. The data in the examples suggests these cells are of particular use to treat a solid cancer, rather than a systemic form cancer derived from a lymphoid cell, as the CXCR3 transgene or enriched CXCR3 expression confers the ability to home to an inflamed tumour tissue expressing CXCL11, CXCL9, or CXCL10.

In an alternative embodiment of the CXCR3⁺ T cell, or the isolated CXCR3⁺ preparation of cells for use as a medicament, the cell, or cells are used to treat an infectious disease. As CXCR3 is shown to enhance inflammatory capability of CD8+ T cells, this treatment may be of particular use in treating disease caused by intracellular pathogens such as a chronic viral infection, where infected cells are vulnerable to killing by cytotoxic CD8 T cells. Relevant viral diseases include, but are not limited to EBV, CMV, human immunodeficiency virus, coronavirus, or hepatitis. Data presented in the examples shows that in vitro stimulation of CXCR3 variant expressing cells by CXCL11 is able to activate CMV and EBV specific CD8 T cells in a human sample (FIG. 4).

A fourth aspect of the invention provides a method of obtaining an isolated CXCR3⁺ preparation of cells, comprising a first step of providing a human sample comprising immune cells, particularly a peripheral blood sample, lymph nodes, or a tumour tissue sample. The data in the examples demonstrates that tumour-draining lymph nodes are enriched for CXCR3+, tumour specific cells. The tumour tissue of neoadjuvant-responsive BC patients was also shown to be enriched in the CXCR3 variant biomarkers.

Likewise, any target tissue, or the lymph nodes draining a target tissue, such as an organ inflicted by harmful immune infiltration, or viral infection, may serve as an appropriate starting sample in which either the desired antigen-specificity of CXCR3 expression profile is present.

The next step is to select, or enrich from the sample the immune cells, particularly T cells, which express the CXCR3 variants CXCR3alt+, CXCR3B and/or CXCR3A+, and/or to remove the cells which do not express the CXCR3 variants CXCR3A, CXCR3B and/or CXCR3alt. This may be achieved, for example, by magnetic sorting with CXCR3alt+ specific antibody bearing a magnetic bead and subsequent retention in a magnetic field, or by flow cytometric sorting of fluorescently labelled cells. The expression of total CXCR3, or individual variant combinations are encompassed by this method.

In some embodiments, the method to obtain an isolated CXCR3+ preparation of cells includes an additional gene transfer step, where a transgene is inserted into the plurality of cells. The transgene may comprise a CXCR3 variant nucleic acid sequence selected from SEQ ID NO 001, SEQ ID NO 002, SEQ ID NO 003, SEQ ID NO 004, SEQ ID NO 005, SEQ ID NO 006, and optionally a CXCR3 ligand sequence selected from SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015. Alternatively, the transgene nucleic acid sequence may encode an amino acid sequence that has ≥95% sequence identity, particularly wherein the transgene encodes an amino acid sequence that has ≥96%, ≥97, ≥98 or even ≥99% sequence identity to the amino acid sequence encoded by a sequence selected from SEQ ID NO 001, SEQ ID NO 002, SEQ ID NO 003, SEQ ID NO 004, SEQ ID NO 005, SEQ ID NO 006, SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015, respectively. In this case, the protein encoded by the sequence has essentially the same biological activity as one of the sequences provided above, particularly in assays such as T cell migration, cytokine production, or proliferation as outlined above and in the examples.

It may also be desirable to add a transgene for a TgTCR or CAR at this step as specified in the first aspect of the invention to provide a modified T cell.

A final embodiment of the method for preparing an isolated preparation of CXCR3 variant expressing cells, provides an expansion step, wherein the cells are cultured with CXCL9, CXCL10, and/or CXCL11 at a concentration between 1 to 1000, or particularly with 10-100 ng/ml of CXCL11. This may optionally be in the presence of antigen, and/or a gamma-chain cytokine, particularly IL-2.

The data in the examples, particularly the CXCR3 ligand titration migration assay shows that 10-100 ng/ml of CXCL9, CXCL10, and/or CXCL11 can enhance the proliferation, migration, and cytokine production of T cell subsets which express CXCR3. The functional importance of the in vitro assays is confirmed in the biomarker analysis capturing the direct correlation between the presence of CXCR3 chemokine family molecules in BC tumours, and positive outcomes of neoadjuvant cancer treatment, and overall survival.

The invention further relates to the following items;
A. A modified CD3⁺ T cell expressing a CXCR3 transgene, wherein the transgene encodes a recombinant protein comprising a human CXCR3 variant selected from:
   CXCR3A, CXCR3alt+, and/or CXCR3B,
   particularly wherein the human CXCR3 variant, or one of the human CXCR3 variants is CXCR3A and/or CXCR3alt.
B. A modified T cell according to item A, wherein the T cell is a CD3+ CD8+ memory T cell.
C. The modified T cell according to item A or B, wherein the CXCR3 transgene
   a. comprises the reverse complement of the premRNA transcript of CXCR3A, CXCR3alt, and/or CXCR3B, particularly a sequence selected from SEQ ID NO 001, SEQ ID NO 002 and/or SEQ ID NO 003, or
   b. comprises the reverse complement of the coding mRNA transcript of CXCR3A, CXCR3alt, and/or CXCR3B, particularly a sequence selected from SEQ ID NO 004, SEQ ID NO 005, and/or SEQ ID NO 006, or
   c. encodes an amino acid sequence that has at least (≥) 95% sequence identity to the amino acid sequence encoded by SEQ ID NO 001, SEQ ID NO 002, SEQ ID NO 003, SEQ ID NO 004, SEQ ID NO 005 and/or SEQ ID NO 006, and wherein the encoded protein has the same biological activity as SEQ ID NO 001, SEQ ID NO 002, SEQ ID NO 003, SEQ ID NO 004, SEQ ID NO 005 and/or SEQ ID NO 006,
particularly wherein the CXCR3 transgene encodes an amino acid sequence that has ≥96%, ≥97, ≥98 or even ≥99% sequence identity to the amino acid sequence encoded by SEQ ID NO 001, SEQ ID NO 002, SEQ ID NO 003, SEQ ID NO 004, SEQ ID NO 005 and/or SEQ ID NO 006.
D. The modified T cell according to any one of items A to C, wherein the expression level of CXCR3A and/or CXCR3alt is higher than the expression level of CXCR3B, particularly wherein the ratio of the expression level of CXCR3A and/or CXCR3alt in comparison to CXCR3B is more than 1.
E. The modified T cell according to any one of the items A to D, further expressing a chimeric antigen receptor (CAR) comprising
   a. a signal peptide,
   b. a target specific recognition domain, particularly wherein the target is selected from a tumour-associated surface antigen, a lineage-specific antigen, a tissue-specific surface antigen, or a virus-specific surface antigen,
   c. an effector domain comprising a transmembrane region and one or more intracellular signalling,
   d. a linker region, connecting domain (b) and domain (c).
F. The modified T cell according to any one of the items A to D, further expressing a transgenic T cell receptor (TgTCR) protein, wherein the TgTCR recognises a target selected from a tumour-associated surface antigen, a lineage-specific antigen, a tissue-specific surface antigen, or a virus-specific surface antigen.
G. The modified T cell according to items E or F, wherein the target specific recognition domain, or the TgTCR recognises a target selected from a transgenic T cell receptor specific for an antigen selected from LMPA, CMVₚₚ₆₅, GD2, L1CAM, Her2, IL13Ra2, EGFRvIII, CD133, mesothelin, CAIX, CEACAM5, TAG-72, CEA, COA-1, PSMA, or c-MET.
H. The modified T cell according to any one of the items A to G, wherein the cell further expresses a CXCR3 ligand transgene comprising a CXCR3 ligand transgene promotor sequence and a recombinant human CXCR3 ligand, and wherein the transgene comprises:
   a. the reverse complement of a premRNA transcript of CXCL9, CXCL10, and/or CXCL11, particularly a sequence selected from SEQ ID NO 007, SEQ ID NO 008, SEQ ID NO 009, SEQ ID NO 010 and/or SEQ ID NO 011, or
b. the reverse complement of a coding mRNA transcript of CXCL9, CXCL10, and/or CXCL11, particularly a sequence selected from SEQ ID NO 012, SEQ ID NO 013, SEQ ID NO 014 and/or SEQ ID NO 015, or
   c. a nucleic acid sequence encoding an amino acid sequence that has at least (≥) 95% sequence identity to the amino acid sequence encoded by SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015, and wherein the encoded protein has the same biological activity as the amino acid sequence encoded by SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015,
particularly wherein the CXCR3 transgene encodes an amino acid sequence that has ≥96%, ≥97, ≥98 or even ≥99% sequence identity to the amino acid sequence encoded by SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015.
I. An isolated preparation of immune cells, particularly a preparation of T cells, wherein the isolated preparation of immune cells comprises at least (≥) 50%, particularly 70%, more particularly ≥80%, even more particularly 90% immune cells, particularly T cells, expressing a human CXCR3 variant selected from CXCR3A, CXCR3alt+, and/or CXCR3B, particularly wherein the human CXCR3 variant, or one of the human CXCR3 variants is CXCR3A and/or CXCR3alt.
J. The isolated preparation of cells according to item I, wherein the cells are derived from a cancer patient sample, particularly a cancer patient sample selected from peripheral blood, tumour tissue and/or tumour draining lymph node tissue.
K. The isolated preparation of cells according to items I or J, comprising at least (≥) 50%, particularly ≥70%, more particularly ≥80% of any one of the modified immune cells as specified in items A to H.
L. The isolated preparation of cells according to item I or J, wherein the cells do not express any transgenes.
M. The isolated preparation of cells according to any one of the items I to L, wherein within the immune cells expressing a CXCR3 variant, ≥50%, particularly ≥70%, more particularly ≥80% are:
   a. CD8⁺ memory cells, particularly CD8⁺CCR7⁺CD45RA⁺CD95⁺ and/or CD8⁺CCR7⁺CD45RA⁻ CD95⁺ memory T cells
   b. CD4⁺ memory T cells, particularly T helper type I, T-ber CD4⁺ memory T cells,
   c. CD4⁺ T regulatory (Treg) cells, particularly CD4⁺CD25⁺ Treg cells, or
   d. NK or NKT cells, particularly CD56⁺ NK or NKT cells.
N. The modified T cell according to any one of the items A to H, or the isolated preparation of cells according to any one of items I to M, wherein the cell, or the population of cells is characterised by:
   a. a chemotactic index>2 upon in vitro stimulation with CXCL9, CXCL10, and/or CXCL11,
   b. a higher frequency of proliferating cells upon stimulation with antigen and CXCL11, compared to non-stimulated cells,
and/or wherein, in comparison to an unmodified T cell lacking a CXCR3 transgene, or transgenes, as specified item C, or an un-isolated preparation of cells, the T cell, or the isolated population of cells is characterised by:
   c. enhanced lytic potential, and/or
   d. producing more effector cytokines upon stimulation with CXCL11, particularly effector cytokines selected from IFNγ, TNFα, IL-10, and/or IL-2.
O. The modified immune cell according to any one of the items A to H, or the isolated preparation of cells according to any one of the items I to N, for use as a medicament.
P. The modified T cell according to any one of the items A to H, or the isolated preparation of cells according to any one of the items I to O, for use to enhance T cell immunity, particularly CD8⁺ T cell immunity.
Q. The modified immune cell according to any one of the items A to H, or the isolated preparation of cells according to any one of items I to P, for use in treating cancer, particularly a solid cancer such as a squamous cell cancer or adenocarcinoma, more particularly a cancer selected from breast, colorectal, neuroblastoma, sarcoma, bladder, glioblastoma, hepatocellular, pancreatic, renal, gastrointestinal, or prostate cancer.
R. The modified immune cell according to any one of the items A to H, or the isolated preparation of cells according to any one of the items I to P, for use in treating infectious disease.
S. A method of obtaining an isolated preparation of cells according to any one of the items I to N, comprising the steps of
   providing a sample comprising immune cells;
   selecting immune cells, particularly T cells, which express the CXCR3 variants CXCR3alt+, CXCR3B and/or CXCR3A+ present in the sample, and/or removing from the sample the cells which do not express the CXCR3 variants CXCR3A, CXCR3B and/or CXCR3alt.
T. A method of obtaining an isolated preparation of cells comprising the steps of
   providing a sample comprising a plurality of immune cells;
   in a gene transfer step, inserting a transgene into each of the plurality of immune cells, wherein the transgene comprises
      a. a nucleic acid sequence selected from SEQ ID NO 001, SEQ ID NO 002, SEQ ID NO 003, SEQ ID NO 004, SEQ ID NO 005, SEQ ID NO 006, and optionally SEQ ID NO 007, SEQ ID NO 008, SEQ ID NO 009, SEQ ID NO 010, SEQ ID NO 011, SEQ ID NO 012, SEQ ID NO 013, SEQ ID NO 014 and/or SEQ ID NO 015, or
      b. a nucleic acid sequence encoding an amino acid sequence that has at least (≥) 95% sequence identity to the amino acid encoded by a sequence selected from SEQ ID NO 001, SEQ ID NO 002, SEQ ID NO 003, SEQ ID NO 004, SEQ ID NO 005, SEQ ID NO 006, and optionally SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015, and wherein the encoded amino acid sequence has the same biological activity as SEQ ID NO 001, SEQ ID NO 002, SEQ ID NO 003, SEQ ID NO 004, SEQ ID NO 005, SEQ ID NO 006, SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015,
         particularly wherein the transgene encodes an amino acid sequence that has ≥96%, ≥97, ≥98 or even ≥99% sequence identity to an amino acid sequence selected from SEQ ID NO 001, SEQ ID NO 002, SEQ ID NO 003, SEQ ID NO 004, SEQ ID NO 005, SEQ ID NO 006, and optionally SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015;
      in an optional second gene transfer step, inserting a transgene encoding a CAR, or a TgTCR protein as specified in any one of the items E to G.
U. A method of preparing an isolated preparation of cells according to the items S or T, further comprising an optional expansion step, wherein the cells are cultured with CXCL9, CXCL10, and/or CXCL11 at a concentration between 1 to 1000 ng/ml, particularly with 10-100 ng/ml of CXCL11, optionally in the presence of antigen, and/or a gamma-chain cytokine, particularly IL-2.
A1. A modified CD3⁺ T cell, particularly a CD3+ CD8+ memory T cell, expressing a CXCR3 transgene, wherein the transgene encodes a recombinant protein comprising a human CXCR3 variant selected from:
   CXCR3A, CXCR3alt+, and/or CXCR3B,
   particularly wherein the human CXCR3 variant, or one of the human CXCR3 variants is CXCR3A and/or CXCR3alt.
A2. The modified T cell according to item A1, wherein the CXCR3 transgene
   a. comprises the reverse complement of the premRNA transcript of CXCR3A, CXCR3alt, and/or CXCR3B, particularly a sequence selected from SEQ ID NO 001, SEQ ID NO 002 and/or SEQ ID NO 003, or
   b. comprises the reverse complement of the coding mRNA transcript of CXCR3A, CXCR3alt, and/or CXCR3B, particularly a sequence selected from SEQ ID NO 004, SEQ ID NO 005, and/or SEQ ID NO 006, or
   c. encodes an amino acid sequence that has at least (≥) 95% sequence identity to the amino acid sequence encoded by SEQ ID NO 001, SEQ ID NO 002, SEQ ID NO 003, SEQ ID NO 004, SEQ ID NO 005 and/or SEQ ID NO 006, and wherein the encoded protein has the same biological activity as SEQ ID NO 001, SEQ ID NO 002, SEQ ID NO 003, SEQ ID NO 004, SEQ ID NO 005 and/or SEQ ID NO 006,
      particularly wherein the CXCR3 transgene encodes an amino acid sequence that has ≥96%, ≥97, ≥98 or even ≥99% sequence identity to the amino acid sequence encoded by SEQ ID NO 001, SEQ ID NO 002, SEQ ID NO 003, SEQ ID NO 004, SEQ ID NO 005 and/or SEQ ID NO 006.
A3. The modified T cell according to items A1 or A2, wherein the expression level of CXCR3A and/or CXCR3alt is higher than the expression level of CXCR3B, particularly wherein the ratio of the expression level of CXCR3A and/or CXCR3alt in comparison to CXCR3B is >1.
A4. The modified T cell according to any one of the items A1 to A3, further expressing a chimeric antigen receptor (CAR) comprising
   a. a signal peptide,
   b. a target specific recognition domain, particularly wherein the target is a tumour-associated surface antigen, a lineage-specific antigen, a tissue-specific surface antigen, or a virus-specific surface antigen,
   c. an effector domain comprising a transmembrane region and one or more intracellular signalling,
   d. a linker region, connecting domain (b) and domain (c).
A5. The modified T cell according to any one of the items A1 to A 3, further expressing a transgenic T cell receptor (TgTCR) protein, wherein the TgTCR recognises a target selected from a tumour-associated surface antigen, a lineage-specific antigen, a tissue-specific surface antigen, or a virus-specific surface antigen.
A6. The modified T cell according to any one of the items A4 or A5, wherein the target specific recognition domain, or the TgTCR recognises a target selected from a transgenic T cell receptor specific for an antigen selected from LMPA, CMVₚₚ₆₅, GD2, L1 CAM, Her2, IL13Ra2, EGFRvIII, CD133, mesothelin, CAIX, CEACAM5, TAG-72, CEA, COA-1, PSMA, or c-MET.
A7. The modified T cell according to any one of the items A1 to A6, wherein the cell further expresses a CXCR3 ligand transgene comprising a CXCR3 ligand transgene promotor sequence and a recombinant human CXCR3 ligand, and wherein the transgene comprises:
   a. the reverse complement of a premRNA transcript of CXCL9, CXCL10, and/or CXCL11, particularly a sequence selected from SEQ ID NO 007, SEQ ID NO 008 SEQ ID NO 009, SEQ ID NO 010 and/or SEQ ID NO 011, or
   b. the reverse complement of a coding mRNA transcript of CXCL9, CXCL10, and/or CXCL11, particularly a sequence selected from SEQ ID NO 012, SEQ ID NO 013, SEQ ID NO 014 and/or SEQ ID NO 015, or
   c. a nucleic acid sequence encoding an amino acid sequence that has at least (≥) 95% sequence identity to the amino acid sequence encoded by SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015, and wherein the encoded protein has the same biological activity as SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015,
      particularly wherein the CXCR3 transgene encodes an amino acid sequence that has ≥96%, ≥97, ≥98 or even ≥99% sequence identity to the amino acid sequence encoded by SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015.
A8. An isolated preparation of immune cells, particularly a preparation of T cells,
   wherein the isolated preparation of immune cells comprises at least (≥) 50%, particularly 70%, more particularly ≥80%, even more particularly ≥90% immune cells, particularly T cells, expressing a human CXCR3 variant selected from CXCR3A, CXCR3alt+, and/or CXCR3B, particularly wherein the human CXCR3 variant, or one of the human CXCR3 variants is CXCR3A and/or CXCR3alt.
A9. The isolated preparation of cells according to item A8, wherein the cells are derived from a cancer patient sample, particularly a cancer patient sample selected from peripheral blood, tumour tissue and/or tumour draining lymph node tissue.
A10. The isolated preparation of cells according to any one of the items A8 to A9, comprising at least (≥) 50%, particularly ≥70%, more particularly ≥80% of any one of the modified immune cells as specified in items A1 to A7.
A11. The isolated preparation of cells according to any of the items A8 to A10, wherein the cells do not express any transgenes.
A12. The isolated preparation of cells according to any one of the items A8 to A11, wherein within the immune cells expressing a CXCR3 variant, ≥50%, particularly ≥70%, more particularly ≥80% are:
   a. CD8⁺ memory cells, particularly CD8⁺CCR7⁺CD45RA⁺CD95⁺ and/or CD8⁺CCR7⁺CD45RA⁻ CD95⁺ memory T cells
   b. CD4⁺ memory T cells, particularly T helper type I, T-ber CD4⁺ memory T cells,
   c. CD4⁺ T regulatory (Treg) cells, particularly CD4⁺CD25⁺ Treg cells, or
   d. NK or NKT cells, particularly CD56⁺ NK or NKT cells.
A13. The modified immune cell according to any one of the items A1 to A7, or the isolated preparation of cells according to any one of the items A8 to A12, for use as a medicament.
A14. The modified immune cell according to any one of the items A1 to A8, or the isolated preparation of cells according to any one of the items A9 to A16, for use in
   a. treating cancer, particularly a solid cancer such as a squamous cell cancer or adenocarcinoma, more particularly a cancer selected from breast, colorectal, neuroblastoma, sarcoma, bladder, glioblastoma, hepatocellular, pancreatic, renal, gastrointestinal, or prostate cancer.
   b. treating infectious disease.
A15. A method of obtaining an isolated preparation of cells comprising the steps of
   providing a sample comprising a plurality of immune cells;
   in a gene transfer step, inserting a transgene into each of the plurality of immune cells, wherein the transgene comprises
      a. a nucleic acid sequence selected from SEQ ID NO 001, SEQ ID NO 002, SEQ ID NO 003, SEQ ID NO 004, SEQ ID NO 005, SEQ ID NO 006, and optionally SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015, or
      b. a nucleic acid sequence encoding an amino acid sequence that has at least (≥) 95% sequence identity to the amino acid sequence selected from SEQ ID NO 001, SEQ ID NO 002, SEQ ID NO 003, SEQ ID NO 004, SEQ ID NO 005, SEQ ID NO 006, and optionally SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015, and wherein the encoded amino acid sequence has the same biological activity as SEQ ID NO 001, SEQ ID NO 002, SEQ ID NO 003, SEQ ID NO 004, SEQ ID NO 005, SEQ ID NO 006, SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015,
         particularly wherein the transgene encodes an amino acid sequence that has ≥96%, ≥97, ≥98 or even ≥99% sequence identity to an amino acid sequence selected from SEQ ID NO 001, SEQ ID NO 002, SEQ ID NO 003, SEQ ID NO 004, SEQ ID NO 005, SEQ ID NO 006, and optionally SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015.
      in an optional second gene transfer step, inserting a transgene encoding a CAR, or a TgTCR protein as specified in any one of the items A5 to A7.

### Chemokine Biomarkers for Prediction and Stratification of Cancer Treatment

The present invention further relates to a method of a priori assessment of CXCR3 splice variants and its ligands CXCL9, CXCL10, and CXCL11 in muscle-invasive bladder cancer (MIBC) patients, to enable patients to be stratified for their predicted response to a chemotherapy drug treatment, or clinical outcome. The invention further relates to the treatment of cancer patients having been identified as being susceptible to certain treatment regimes.

### Summary of Diagnostic Aspects of the Invention

A next aspect of the invention relates to a method to measure T cell activity state in a patient tissue sample, wherein the method comprises the following steps; Firstly, providing a patient tissue sample, then in a measurement step, determining a biomarker expression level of the biomarkers CXCL11, and at least one of CXCR3A or CXCR3alt. The biomarker expression level of CXCR3B, CXCL4, CXCL9 and CXCL10 may also optionally be determined. In some embodiments, the tissue sample is assigned a classification which reflects the presence of the CXCR3 cytokine system an T cell activity state in the patient tissue sample, based on the biomarker expression levels.

Another aspect of the invention relates to a method to measure T cell activity state specifically in a patient tumour sample, wherein the method comprises the steps of providing a cancer tissue sample; then in a measurement step, determining a biomarker expression level of the biomarkers as specified above. In an optional classification step, the cancer tissue sample is assigned a value which reflects the number and activation status of the T cells present in the cancer tissue sample, based on the biomarker expression levels.

A next aspect provided by the invention is determining the expression level of the biomarkers CXCL11, and at least one of CXCR3A or CXCR3alt in a tumour sample taken from a patient who has been previously diagnosed with cancer, in order to predict the outcome of an antineoplastic treatment, or to classify the patient as a treatment responder or non-responder.

These methods applied in cancer may optionally include further measurements of the CXCR3B splice variant, and/or the additional chemokine biomarkers CXCL4, CXCL9, and CXCL10. A particular embodiment regards the classification of cancer patients according to the invention into treatment responders and non-responders. This may be based on the input of the specified biomarkers into an algorithm which provides a probability that the patient will respond favourably to an antineoplastic treatment, comparing the patient biomarker expression levels to representative reference samples, or comparing the biomarker expression levels in sample to a list of biomarker expression thresholds generated from previously analysed cohorts of patient biomarker expression data.

In one embodiment the expression level of the biomarker CXCL11 is determined at the level of protein expression. In certain embodiments of the method according to the invention, this value is expressed as a non-transformed expression level, or the expression level value of CXCL11 undergoes a normalisation process, particularly to the mass of sample analysed, or Area sinus hyperbolicus (arsinh) normalisation to provide a value as the arsinh of the expression per gram of tissue. Patients, or samples with an expression level of CXCL11 above a threshold of 13.98, particularly more than 22.4 pg per 10 mg sample tissue may be classified as treatment responders according to the invention.

Another particular aspect of the invention is a method to measure the expression of mRNA of the CXCR3 isoforms CXCR3A, CXCR3alt and/or CXCR3B, particularly using a methodology making use of nucleic acid probes that can differentiate between the sequences of the biomarker CXCR3 variants. This method may optionally include the normalisation of the CXCR3 variant expression levels with respect to the expression of house-keeping genes, particularly the house keeping genes IP08 and CDKN1B.

In some aspects of the invention, CXCR3 splice variant expression above a threshold level is used to pair patient cancer samples with a predicted clinical outcome. A patient with a tumour sample classified as positive, or high for a splice variant, is likely to respond to an antineoplastic treatment when CXCR3A is more than 2^{(-12.3)} more than that of the HKG, particularly more than 2^{(-11.97)} times that of the HKG, and/or CXCR3alt is more than 2^{(-13.8)} more than that of the HKG, particularly more than 2^{(-11.27)} times that of the HKG, and/or CXCR3B is more than 2^{(-11.9)} more than that of the HKG, particularly more than 2^{(-8.43)} times that of the HKG. The method of the invention may be particularly useful in predicting the clinical outcome of an antineoplastic treatment in cancer patients diagnosed with kidney, prostate, breast, lung, ovarian, gastric, rectal, melanoma, oesophageal, or particularly bladder cancer, more particularly muscle invasive bladder cancer. A particularly useful embodiment of the invention is the measurement of CXCL11 and CXCR3 splice variant biomarkers to predict the outcome of a patient to a neoadjuvant chemotherapy treatment, particularly an antineoplastic drug, Bacillus Calmette-Guerin, or an immune checkpoint modulator.

Another aspect of the invention is a method to stratify cancer patients according to the priority in which they should receive an antineoplastic surgical intervention, according to the expression levels of the specified biomarkers.

An additional aspect of the invention provides a method to predict the clinical outcome of an immune cell transfer treatment, by matching the expression level of CXCR3 biomarkers in a cell transfer sample, with the expression level CXCR3 ligands in a target tissue sample.

A further aspect of the invention is a method to compare the expression levels of CXCL11 and at least one of CXCR3A and/or CXCR3alt, and optionally CXCR3B, CXCL4, CXCL9, and/or CXCL10, in both a pre-treatment and post-treatment patient tumour sample, for the purpose of monitoring tumour progression over time.

A further aspect of the invention is a pharmaceutical composition which includes antineoplastic platinum drug, such as cisplatin, for use in the treatment of a cancer patient who has been classified as a drug responder according to any of the methods specified above.

A final aspect of the invention is a system for assigning a value to a patient tissue sample that reflects the T cell activity state of the tissue, comprising determining the expression level in the tissue sample of at least one of the biomarkers CXCL11, CXCR3A, CXCR3alt, CXCR3B, CXCL4, CXCL9, and/or CXCL10.

### DETAILED Description of Diagnostic Aspects of the Invention

One aspect of this invention relates to a method to measure the T cell activity state in a patient tissue sample, which may be of use in a clinical setting where information about the number and phenotype of T cells responding to CXCR3 binding-chemokines may inform the prognosis, or clinical treatment options of a patient. This first aspect of the invention may have significance in patients diagnosed with, or who are suspected of having, a condition characterised by chronic inflammation. Biomarker expression may allow a physician to measure whether sufficient pre-existing T cells are present in a target tissue, in order to predict whether a drug or cell-based treatment that enhances T cell responses will be effective, for example to combat viral infection or cancer. Conversely, the methodology may be used to examine a tissue sample for the presence of harmful, autoimmune T cell recruitment and activation, in order to inform decisions regarding immune-suppressing medication.

The first step of the method to measure T cell activity state, is providing a patient tissue sample. This may be peripheral blood or white blood cells, or in any tissue sample in which it is desirable from a clinical perspective to estimate the number or activation status of T cells within the tissue, such as a biopsy, or tissue derived from a tumour, graft, transplant, or a tissue targeted by an infection or autoimmune inflammation.

The second step of the method is the measurement of the expression levels of the chemokine CXCL11 and either, or both of, the CXCR3 variants CXCR3A or CXCR3alt that are present in the tumour sample. The measurement step may optionally include the determination of the expression level of the additional biomarkers, CXCL4, CXCL9, CXCL10, and/or the splice variant CXCR3B. In an optional classification step, the biomarker expression levels are used to assign the sample, or patient, a value which reflects the T cell activity state, in other words, the number and/or inflammatory potential of the T cells present in the patient tissue sample.

The data in the examples shows the efficacy of the CXCR3 and CXCR3 ligand biomarker family in identifying tissue with a potent T cell population that can be activated by subsequent chemotherapy. Furthermore, the expression of CXCR3 receptors on certain CD8+ T cell subsets is shown to confer migration, proliferation, and cytokine secretion potential to cytomegalovirus (CMV) specific T cells, as well as tumour-specific T cells in vitro, confirming the broad applicability of this method to both cancer, chronic viral infection, and potentially harmful autoimmune or graft-specific T cell responses.

A next aspect of the invention relates to a method to measure T cell activation specifically in a patient tumour sample, wherein the method comprises the steps of providing a cancer tissue sample; then in a measurement step, determining a biomarker expression level of the biomarkers as specified in the first aspect of the invention. This may be a peripheral white blood cell sample, a tumour sample from a biopsy, or tissue removed in a surgical intervention, or a cancer patient sample such as peri-tumoural tissue, or a tumour-draining lymph node. In an optional classification step, the cancer tissue sample is assigned a value which reflects the T cell activity state of the T cells present in the cancer tissue sample, based on the biomarker expression levels. The patient classification provided by the methods described herein may form part of the diagnostic protocols identifying a solid tumour, by helping to predict the prognosis of a patient with, or without cancer treatment. In other words, the method of the invention may be useful to stratify patients into groups with different recommended treatment protocols, or distinct patient outcomes, and may be of use to clinicians when, for example, assigning patients to groups which will receive either drug treatment, surgery, or palliative care.

In the data presented in the examples examining biomarker expression in BC patients, combining information about the level of CXCL11 expression in the tumour sample, with information about the expression of either of the CXCR3 splice variants CXCR3A, or CXCR3alt, classified patients with 100% accuracy as to whether they will respond favourably to antineoplastic neoadjuvant chemotherapy treatment. Using a linear regression model, the area under the curve of a Receiver operator characteristic (ROC) curve generated from the expression levels of either CXCL11 and CXCR3A, CXCL11 and CXCR3alt, or CXCL11 and CXCR3A and CXCR3alt is 1, showing complete accuracy in the prediction of NAC response. Further including CXCR3B expression level increased the fit of these predictive models. Cox-proportional hazard analysis of a second cohort showed confirmed an association between survival time and CXCL11, and showed CXCL4, CXCL9, CXCL10 mRNA, but not bulk CXCR3 mRNA level was correlated with BC treatment outcome.

Another aspect of the invention provides a method to predict whether a patient bearing a solid tumour will respond to a cancer treatment, by measuring the expression of selected biomarkers in a tumour sample as specified above: CXCL11 and at least one of CXCR3A or CXCR3alt, with the optional addition of CXCR3B, CXCL4, CXCL9 and/or CXCL10. The method provided herein may provide information that can help a clinician to choose the most appropriate personalised treatment course for the cancer patient. In the classification step of the method to predict the clinical outcome of a cancer patient, the expression levels of the CXCR3 chemokine family biomarkers determined in the sample are used to classify the cancer patient as either likely to be an anticancer treatment responder, or a treatment non-responder. In other words, the biomarker analysis method can make a prediction regarding the outcome of a clinical treatment for cancer.

A further aspect of the invention provides a method to measure the T cell activity state of a tumour, or a method to predict patient outcome in cancer treatment, by determining the expression level in a sample of cancer tissue of at least one of the biomarkers selected from a list comprising CXCL4, CXCL9, CXCL10, CXCL11, CXCR3A, CXCR3alt and/or CXCR3B. In one embodiment, the expression level of at least two, particularly at least three of the provided biomarkers are determined. As all the biomarkers in the CXCR3 system are associated with BC patient NAC treatment outcome, as the number of biomarkers measured increases, increasing accuracy can be achieved by in the statistical model to predict patient outcome.

From an analysis of mRNA (CXCR3) and protein (CXCL11) expression in a first MBIC patient cohort, the following combinations were identified as having the potential to predict neoadjuvant chemotherapy outcome with both 100% sensitivity and 100% specificity:
CXCL11 together with CXCR3alt or
CXCL11 together with CXCR3A, or
CXCL11 together with any two CXCR3 variants, or
CXCL11 together with all three CXCR3 variants.

Combining the expression of all four molecules in a predictive linear regression model gave the best fit to the Swedish cohort data in the examples, as reflected by the low AIC and Brier scores for this combination of predictive biomarkers. In the second cohort of patients, CXCL11, but also CXCL9 and CXCL10 mRNA expression also correlated with the amount of the T cell marker CD3 found in the tumour. In this cohort all four CXCR3 ligands CXCL4, CXCL9, CXCL10 and CXCL11 were associated the outcome of chemotherapy in bladder cancer patients, providing the following additional biomarker combinations which the potential to predict neoadjuvant chemotherapy outcome:
CXCL4 together with CXCR3alt or CXCR3A, or
CXCL4 together with any two CXCR3 variants,
CXCL4 together with all three CXCR3 variants
CXCL9 together with CXCR3alt or CXCR3A,
CXCL9 together with any two CXCR3 variants,
CXCL9 together with all three CXCR3 variants
CXCL10 together with CXCR3alt or CXCR3A,
CXCL10 together with any two CXCR3 variants,
CXCL10 together with all three CXCR3 variants
CXCL9 and CXCL10 together with CXCR3alt or CXCR3A,
CXCL9 and CXCL10 together with any two CXCR3 variants,
CXCL9 and CXCL10 together with all three CXCR3 variants,
CXCL10 and CXCL11 together with CXCR3alt or CXCR3A,
CXCL10 and CXCL11 together with any two CXCR3 variants,
CXCL10 and CXCL11 together with all three CXCR3 variants,
CXCL9 and CXCL11 together with CXCR3alt or CXCR3A,
CXCL9 and CXCL11 together with any two CXCR3 variants,
CXCL9 and CXCL11 together with all three CXCR3 variants,
CXCL4 and CXCL9 together with CXCR3alt or CXCR3A,
CXCL4 and CXCL9 together with any two CXCR3 variants,
CXCL4 and CXCL9 together with all three CXCR3 variants,
CXCL4 and CXCL10 together with CXCR3alt or CXCR3A,
CXCL4 and CXCL10 together with any two CXCR3 variants,
CXCL4 and CXCL10 together with all three CXCR3 variants,
CXCL4 and CXCL11 together with CXCR3alt or CXCR3A,
CXCL4 and CXCL11 together with any two CXCR3 variants,
CXCL4 and CXCL11 together with all three CXCR3 variants,
CXCL9, CXCL10 and CXCL4 together with CXCR3alt or CXCR3A,
CXCL9, CXCL10 and CXCL4 together with any two CXCR3 variants,
CXCL9, CXCL10 and CXCL4 together with all three CXCR3 variants,
CXCL9, CXCL10 and CXCL11 together with CXCR3alt or CXCR3A,
CXCL9, CXCL10 and CXCL11 together with any two CXCR3 variants,
CXCL9, CXCL10 and CXCL11 together with all three CXCR3 variants,
CXCL9, CXCL4 and CXCL11 together with CXCR3alt or CXCR3A,
CXCL9, CXCL4 and CXCL11 together with any two CXCR3 variants,
CXCL9, CXCL4 and CXCL11 together with all three CXCR3 variants,
CXCL4, CXCL10 and CXCL11 together with CXCR3alt or CXCR3A,
CXCL4, CXCL10 and CXCL11 together with any two CXCR3 variants,
CXCL4, CXCL10 and CXCL11 together with all three CXCR3 variants,
CXCL4, CXCL9, CXCL10 and CXCL11 together with CXCR3alt or CXCR3A,
CXCL4, CXCL9, CXCL10 and CXCL11 together with two CXCR3 variants, or
CXCL4, CXCL9, CXCL10 and CXCL11 together with all three CXCR3 variants.

In one embodiment according to these aspects of the invention, the expression level of CXCR3alt and CXCL11 alone is sufficient to determine cancer patient outcome.

In certain embodiments of the method to predict the outcome of an antineoplastic treatment in a patient bearing a solid tumour, the classification step comprises inputting the biomarker expression levels into a model-fitting statistical methodology in order to generate a value reflecting the probability that a patient will be an antineoplastic treatment responder. Statistical machine learning techniques, particularly supervised machine learning techniques which may be particularly useful for this method include, but are not limited to, random forest methodology, or neural networks. The use of logistic regression based on the results of previously analysed cancer patients is a particularly useful method to capture the relationship between expression levels and clinical response to provide an algorithm which generates a probability of drug response upon the input of biomarker data from the tumour sample. The classification or regression algorithms used in this aspect of the invention may be applied in order to improve the predictive power of the biomarkers at population level. It is understood that these methods may take variables other than the chemokine biomarker expression levels into account, such as variables selected from, but not limited to, age, gender, comorbidities, or clinical parameters.

The CXCL11 and CXCR3 expression levels in the Swedish cohort shown in the examples were incorporated into a predictive logistic regression model to test the benefit of using one, two, or multiple biomarker expression level values to predict BC patient outcome. The performance of each model in terms of predicting outcome to MVAC therapy was assessed by the area under the ROC curves, and both the AIK and Brier model fitting scores increased as more biomarkers were included. This analysis identified equations with which the pre-treatment tumour expression levels of CXCL11, together with CXCR3A, and particularly CXCR3alt, most accurately segregate muscle-invasive bladder cancer patients into MVEC neoadjuvant treatment responders and non-responders.

One possible embodiment of the method of predicting the outcome of an antineoplastic treatment in a patient bearing a solid tumour, is the classification of the cancer patient as an antineoplastic treatment responder if the level of one, or several biomarkers is above a certain threshold. Conversely, the cancer patient can be classified as an antineoplastic treatment non-responder if the level of biomarker expression is below a particular threshold. Useful thresholds and confidence intervals for these cut-offs are provided in the examples. This information could help a clinician to stratify patients into those who should receive prompt neoadjuvant treatment, and those who will benefit from surgery or other treatment options.

A particularly useful embodiment of the method to predict the outcome of an antineoplastic treatment in a patient with a solid tumour comprises measuring the level of CXCL11 protein in the sample. The expression of a marker may be assayed at the protein level via techniques such as fluorescence microscopy, flow cytometry, ELISPOT, ELISA or multiplex analyses. Marker expression may also be evaluated by measuring the expression at the level of mRNA by means of quantitative realtime PCR (qPCR), microarray, or sequencing assays.

Methods that use an antibody, or antibody fragment, which binds specifically to CXCL9, CXCL10, or CXCL11, such as ELISA, or bioplex, are particularly useful for determining the expression level of CXCR3 ligand protein in a tumour. Optionally, CXCR3 isoform expression levels can also be measured at the protein level, using molecular probes that distinguish between the CXCR3 splice variants. It is understood that the accurate measurement of protein according to certain embodiments of the invention is most effective in samples that have been preserved in such a way that protein is not degraded. It is preferable that the sample, or a portion of the sample, should be immediately frozen in liquid nitrogen upon resection from the patient, and stored at −80 degrees Celsius before processing, preferably in the presence of protein inhibitors.

In an alternative embodiment of the method according to the invention, the cancer patient is classified as a treatment responder, or non-responder, by comparing the expression levels of the listed biomarkers determined in the tumour sample with the expression levels in previously-analysed reference samples, where the matched clinical outcome is already known. For example, if the biomarker expression is equal to, or more than the expression level in a positive reference sample comprising tumour tissue from a NAC responder, the patient may be classified as likely to respond to NAC. Conversely, if the biomarker expression in a patient sample is equal to, or less than a negative reference sample of tumour tissue derived from a NAC non-responder, the patient may be classified as unlikely to respond to NAC. For the inversely correlated markers CXCR3B and CXCL4, this relationship is reversed.

In certain embodiments of the method provided by the invention, the expression level of biomarkers measured at the protein level, for example, CXCL11, undergoes a statistical normalisation process, for example, is expressed as a concentration per milligram of sample protein. This can be particularly advantageous when multiple samples are analysed in bulk, in order to standardise variance between samples to achieve normal distribution, and make biomarker expression levels more amenable to further statistical manipulations. The biomarker expression value determined by a method such as ELISA, or mass spectrometry, can be transformed to stabilise the distribution to compensate for repeated sampling procedures. The normalisation may include inputting the expression level into a transformation or scaling function, selected from, but not limited to, biexponential, logicle, or log transformation functions, or particularly an arsinh normalisation function.

In the data provided in the examples, bladder cancer patients can be accurately classified as neoadjuvant treatment responders, if the expression level of CXCL11 in the tumour sample taken prior to treatment is above 13.98, particularly more than 22.4 pg per 10 mg sample tissue. This useful threshold value was generated from the current model cohort, and may be subject to further changes as the predictive model is refined by the addition of more patient data in future use or studies.

In particularly advantageous embodiments of the invention, the expression levels of CXCL11, CXCR3A, CXCR3alt and/or CXCR3B are measured at the mRNA level. These measurements can be made with nucleic acid probes, particularly with a quantitative PCR methodology such as real time PCR, sequencing reactions, or a nucleic acid array. It is understood that accurate measurement of CXCR3 splice variant expression according to certain embodiments of the invention is most effective in samples that have been preserved in such a way that mRNA is not degraded. For example, the sample, or a portion of the sample as in the examples, should be frozen in liquid nitrogen, and stored at −80 degrees Celsius. Processing of the samples should be carried out on ice, optionally in the presence of RNase inhibitors.

One methodology that is particularly useful for measuring the CXCR3 splice variant expression level, is a nucleic acid amplification method conducted using polymerase chain reaction of the RNA extracted from the patient tumour sample. Specific nucleic acid probes, such as the primers of sequences SEQ ID NO 016 to 021 presented in the examples, can distinguish between the three CXCR3 variants using primer designs which target differently spliced regions, using standard Taqman ABI assay conditions.

In the data provided in the examples, combining information derived from qPCR measurements of the CXCR3 splice variants CXCR3A, CXCR3alt and CXCR3B all improved the prediction of BC clinical outcomes, compared to the CXCL11 expression level alone. The expression levels of the CXCR3 splice variants may optionally be determined using other technologies designed to quantify nucleic acids, including, but limited to, sequencing, microarrays or gene chips, for example a cDNA array.

In several embodiments of the invention it is particularly advantageous to compare, or normalise the expression of the biomarkers to the expression of one, or several, housekeeping genes. Two such genes which are of particular use are the genes IP08 and CDKN1B, but the skilled artisan will recognise that other stably expressed genes may be substituted by genes selected from, but not limited to, GAPDH, ACTB, B2M, PPIA, HPRTI, PGKI, TBP or TFRC.

In the data presented in the examples, statistical analysis of a range of housekeeping genes identified IP08 and CDKN1B as those most stably expressed in bladder cancer tumour samples. The mean expression of IP08 and CDKN1B was used to normalise biomarker expression values across samples of differing size, mRNA quality, or amplification level.

In an alternative embodiment of the cancer patient classification step provided by the invention, the patient is assigned as an antineoplastic treatment responder if the expression level of
CXCR3A is more than 2^{(-12.3)} time the expression level of the HKG, particularly more than 2^{(-11.97)} times the expression level of the HKG, and/or
CXCR3alt is more than 2^{(-13.8)} times the expression level of the HKG, particularly more than 2^{(-11.27)} times the expression level of the HKG, and/or
CXCR3B is more than 2^{(-11.9)} times the expression level of the HKG, particularly more than 2^{(-8.43)} times the expression level of the HKG.

For the CXCR3 variant biomarker thresholds provided here, the relationship between the biomarker and the HKG is the same when thresholds are used independently, or separately. In other words, using the example of CXCR3A above, the threshold for a positive outcome for neoadjuvant treatment may be assigned to a sample if the expression level CXCR3A is more than 2^{(-11.97)}, which is approximately 0.00025 times less than the expression level of the HKG. Determining the expression level of more than one of the provided biomarkers will increase the accuracy of the patient outcome prediction provided in the classification step. If markers are combined in a multivariate predictive model, in some cases an inverse correlation between biomarker level and outcome may be used to classify samples. In aspects of the invention utilising multivariate classification techniques (e.g. Random Forest), multiple “thresholds” are sequentially applied, and can function in both positive and negative correlation to the HKG.

In certain embodiments of the invention, in addition to, or instead of comparison to HKG, the expression level of the biomarker is compared to a baseline, or reference sample. One example of a negative control, or negative reference is a sample of healthy tissue from the affected organ, in which T cell infiltration is low, or absent. An example of a positive control, or positive reference sample, may be an example of a previously analysed sample in which there is a high level or T cell infiltration in the tumour or tissue sample. The skilled artisan will appreciate that in addition to analytical controls such as the examples presented above, patient samples may be compared to a range of pre-determined calibration samples, or standards, to provide appropriate technical and biological controls.

The skilled artisan will appreciate that the fold change CXCR3 variant expression values provided above are examples of differential cycle thresholds compared to HKG, i.e. the number of qPCR cycles needed to generate a fluorescence signal from the specific nucleic acid probes used, above a user-defined threshold. These values therefore reflect the PCR conditions and cycle threshold used to generate proof of principal evidence for the predictive model, and the exact values are expected to vary in practice.

In certain embodiments of the method according to the invention, the cancer tissue sample in which the biomarker levels are determined comprises, or essentially consists of, neoplastic cells derived from a solid tumour, but may further include heterogenous cells derived from the immune system or the tissue of origin. The method for predicting the clinical outcome of a patient is particularly useful for analysing neoplastic cells derived from squamous cell cancer, such as melanoma, or adenocarcinoma, particularly neoplastic cells from tissues selected from, but not limited to, tumours frequently treated with a combination of neoadjuvant treatment and surgery selected from breast, lung, kidney, prostrate, ovarian, colorectal, gastric, oesophageal or bladder cancer. The methods are thought to be particularly effective when the neoplastic cells themselves are not characterized by significant expression of CXCR3. In this way, the signal from the CXCR3 measurements can be certain to reflect the phenotype of T cells present in the tumour. In a particular embodiment of the method provided by the invention, the cancer patient is a bladder cancer patient, particularly a patient having been diagnosed with muscle-invasive bladder cancer of urothelial, or squamous cell origin.

In the data presented in the examples, an equation which captures the relationship between the expression levels of the biomarker CXCL11, together with the expression level of one or more of the CXCR3 splice variants, classified muscle-invasive bladder cancer patients with perfect accuracy as either responders, or non-responders to a subsequent treatment with MVEC neoadjuvant chemotherapy. The data presented in the examples demonstrates that in a single cell sequencing analysis of comparing bladder cancer and melanoma patient biopsies, the same immune cells express CXCR3 and CXCL11. In addition, studies show tumour-infiltrating, stem cell memory T cells, which the data provided here show are responsive to CXCR3 ligands, promote survival in kidney, prostrate, bladder, lung cancer and melanoma (Jansen C. S. et al. Nature (2019) 576:465; Brummelman J. et al. J. Exp. Med. (2018) 215(10):2520; Siddiqui I. Immunity (2019) 50:195). The methods to predict clinical outcomes in cancer in which chemokine biomarkers are used to estimate the potency of T cell immunity provided here, are therefore likely to be broadly applicable in squamous cell cancers, such as melanoma, or adenocarcinomas, such as bladder cancer.

In particular embodiments, the antineoplastic treatment in question is a neoadjuvant antineoplastic drug, particularly a neoadjuvant antineoplastic drug selected from, but not limited to, cisplatin, methotrexate, vinblastine, doxorubicin, carboplatin, adriamycin, gemcitabine, paclitaxel, filgramastim, pemetrexed, vinorelbine, oxaliplatin, vinflunine, or doxetaxel. In other words, the method is of particular use for predicting the outcome of a classical regime of MVEC or MVAC neoadjuvant chemotherapy containing the drugs methotrexate, vinblastine, doxorubicin or epirubucin, and cisplatin, but is likely to apply to similar drugs.

In certain embodiments of the method to predict the clinical outcome of a patient with a solid tumour provided by the invention, the patient sample is tumour tissue or lymph node tissue, particularly a tumour tissue sample taken during a biopsy to perform pathology typing of the tumour, or a sample taken from a lymph node draining the vicinity of the tumour.

In another embodiment of the invention, the classification step of the method can be of use to stratify bladder cancer patients into groups with high or low priority for cystectomy. The method provided can be used to assign muscle invasive bladder cancer patients classified as antineoplastic chemotherapy treatment non-responders as specified above, into a group with high priority for receiving a radical cystectomy procedure, without prior neoadjuvant treatment. This classification can avoid disease progression or metastases during an ineffective treatment regime. Additional cancer types wherein this aspect of the invention may be usefully employed to aid clinical stratification of patients into groups which are likely to benefit from either neoadjuvant treatment or surgical intervention as a first line of treatment includes, but is not limited to, breast cancer, rectal cancer, oesophageal cancer and gastric cancer.

The data in the examples shows that bladder cancer patients classified as neoadjuvant treatment non-responders are unlikely to experience tumour down-grading upon chemotherapy treatment. Clinical outcomes in bladder cancer can be improved if these patients instead receive prompt surgical intervention.

Another aspect of the invention is a method to match the CXCR3 expression status of an immune cell transfer treatment product, with the CXCL4, CXCL9, CXCL10 and/or CXCL11 expression status of the patient tissue being targeted by the immune cell transfer. In other words, a method to match positive expression of the CXCR3 variants in the cells for transfer, with expression of their ligands in the target tissue, in order to ensure the treatment is likely to succeed. According to this aspect, the method can predict the outcome of an immune cell transfer cancer treatment, particularly a T cell transfer treatment in a patient bearing a solid tumour. Firstly, a patient or product provides a cell transfer sample, or an autologous immune cell sample, in addition to a target tissue sample, such as a patient tumour sample.

In a first cell transfer measurement step, the expression level of the biomarkers CXCR3A, CXCR3alt, and/or CXCR3B in the cell transfer sample is determined, and next, in a target tissue sample measurement step, the expression level of the biomarkers CXCL4, CXCL9, CXCL10, and/or CXCL11 is determined, according to the measurement protocols as specified in previous aspects of the invention. Lastly, in a classification step, the patient assigned a value, reflecting the likelihood, or probability that the cell transfer treatment will have a positive clinical outcome, if the biomarker expression levels are positive, or high, in both samples. This value reflects the potential of the cells to migrate to the target tissue, and their local potential for proliferation and cytokine production after transfer. This method may utilise algorithms, reference samples or thresholds to classify the patient sample for biomarker status, as provided in other methods of the invention provided above. This method may be particularly desirable for a personalised medicine strategy, wherein the chemokine sensitivity of a T cell transfer product, such as, for example, a T cell with a recombinant tumour-specific TCR, or a tumour-specific T cell population expanded from a donor, or patient's own tumour sample, is assessed for whether it will effectively respond to the chemokine ligands expressed by the patient's tumour prior to administration.

A next aspect of the invention, is the use of CXCR3 chemokine system biomarkers provided in a method to monitor tumour progression over time, or to monitor the presence of tumour immune infiltrate in a patient tumour over time. The method involves the step of providing two, or more than two, sequential tumour samples at different sampling times. In a measurement step, the expression level of CXCL11 and either, or both of CXCR3A and CXCR3alt are determined in the sequential tumour samples. Optionally, the expression level of CXCR3B, CXCL4, CXCL9 and/or CXCL10 can be measured. In a final classification step, the patient is classified as a treatment responder, or as a patient with increased tumour immunity, if the expression levels of the CXCL11, CXCR3A, CXCR3alt, CXCL9, or CXCL10 increase, and/or CXCL4 or CXCR3B decrease in later samples, compared to earlier samples. This embodiment of the invention is of particular use when comparing pre-treatment tumour samples, to post-treatment tumour samples, or providing information to a clinician on whether a patient is responding to a particular treatment regime over time.

The data presented in the examples demonstrates that increasing expression levels of the biomarkers CXCL11, CXCR3A, CXCR3alt, CXCL9, and CXCL10 correlates with improved survival following NAC-treatment, whereas CXCR3B and CXCL4 expression is lower in these patients.

Another aspect of the invention provides a neoadjuvant antineoplastic pharmaceutical formulation for use in the treatment of a patient with a solid tumour who has been classified as a drug responder according to the previously specified methods. Antineoplastic pharmaceutical formulations of particular utility according to the invention are those which comprise, or consist of an antineoplastic drug, particularly an antineoplastic drug selected from, but not limited to, cisplatin, methotrexate, vinblastine, doxorubicin, paclitaxel, carboplatin, adriamycin, gemcitabine, filgramastim, pemetrexed, vinorelbine, oxaliplatin, vinflunine, or doxetaxel.

In a related embodiment, the invention provides additional T cell-stimulating pharmaceutical formulations for use in the treatment of patients classified as antineoplastic treatment responders according the methods described above. These include, but are not limited to, Bacillus Calmette-Guérin (BCG), the related increased safety recombinant strain VPM1002 (Kaufmann Frontiers Immunol. 2020, 11:316; Grode et al., Vaccine 2013, 31(9):1340) and cancer immunotherapy treatments, particularly checkpoint inhibitory antibodies, more particularly a checkpoint inhibitor antibody selected from ipilimumab, nivolumab, pembrolizumab, pidilizumab, atezolizumab, avelumab, durvalumab, or cemiplimab.

The data provided in the examples shows that these biomarkers may be particularly helpful for predicting whether a patient will respond well to an antineoplastic immunomodulatory medication that increases T cell killing of tumour cells, included checkpoint blockade or BCG treatment, as the biomarker levels are directly correlated with the number, and activation, or functional phenotype of T cells in the tumour. Studies in mouse models of melanoma and ovarian cancer have suggested that the outcome of immune checkpoint blockade correlates with certain components of the CXCR3 chemokine signals in tumours (Chow N. T. et al. Immunity (2019), 50(6):1498). The human data in MBIC provided shows the unexpected predictive efficacy of CXCL11 and CXCR3A or CXCR3alt in predicting T cell activation in neoadjuvant chemotherapy, suggesting the methodology would be successful in similar treatment options which rely on T cell immunity.

A further aspect of the invention provides a system for assigning a value to a patient tissue sample that reflects the T cell activity state of the tissue, comprising determining the expression level in the tissue sample of the genes CXCL11, CXCR3A and/or CXCR3alt, and optionally CXCL4, CXCL9, CXCL10 and CXCR3B, and using the biomarker expression levels to classify the T cell activity state of the patient sample.

The invention is further illustrated by the following items:
V. A method to measure the T cell activity state in a patient tissue sample, wherein the method comprises the steps of:
   a. providing a patient tissue sample;
   b. in a measurement step, determining a biomarker expression level of the biomarkers
      CXCL11 and at least one of CXCR3A or CXCR3alt,
         and optionally, CXCR3B, CXCL4, CXCL9 and/or CXCL10,
   c. in an optional classification step, assigning the tissue sample a value which reflects the number of activated T cell present in the patient tissue sample, based on the biomarker expression levels.
W. A method to measure the T cell activity state in a cancer tissue, wherein the method comprises the steps of:
   a. providing a cancer tissue sample;
   b. in a measurement step, determining a biomarker expression level of the biomarkers
      CXCL11 and at least one of CXCR3A or CXCR3alt,
      and optionally, CXCR3B, CXCL4, CXCL9 and/or CXCL10;
   c. in an optional classification step, assigning the cancer tissue sample a value which reflects the number of activated T cells present in the cancer tissue sample, based on the biomarker expression levels.
X. A method of predicting the outcome of an antineoplastic treatment in a patient bearing a solid tumour, wherein the method comprises the steps of:
   a. providing a cancer tissue sample;
   b. in a measurement step, determining a biomarker expression level of the biomarkers
      CXCL11 and at least one of CXCR3A or CXCR3alt,
         and optionally, CXCR3B, CXCL4, CXCL9 and/or CXCL10;
      c. in a classification step, assigning the patient to a predicted treatment outcome based on the biomarker expression levels.
Y. A method to measure the T cell activity state in a tumour, or of predicting the outcome of an antineoplastic drug treatment in a patient bearing a solid tumour, wherein the method comprises the steps of:
   a. providing a cancer tissue sample;
   b. in a measurement step, determining a biomarker expression level of at least one of the biomarkers: CXCL4, CXCL9, CXCL10, CXCL11, CXCR3A, CXCR3alt, and/or CXCR3B, particularly determining the expression level of CXCL11 and/or CXCR3alt;
   c. in an optional classification step, assigning the cancer tissue sample a value which reflects the number of activated T cells present in the cancer tissue sample, or assigning the patient to a predicted treatment outcome based on the biomarker expression levels.
Z. The method according to item Y, wherein in the measurement step, the expression level of at least two biomarkers is determined, particularly wherein the expression level of more than three biomarkers selected from CXCL4, CXCL9, CXCL10, CXCL11, CXCR3A, CXCR3alt, and/or CXCR3B is determined.
AA. The method according to any one of the items V to Z, wherein in the measurement step, the expression level of the biomarkers CXCL11 and CXCR3alt are determined.
BB. The method according to any one of the items X to AA, wherein the classification step comprises inputting the biomarker expression levels into an algorithm to provide a probability that a patient will be an antineoplastic treatment responder.
CC. The method of predicting the outcome of an antineoplastic treatment in a patient bearing a solid tumour according to any of the items X to AA, wherein the classification step comprises:
   classifying the cancer patient as an antineoplastic treatment responder if the biomarker expression level is above or below a threshold, or
   classifying the cancer patient as an antineoplastic treatment non-responder if the biomarker expression level is above or below a threshold.
DD. The method according to any one of the items V to CC, wherein the expression level of CXCL11 is determined at the protein level, and wherein the expression level of CXCR3A, CXCR3alt and/or CXCR3B is determined at the mRNA level.
EE. The method according to item DD, wherein the expression level of CXCR3B is determined using a set of nucleic acid primers comprising, or essentially consisting of, the sequences SEQ ID NO 016, SEQ ID NO 017, and SEQ ID NO 018, and/or
   the expression level of CXCR3alt is determined using a set of nucleic acid primers comprising, or essentially consisting of, the sequences SEQ ID NO 019, SEQ ID NO 020 and SEQ ID NO 021.
FF. The method of predicting the outcome of an antineoplastic treatment in a patient bearing a solid tumour according to any of the items X to EE, wherein the classification step comprises:
   classifying the cancer patient as an antineoplastic treatment responder if the level of expression of the biomarkers is above an expression level in a negative reference sample, and/or equal or above an expression level in a positive reference sample, or
   classifying the cancer patient as an antineoplastic treatment non-responder if the level of expression of the biomarkers is below an expression level in a positive reference sample, and/or equal or below an expression level in a negative reference sample.
GG. The method according to any of the items V to FF, wherein the expression level of CXCL11 is normalised, particularly wherein the expression level of CXCL11 is normalised to the mass of the sample.
HH. The method of predicting the outcome of an antineoplastic treatment in a patient bearing a solid tumour according any of the items X to GG, wherein in the cancer patient is classified as an antineoplastic treatment responder if the expression level of CXCL11 is more than 13.98 pg per 10 mg sample tissue, particularly more than. 22.4 pg per 10 mg sample tissue.
II. The method to measure the T cell activity state, or predict the outcome of an antineoplastic treatment in a patient bearing a solid tumour of according to any of the items V to HH, wherein the biomarker expression level is normalised to the expression level of at least one housekeeping (HKG) gene, particularly HKG IP08 and/or CDKN1B.
JJ. The method according to item II, wherein in the classification step, assigning the cancer patient as an antineoplastic treatment responder if the fold change of the expression level of
   CXCR3A is more than 2^{(-12.3)} more than (>) the HKG expression level, particularly >2^{(-11.97)} times the HKG expression level, and/or
   CXCR3alt is more than 2^{(-13.8)}>the HKG expression level, particularly more than 2^{(-11.27)} times the HKG expression level, and/or
   CXCR3B is more than 2^{(-11.9)}>the HKG expression level, particularly more than 2^{(-8.43)} times the HKG expression level.
KK. The method according to any of the items W to JJ, wherein the cancer tissue sample comprises or essentially consists of, neoplastic cells derived from a squamous cancer or adenocarcinoma, more particularly neoplastic cells derived from kidney, prostate, breast, lung, ovarian, gastric, rectal, melanoma, oesophageal or bladder cancer.
LL. The method according to any of the items W to KK, wherein the cancer patient is a bladder cancer patient, particularly a patient having been diagnosed with muscle-invasive bladder cancer.
MM. The method according to any of the items X to LL, wherein the antineoplastic treatment is selected from:
   an antineoplastic drug, particularly an antineoplastic drug selected from cisplatin, methotrexate, vinblastine, doxorubicin, carboplatin, adriamycin, gemcitabine, paclitaxel, filgramastim, pemetrexed, vinorelbine, oxaliplatin, vinflunine, or doxetaxel,
   a cancer immunotherapy, particularly a checkpoint inhibitory antibody, more particularly a checkpoint inhibitor antibody selected from ipilimumab, nivolumab, pembrolizumab, pidilizumab, atezolizumab, avelumab, durvalumab, or cemiplimab,
   Bacillus Calmette-Guérin, or VPM1002.
NN. The method according to any of the items W to MM, wherein the sample is tumour tissue, peripheral blood, white blood cells, or lymph node tissue, particularly wherein the sample is a tumour biopsy, or draining lymph node tissue from the vicinity of the tumour.
OO. The method to predict the clinical outcome of cancer patient according to any of the items X to NN, wherein cancer patient has been classified as an antineoplastic treatment non responder, and wherein a higher likelihood of clinical benefit resultant from treatment comprising resection of malignant tissue compared with antineoplastic drug treatment is assigned to the cancer patient.
PP. The method to predict the clinical outcome of cancer patient according to item OO, wherein the cancer is muscle invasive bladder cancer, and wherein the resection of malignant tissue is a radical cystectomy procedure.
QQ. A method to predict the outcome of a tissue targeted immune cell transfer treatment, particularly a T cell transfer treatment for cancer in a patient bearing a solid tumour, wherein the method comprises the steps of:
   a. providing a cell transfer sample, particularly recombinant or expanded TILs;
   b. providing a target tissue sample from a patient, particularly a tumour sample from a patient bearing a solid tumour;
   c. in a cell transfer measurement step, determining the expression level of at least one of the biomarkers CXCR3A, CXCR3alt, and/or CXCR3B;
   d. in a target tissue sample measurement step, determining the expression level of at least one of the biomarkers CXCL4, CXCL9, CXCL10, and/or CXCL11;
   e. in a classification step, assigning the patient a likelihood that the tissue targeted immune cell transfer treatment will have a positive clinical outcome based on the biomarker expression levels.
RR. A method to monitor a response to antineoplastic treatment in a patient bearing a solid tumour, wherein the method comprises the steps of
   a. providing a pre-treatment cancer tissue sample obtained from the cancer patient before the onset of an antineoplastic treatment, and
   b. providing a post-treatment cancer tissue sample obtained after the onset of the antineoplastic treatment;
   c. in a measurement step, determining a biomarker expression level of the biomarkers
      CXCL11 and
      at least one of CXCR3A or CXCR3alt,
      and optionally, CXCR3B, CXCL4, CXCL9 and/or CXCL10;
   d. in a classification step, classifying the patient as an antineoplastic treatment responder if the level of biomarker expression in the post treatment sample is above the level of biomarker expression of the pre-treatment sample.
SS. A pharmaceutical compound selected from:
   an antineoplastic drug, particularly an antineoplastic drug selected from cisplatin, methotrexate, vinblastine, doxorubicin, carboplatin, adriamycin, gemcitabine, paclitaxel, filgramastim, pemetrexed, vinorelbine, oxaliplatin, vinflunine, or doxetaxel,
   a cancer immunotherapy, particularly a checkpoint inhibitory antibody, more particularly a checkpoint inhibitor antibody selected from ipilimumab, nivolumab, pembrolizumab, pidilizumab, atezolizumab, avelumab, durvalumab, or cemiplimab,
   Bacillus Calmette-Guérin
   for use in the treatment of a patient with a solid tumour, wherein the pharmaceutical compound is administered to a patient having been classified as an antineoplastic treatment responder by a method according to any of the items V to NN.
TT. A system to T cell activity state in a tissue sample, wherein the method comprises the steps of:
   a. providing a tissue sample;
   b. in a measurement step, determining a biomarker expression level of at least one of the biomarkers:
      i. CXCL4, CXCL9, CXCL10, CXCL11, CXCR3A, CXCR3alt, and/or CXCR3B;
      ii. particularly determining the expression level of CXCL11 and/or CXCR3alt,
   c. in a classification step, assigning the tissue sample a value which reflects the T cell activity state present in the tissue sample, based on the biomarker expression levels.
UU. A method to measure the T cell activity state in a patient tissue sample, particularly in a cancer tissue sample, or to predict the outcome of an antineoplastic treatment in a patient bearing a solid tumour, wherein the method comprises the steps of:
   a. providing a patient tissue sample, particularly a cancer tissue sample;
   b. in a measurement step, determining a biomarker expression level of the biomarkers
      CXCL11 and
      at least one of CXCR3A or CXCR3alt,
         and optionally, CXCR3B, CXCL4, CXCL9 and/or CXCL10;
   c. in an optional classification step,
      assigning the tissue sample a value which reflects the number of activated T cell present in the patient tissue sample, or the cancer tissue sample, or
      assigning the patient to a predicted treatment outcome,
   based on the biomarker expression levels.
VV. A method to measure the T cell activity state in a tumour, or of predicting the outcome of an antineoplastic drug treatment in a patient bearing a solid tumour, wherein the method comprises the steps of:
   a. providing a cancer tissue sample;
   b. in a measurement step, determining a biomarker expression level of at least one of the biomarkers:
      CXCL4, CXCL9, CXCL10, CXCL11, CXCR3A, CXCR3alt, and/or CXCR3B particularly determining the expression level of CXCL11 and/or CXCR3alt;
   c. in an optional classification step, assigning the cancer tissue sample a value which reflects the number of activated T cells present in the cancer tissue sample, or assigning the patient to a predicted treatment outcome based on the biomarker expression levels.
WW. The method according to item VV, wherein in the measurement step, the expression level of at least two biomarkers is determined, particularly wherein the expression level of more than three biomarkers selected from CXCL4, CXCL9, CXCL10, CXCL11, CXCR3A, CXCR3alt, and/or CXCR3B is determined.
XX. The method according to any one of the items UU to WW, wherein in the measurement step, the expression level of the biomarkers CXCL11 and CXCR3alt are determined.
YY. The method of predicting the outcome of an antineoplastic treatment in a patient bearing a solid tumour according to any of the items UU to XX, wherein the classification step comprises:
   classifying the cancer patient as an antineoplastic treatment responder if the biomarker expression level is above or below a threshold, or
   classifying the cancer patient as an antineoplastic treatment non-responder if the biomarker expression level is above or below a threshold.
ZZ. The method according to any one of the items UU to YY, wherein the expression level of CXCL11 is determined at the protein level, and wherein the expression level of CXCR3A, CXCR3alt and/or CXCR3B is determined at the mRNA level.
AAA. The method of predicting the outcome of an antineoplastic treatment in a patient bearing a solid tumour according any of the items UU to ZZ, wherein in the cancer patient is classified as an antineoplastic treatment responder if the expression level of CXCL11 is more than 13.98 pg per 10 mg sample tissue, particularly more than. 22.4 pg per 10 mg sample tissue.
BBB. The method according to any one of the items UU to AAA, wherein the expression level of CXCR3A, CXCR3alt and/or CXCR3B is normalised to the expression level of at least one housekeeping gene (HKG), particularly the HKG IP08 and/or CDKN1B, and wherein in the classification step the cancer patient is assigned as an antineoplastic treatment responder if the fold change of the expression level of
   CXCR3A is more than 2^{(-12.3)} more than (>) a (HKG) expression level, particularly >2^{(-11.97)} times the HKG expression level, and/or
   CXCR3alt is more than 2^{(-11.9)}>the HKG expression level, particularly more than 2^{(-11.27)} times the HKG expression level, and/or
   CXCR3B is more than 2^{(-11.9)}>the HKG expression level, particularly more than 2^{(-9.43)} times the HKG expression level.
CCC. The method according to any of the items UU to BBB, wherein the cancer tissue sample comprises or essentially consists of, neoplastic cells derived from a squamous cancer or adenocarcinoma, more particularly neoplastic cells derived from kidney, prostate, breast, lung, ovarian, gastric, rectal, melanoma, oesophageal or bladder cancer.
DDD. The method according to any of the items UU to CCC, wherein the cancer patient is a bladder cancer patient, particularly a patient having been diagnosed with muscle-invasive bladder cancer.
EEE. The method according to any of the items UU to DDD, wherein the antineoplastic treatment is selected from:
   an antineoplastic drug, particularly an antineoplastic drug selected from cisplatin, methotrexate, vinblastine, doxorubicin, carboplatin, adriamycin, gemcitabine, paclitaxel, filgramastim, pemetrexed, vinorelbine, oxaliplatin, vinflunine, or doxetaxel,
   a cancer immunotherapy, particularly a checkpoint inhibitory antibody, more particularly a checkpoint inhibitor antibody selected from ipilimumab, nivolumab, pembrolizumab, pidilizumab, atezolizumab, avelumab, durvalumab, or cemiplimab.
   Bacillus Calmette-Guérin, or VPM1002.
FFF. The method according to any of the items UU to EEE, wherein the sample is tumour tissue, peripheral blood, white blood cells, or lymph node tissue, particularly wherein the sample is a tumour biopsy, or draining lymph node tissue from the vicinity of the tumour.
GGG. The method according to any of the items UU to FFF, wherein cancer patient has been classified as an antineoplastic treatment non responder, and wherein a higher likelihood of clinical benefit resultant from treatment comprising resection of malignant tissue compared with antineoplastic drug treatment is assigned to the cancer patient, particularly wherein the cancer is muscle invasive bladder cancer, and wherein the resection of malignant tissue is a radical cystectomy procedure.
HHH. A method to monitor a response to antineoplastic treatment in a patient bearing a solid tumour, wherein the method comprises the steps of
   a. providing a pre-treatment cancer tissue sample obtained from the cancer patient before the onset of an antineoplastic treatment, and
   b. providing a post-treatment cancer tissue sample obtained after the onset of the antineoplastic treatment;
   c. in a measurement step, determining a biomarker expression level of the biomarkers
      CXCL11
      and
      at least one of CXCR3A or CXCR3alt,
         and optionally, CXCR3B, CXCL4, CXCL9 and/or CXCL10;
   d. in a classification step, classifying the patient as an antineoplastic treatment responder if the level of biomarker expression in the post treatment sample is above the level of biomarker expression of the pre-treatment sample.
III. A pharmaceutical compound selected from:
   an antineoplastic drug, particularly an antineoplastic drug selected from cisplatin, methotrexate, vinblastine, doxorubicin, carboplatin, adriamycin, gemcitabine, paclitaxel, filgramastim, pemetrexed, vinorelbine, oxaliplatin, vinflunine, or doxetaxel,
   a cancer immunotherapy, particularly a checkpoint inhibitory antibody, more particularly a checkpoint inhibitor antibody selected from ipilimumab, nivolumab, pembrolizumab, pidilizumab, atezolizumab, avelumab, durvalumab, or cemiplimab.
   Bacillus Calmette-Guérin
for use in the treatment of a patient with a solid tumour, wherein the pharmaceutical compound is administered to a patient having been classified as an antineoplastic treatment responder by a method according to any of the items UU to FFF.

The invention is further illustrated by the following examples and figures, from which further embodiments and advantages can be drawn. These examples are meant to illustrate the invention but not to limit its scope.

### DESCRIPTION OF THE FIGURES

FIG. 1 shows flow cytometric analysis of ex-vivo chemokine receptor expression on CD8⁺ T cell subpopulations derived from healthy donor peripheral blood mononuclear cells (a-b, PBMC n=11), or lymph node samples from muscle-invasive bladder cancer (MIBC)-patients undergoing radical cystectomy (c-e, LN). a, flow cytometry of a representative human PBMC sample: naïve (TNANE: CCR7⁺CD45RA⁺), memory stem (T_{SCM}: CCR7⁺CD45RA⁺; CD95⁺), central memory (T_{CM}: CCR7⁺CD45RA⁻), effector memory (TEM: CCR7⁻CD45RA⁻) and terminally differentiated effector T cells (T_{EMRA}: CCR7⁻CD45RA⁺). b, quantification of mean fluorescence intensity (MFI) of ex-vivo expression of the indicated chemokine receptors in healthy human PBMC (n=9), median values and range of each population in a, respectively. c, Representative FACS-plots and quantification of CD8⁺ T cell subpopulations in PBMC and LN-derived lymphocytes from PBMC and LN samples from MIBC patients (n=5, LN are median values of 1-3 nodes per patient). Two-tailed paired t-test. *P≤0.05. d, Representative histograms of CXCR3 expression on CD8⁺ T cell subpopulations in PBMC and lymphocytes taken from one LN of one BC-patient. e, Quantification of the concentration of CXCL9/10/11 in serum and bulk LN homogenates measured by Luminex, n=3 MIBC patients, mean with SEM; Friedmann test with Dunn's post-test *P≤0.05;0.01; ***P≤0.001.

FIG. 2 a, Quantification of flow cytometry of the frequencies of activation of LN CD8⁺ T cells from muscle-invasive bladder cancer (MIBC) patients undergoing radical cystectomy, stimulated with matched tumour cells. LN cells were stimulated for 12 h with autologous bladder tumour lysates and activation was measured by increased CD137 expression. Gating as in FIG. 1c. n=7 LN from 3 MIBC patients, mean and SEM are shown. Paired t-test *P≤0.05; **P≤0.01. b, Heat map of background-normalized CD137 expression in response to autologous bladder tumour lysates by LN and PBMC CD8⁺ T cells stimulated as above. Colour indicates ΔCD8⁺CD137⁺ higher than background activation.

FIG. 3 shows a, in-vitro chemotaxis to CXCR3-ligands CXCL9/10/11 by CD8⁺ T cells, using flow cytometry and calculating the chemotactic index (CI) relative to the absolute number of migrated cells in the lower chamber without chemokine. CI>1 (dashed line) indicates a chemotactic effect of the ligand. Median values are shown. b, Quantification of the paired frequency of CD8+CXCR3+ T cell expression in the upper (non-migrated) and the lower (migrated) chamber of a CXCL9 migration assay.

FIG. 4 shows expansion of CMV-specific CD8+ T cells from purified naïve T cells +/− CXCL9/10/11 (n=6 healthy donors). a, Representative flow cytometry of CMV-induced activation at day 14 (upper panel) and day 21 (lower panel). b, Fold enrichment of the frequencies of CMV-specific CD8+CCR7⁺CD45RA⁺-derived T_{SCM} expanded +/− CXCL9/10/11 according to b. c, Quantification of the frequency of CMV-induced IFN-γ+ cells within CD8+CCR7+CD45RA+-derived T cells expanded in +/− CXCL11 for 14 days. Wilcoxon rank-sum *P50.05 d, Representative flow cytometry of CXCR3 expression on CD8+ CMV-specific CCR7+CD45RA+-derived T cells on day 21 of CMV_{IE-1/pp65}-overlapping peptide pool pulsed LCL. e, Representative flow cytometry (left) or quantification of the frequency of cell division in CXCL11 stimulated CD8+ T_{SCM} cells. Mean values and SEM are shown. f, mRNA CXCR3 variant (CXCR3A, CXCR3B and CXCR3alt) expression in distinct CD8+ T cell subsets in PBMC, and a model of CXCR3 variant specific interactions (CXCR3A/B/alt) with CXCR3-ligands (CXCL9/10/11). FAC sorting according to FIG. 1a. CXCR3 isoform expression measured by qPCR was normalised to the housekeeping gene (HKG) HPRT (CXCR3 isoform/HKG). Mean values and SEM are shown, healthy donors n=5; One-way ANOVA *P≤0.05; *P≤0.01; *P≤0.001.

FIG. 5 shows clinical stratification of BC patients and pre-treatment analysis of the intra-tumoural T cell levels. a, Workflow of the study on bladder cancer (BC) patients and clinical stratification of platinum-based neoadjuvant chemotherapy (NAC) before radical cystectomy (RC) for muscle-invasive bladder cancer (MIBC) patients. The clinical workflow integrates pre-treatment tumour sampling via TURBT (transurethral resection of bladder tumour) to assign patients to MIBC vs. non-MIBC (NMIBC). Clinically eligible MIBC-patients were treated with NAC. Post-NAC, MIBC-patients were identified as responder (resp.) by pathoanatomical downstaging in the tumour histology of the radical cystectomy specimen and as non-responder (non-resp.) with stable or progressive disease. b, Kaplan-Meier-estimate of overall survival (%) (NMIBC, NAC-receiving MIBC, no-NAC MIBC). Patients at risk are indicated below the graph. c, CD3 mRNA expression was measured by in the treatment-naïve tumour samples of BC patients by qPCR. Mean of IPO8 and CDKN1B (HKGs) were used for normalisation: fold of delta CT CD3 per delta CT HKG. Black lines in violin plots show median. Mann-Whitney test **P≤0.01. Kaplan-Meier-estimate of overall survival (%) showing splitting NAC-receiving MIBC patients into CD3-high (n=10) and CD3-low (n=10) gives individual Cox proportional hazard model fits *P≤0.05; Likelihood-ratio test for responder (resp., n=9) and non-responders (non-resp., n=11). Retrospective observation time for KM curves was 6 years. In all graphs, NMIBC-patients (n=17), no-NAC patients (n=9) and NAC-receiving patients (n=20) separated into resp. (n=9) and non-resp. (n=11) patients is shown.

FIG. 6 shows intra-tumoural cytokine milieus differ between BC-patient groups. Nonmetric multidimensional scaling (NMDS) of 46 BC-patient samples is shown according to their dissimilarities in the expression levels of 75 chemotactic cytokines measured by multiplex ELISA. Ellipses enclose individual group masses and arrows indicate average contribution of each cluster of cytokines to the ordination.

FIG. 7 shows CXCL11 is a biomarker for NAC-responsive MIBC-patients associated with intra-tumoural T cell levels. ELISA-based multiplex-detection was used to measure intra-tumoural cytokines in primary biopsies of the BC-patient cohort (Tab. 1, n=46). a, Receiver operating characteristic (ROC) curves show biomarker prediction of positive NAC outcome (=response) listed as sensitivity and prediction of negative outcome of NAC (=non-response) listed as specificity. The capacity of each marker to estimate the clinical outcome including the prediction of NAC-response in MIBC and overall survival (OS) was tested. Sensitivity and specificity are shown as frequency setting 100% as correct prediction. Shading indicates the superior efficacy of CXCL11 as a biomarker over all others tested. b, Intra-tumoural CXCL11 protein-levels of the BC-subgroups (NMIBC, no-NAC MIBC, NAC-receiving MIBC including resp. & non-resp.) measured by multiplex ELISA. Black lines show median. Kruskal-Wallis with Dunn's post-test. **P≤0.01. c, Linear correlation analysis between intra-tumoural CXCL11 protein and CD3 mRNA of NAC-receiving MIBC-patients. Resp. shown as full shaded dots, non-resp. as open dots. Spearman R=0.65 and P=0.0021. d, Kaplan-Meier curves show OS of MIBC patients split into CXCL11 high (n=11) and CXCL11 low (n=9) level-groups. Improved survival outcome of NAC-receiving MIBC patients with higher intra-tumoural CXCL11 protein levels as determined by Cox regression (*P≤0.05; Likelihood-ratio test). NMIBC-patients (n=17), no-NAC patients (n=9) and NAC-receiving patients (n=20) separated into resp. (n=9) and non-resp. (n=11) patients is shown.

FIG. 8 shows CXCR3 expression associates to tissue-infiltrating CD8+ T cells in healthy bladder and MIBC. A t-SNE map generated from combined single-cell RNA-sequencing samples datasets from 2 MIBC samples (4080 cells) and of 3 healthy bladder samples (13440 cells). Individual cells are coloured by origin (a), normalized log-expression levels of CD8 and CXCR3 (b), which colocalize in immune cell-specific clusters (c).

FIG. 9 shows CXCR3 expression associates to CD8⁺ tumour infiltrating T cells and CXCL9/10/11 expression to macrophages in melanoma. Single-cell RNA-sequencing data of melanoma samples accessed from 19 different patients (total of 3187 cells). T-SNE maps showing a, cell-type classification. b, Log-normalized single-cell expression levels indicate CD8 and CXCR3 expression confined to T cells, and c, abundant expression of CXCL9, CXCL10, and CXCL11 in the CD14+ cluster of monocytes/macrophages.

FIG. 10 shows mRNA-level of CXCR3 isoforms measured by qPCR from primary biopsies of the Swedish BC-patient cohort. a, Linear correlation analysis between intra-tumoural CXCR3-variants and CD3 mRNA of NAC-receiving MIBC-patients. b, ROC-curves demonstrate the ability of CXCR3A, CXCR3B and CXCR3alt to predict positive outcome of NAC (=response) listed as sensitivity and to predict negative outcome of NAC (=non-response) listed as specificity. Sensitivity and specificity are shown as frequency, setting 100% as a correct prediction. Shaded area indicates superior efficacy (highest AUC) of CXCR3alt. c, Intra-tumoural mRNA-levels of CXCR3alt from the patient subgroups analysed by qPCR. NAC-receiving MIBC are subdivided according to the pathological response on NAC in resp. and non-resp. Mean of IPO8 and CDKN1B (HKGs) were used for normalization: fold of delta CT CD3 per delta CT HKG. Kruskal-Wallis with Dunn's post-test. **P≤0.01; ***P≤0.001. Black lines in violin plots show median. d, Ratio of Intra-tumoural CXCR3alt-CD3 mRNA-levels from the patient subgroups. mRNA expression of CXCR3alt was analysed by qPCR, according to c. e, Effect of intra-tumoural CXCR3alt mRNA-levels on OS of NAC-receiving MIBC patients as indicated by optimal split into CXCR3alt-high (n=12) and CXCL3alt-low (n=8) of individual Cox proportional hazard model fits (*P≤0.05; Likelihood-ratio test). NMIBC-patients (n=17), no-NAC patients (n=9) and NAC-receiving patients (n=20) separated into resp. (n=9) and non-resp. (n=11) patients is shown.

FIG. 11 upper panel shows partial correlation network analysis of a cluster of 14 biomarkers of intra-tumoural cytokines measured by ELISA based multiplex in tumour protein lysate and CXCR3 isoforms measured by qPCR, and the NAC-response to highlight independently predictive variables in MIBC patients. Lower panel shows dual stratification with CXCL11 and CXCR3alt predicts the response to NAC in MIBC. Synergistic effects of the intra-tumoural receptor CXCR3alt and its ligand CXCL11 on the NAC-response were assessed by logistic regression analysis for prediction (AUC=1, LOOCV accuracy=0.9). Shading indicates predicted probability of response/no response.

FIG. 12 shows Intra-tumoural CXCL11^{hi} mRNA predicts improved overall survival in MIBC patients from the TCGA cohort. (a) Kaplan-Meier estimates show overall survival (OS) of MIBC-patients receiving chemotherapy (chemo) and non-chemotherapy treated MIBC-patients (non-chemo). (b) Linear correlation analysis between intra-tumoural mRNA-levels of CXCR3, CXCL9/10/11, and CD3 of chemotherapy-receiving MIBC-patients (top panels) and non-treated MIBC-patients (bottom panels). (c) Heat Map of Spearman's rank-correlation coefficients (including CD8). (d) Kaplan-Meier curves show OS of chemotherapy-receiving MIBC patients stratified by mRNA expression levels. Data were dichotomized using optimal split points into CXCL9high (n=21) and CXCL9low (n=47), CXCL10high (n=28) and CXCL10low (n=40), CXCL11high (n=13) and CXCL11low (n=55), and CXCR3high (n=48) and CXCR3low (n=20) and CXCL4low (n=56) and CXCL4high (n=12).

Tab. 1 shows the protein analytes measured by multiplex ELISA using Luminex.

Tab. 2 shows the performance of individual biomarker thresholds for predicting the clinical outcome of neoadjuvant therapy treatment in NMIBC patients. Decreasing AIC or Brier scores indicated a better model fit.

Tab. 3 shows the performance of logistic regression models for predicting the clinical outcome of neoadjuvant therapy treatment in NMIBC patients.

Tab. 4 Table shows hazard ratios and coefficients of the MIBC patients from the TCGA cohort (n=68). Data were dichotomized using optimal split points into CXCL9high (n=21) and CXCL9low (n=47), CXCL10high (n=28) and CXCL10low (n=40), CXCL11high (n=13) and CXCL11low (n=55), and CXCR3high (n=48) and CXCR3low (n=20) patients, and CXCL4low (n=56) and CXCL4high (n=12).

### EXAMPLES

### Methods

### Patients

46 patients with BC were recruited with informed consent from different hospitals in the northern health region of Sweden during the years 2010-2017. Specimens and blood samples were archived in the biobank of the department of urology at the university hospital in Umeå (NUS), Sweden. Patients were at least 18 years of age, and the study on patient material was approved by the regional ethical board (EPN-Umeå, original registration number: 2013/463-31M, with latest amendment 2018/545-32). Further, all patients had given verbal and written consent to contribute with specimens and fluids to the biobank and to participate in consecutive and ethically approved translational research. A second cohort of normalized mRNA expression data of primary tumour samples was obtained from The Cancer Genome Atlas (TCGA, https://portal.gdc.cancer.gov). Clinical data (BLCA dataset) were used to identify 68 MIBC patients that received chemotherapy (chemo) within 150 days after sample procurement and 292 MIBC-patients that did not receive any chemotherapy (no-chemo).

Diagnosis and NAC-Treatment

The diagnosis of urinary BC was established based on tumour histology of the specimen that was received at transurethral resection of the bladder tumour (TURBT). In the TURBT sample, MIBC disease was defined by the histological invasion of the tumour into the detrusor muscle; cT2-T4 (29/46 patients). Next, MIBC-patients were clinically investigated on eligibility to receive NAC containing based on a good performance status including Charleson age comorbidity index (CACI) ≤6, age≤77 years and no major renal impairment (GFR≥55-60) or any other relevant comorbidity. NAC treatment in most cases contained a high dose of the drugs methotrexate, vinblastine, doxorubicin or epirubucin, and cisplatin according to the following regime:
Day 1 Methotrexate 30 mg/m²
Day 2 Vinblastin 3 mg/m², Doxorubicin 30 mg/m², Cisplatin 70 mg/m² (max. 140 mg)
Day 3 Pegfilgrastim 6 mg sub cutaneous

Further, by radiological computer tomography (CT) nodal and organ-dissemination was excluded; cN0M0³⁵². Eligible MIBC-patients (20/29) received 2-4 cycles of NAC-treatment before radical surgery (i.e. cystectomy with radical intention: RC). NAC was applied as Cisplatin-based combination chemotherapy (predominantly: cisplatin, methotrexate, vinblastine, doxorubicin (MVAC). Response to NAC was defined as pathoanatomical downstaging of the tumour in the RC-specimen and based on this, NAC-receiving MIBC patients were defined as responders (9) or non-responders (11). These two groups had equivalent clinical performance status exemplified by similar ranges in the CACI index, the American Society of Anaesthesiologists Classification (ASA)-score and patient age. Further, response to NAC was subdivided based on the tumour histology into complete response (CR) with p0N0M0, partial response (PR) with pTa/T1/TisN0M0, stable disease (SD) with ≥p2N0M0 and progressive disease (PD) with any pT and N1/2 and/or M1. 5 patients exhibited CR, 4 patients exhibited PR, 4 patients exhibited SD and 7 patients exhibited PD. 7/29 MIBC-patients were ineligible for NAC (i.e. no-NAC MIBC patients; see criteria above) and underwent direct RC (4/7) or due to palliative reasons, RC was not applied (3/7). If the tumour infiltration in TURBT-specimen was limited to the subepithelial or epithelial layer, the tumour was defined as non-muscle invasive bladder cancer (NMIBC). NMIBC patients underwent non-systemic treatment such as local administration of Bacillus Calmette-Guérin (BCG) vaccine and when indicated, re-TURBT treatment.

Patient Sample Processing

The tumour samples were taken during TURBT and lymph nodes were taken during RC. All specimens were immediately frozen in liquid nitrogen and stored at −80° C. For processing, specimens were kept on ice at all times, cut in two parts with a scalpel and the mass was scaled. Next, protein extraction buffer (T-PER™; Thermo Fisher Scientific) was applied to one part and RNA/DNA lysis buffer (RLT; Quiagen) with 2 M DTT was applied to the other part. Specimens were mechanically disrupted using tubes with ceramic beads in a tissue homogenizer system (all from Bertin Instruments). Concomitant DNA/RNA extraction was performed using the AllPrep DNA/RNA Micro Kit following the manufacturer's instructions (Quiagen). 13 lymph nodes were kept non-disrupted after RC in order to isolate live lymphocytes. After immersion in cold AIM-V medium (Thermo Fisher Scientific), the specimen was cut with a scalpel and cells were gently filtered through a 40 μM cell strainer.

PBMC Preparation

Blood samples were collected from healthy volunteers after obtaining informed consent. Human peripheral blood mononuclear cells (PBMCs) were separated from the heparinized whole blood of healthy donors by lymphoprep density gradient centrifugation with a Biocoll separating solution (Biochrom GmbH, Berlin). Isolated PBMC were re-suspended in PBS and kept at 4° C. The study on PBMC was approved by the Charité University Medical School Ethical Committee (institutional review board).

Flow Cytometric Analysis

For analysis of T cell phenotypes, PBMCs and lymph node-derived cells were stained using fluorescently conjugated monoclonal antibodies for CD3 (BV650, clone OKT3), CD4 (PerCP-Cy5.5, clone SK3), CD8 (BV570, clone RPA-T8), CCR7 (AF647, clone G043H7), CD45RA (PE/Dazzle 594, clone HI100), and CD95 (PE/Cy7/Brilliant Violet (BV) 421, clone DX2; BD Biosciences), CXCR3 (PE, clone G025H7) at 4° C. for 30 min. To exclude dead cells, LIVE/DEAD Fixable Blue Dead Cell Stain dye (Thermo Fisher Scientific) was added. Analogously, chemokine receptors (CXCR1, CXCR3, CXCR4, CCR3, CCR5, CCR6, CCR7) were stained on the cell surface using the human cell surface marker screening panel (BD Biosciences). All antibodies were purchased from BioLegend, unless otherwise indicated. Cells were analysed on an LSR-II FORTESSA flow cytometer (BD Biosciences) and FlowJo software version 10 (Tree Star). Lymphocytes were gated on the basis of the forward scatter (FSC) versus side scatter (SSC) profile and subsequently gated on FSC-Height versus FSC-Area to exclude doublets. In stimulation experiments, fixation/permeabilization was performed with an eBioscience FoxP3/Transcription Factor Staining Buffer Set (Thermo Fisher Scientific) according to the manufacturer's instructions. After washing, fixed cells were stained with the fluorochrome-conjugated monoclonal antibodies for IFN-γ (eF405, clone 4S.B3), for TNF-α (Alexa Fluor 700, clone MAb11) and for CD137 (PE/Cy7, clone 4B4-1) at 4° C. for 30 min. Background response was assessed using non-stimulated controls and subtracted from the antigen-reactive cytokine production.

Chemotaxis Assay for CD8+ T Cell Subpopulations.

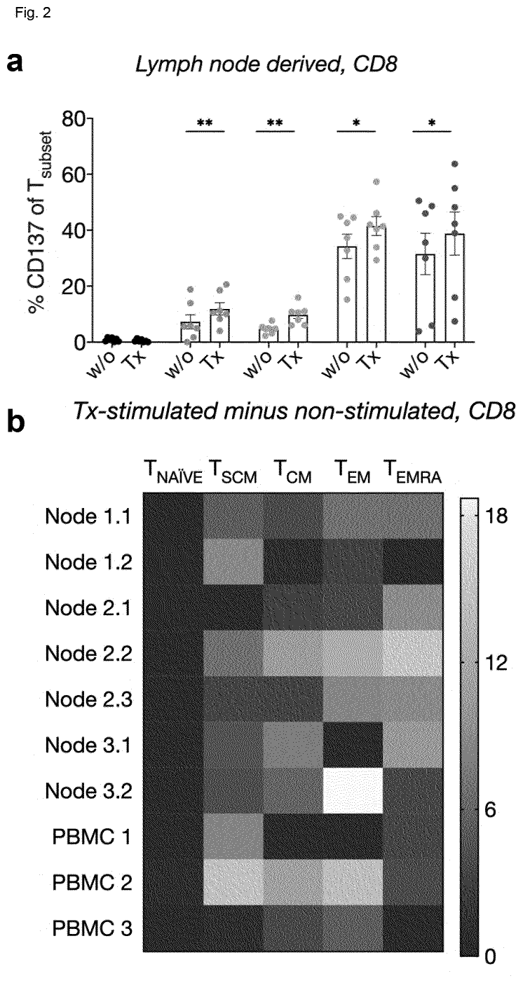

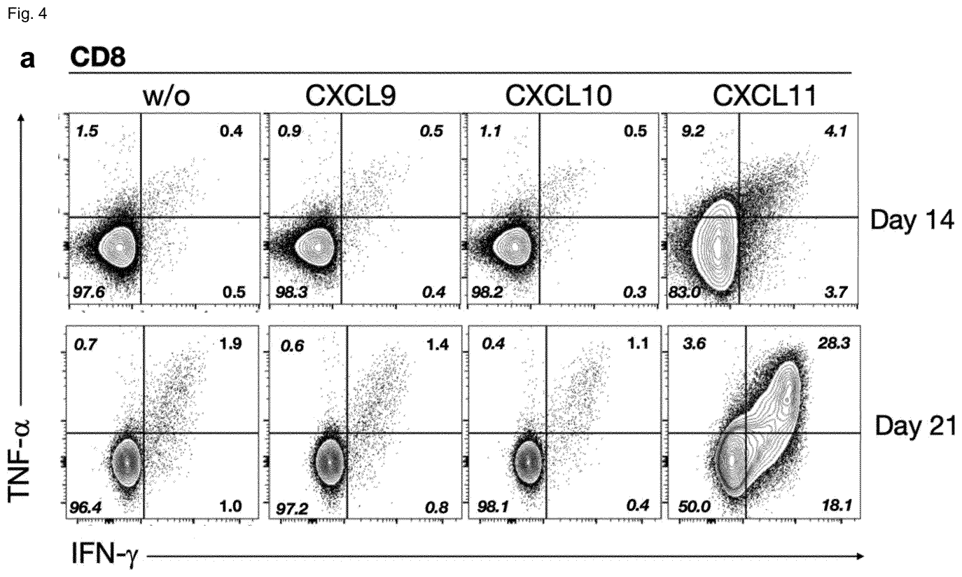

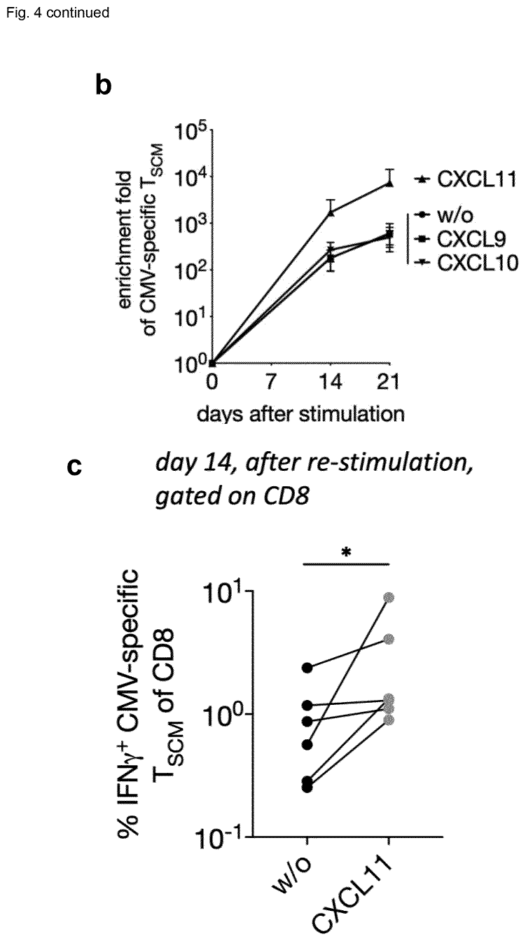

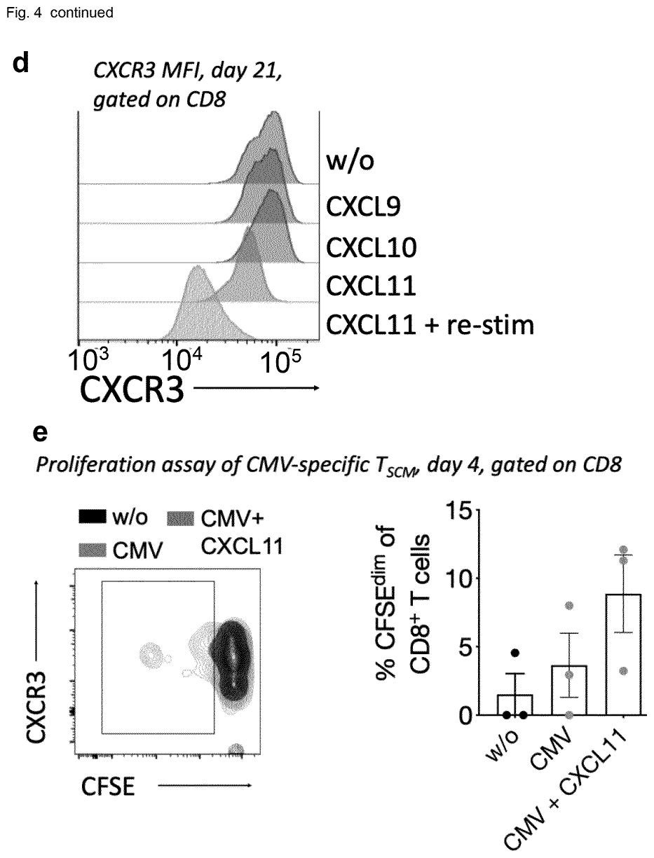

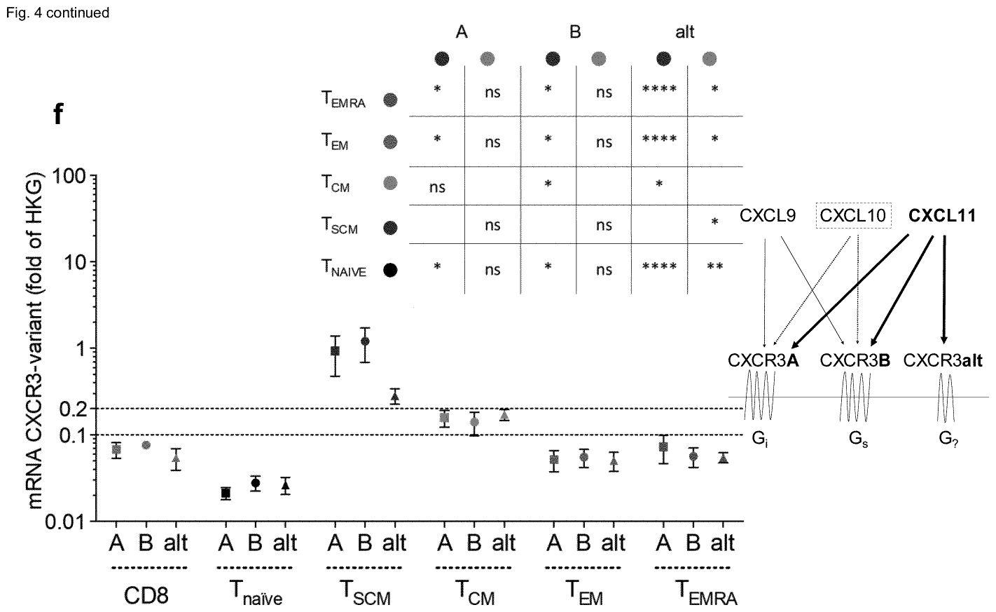

1×10⁶ million human PBMC were initially seeded in 200 μL RPMI, 10% FCS, 1% Penicillin/Streptomycin into the upper chamber of 24 transwell plates with 3 μm pore size (Corning) (FIG. 4a). 600 μL of CXCL9, CXCL10 or CXCL11 in media was added to the lower chamber and migration assays were conducted for 3 h. The chemotactic index (CI) describes the absolute number of migrated cells in the lower chamber with either CXCL9/10/11 normalized to the absolute number of migrated cells in the lower chamber without chemokine. To calculate absolute numbers of migrated subsets of CD8+ T cells, the fraction of each T cell subsets was defined by flow cytometry of the migrated cells according to phenotypic subset characterization (FIG. 2e). Frequencies of migrated CD8+ T cell subsets were assessed by flow cytometry.

T_{SCM}-Expansion Protocol

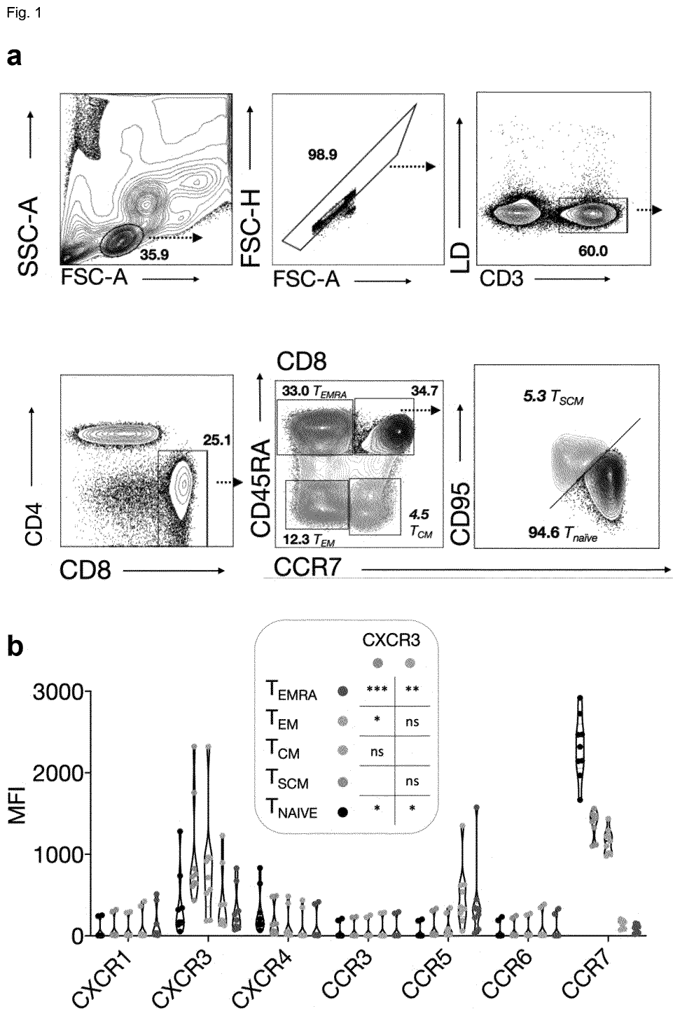

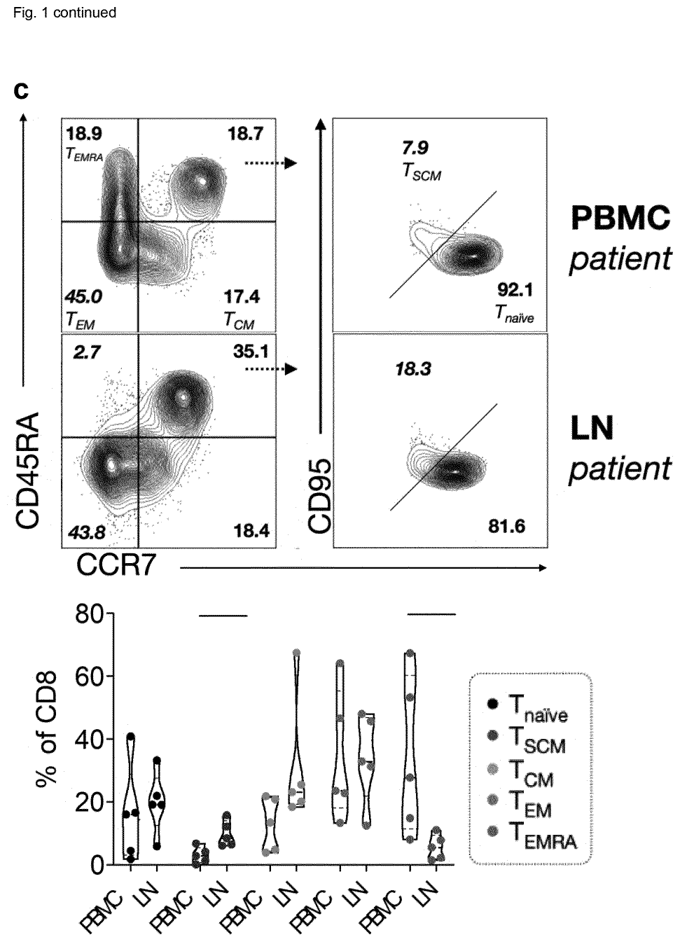

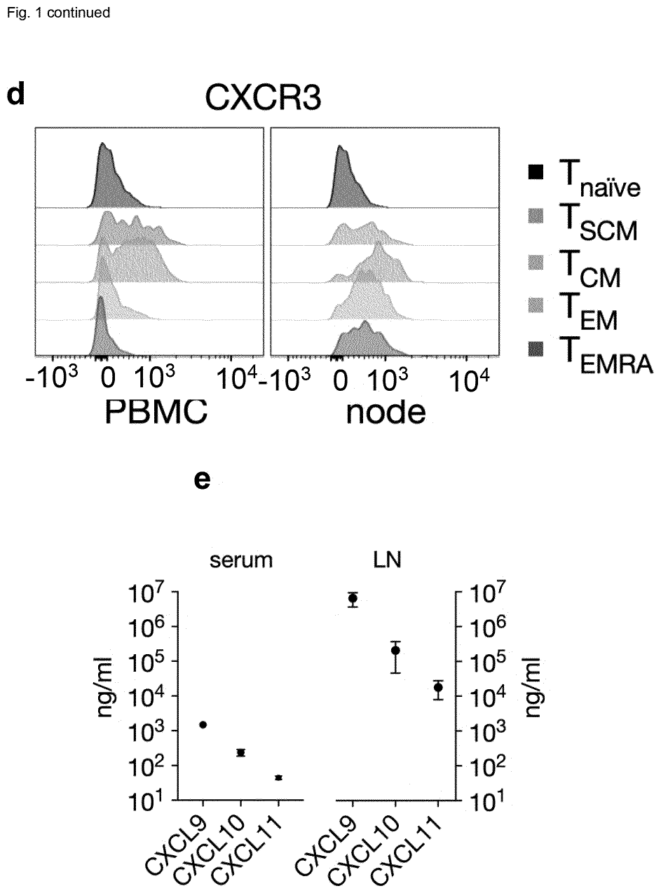

PBMCs were enriched via FACS for a CD3⁺CCR7⁺CD45RA⁺ T cell population on a BD FACS Aria II SORP (BD Bioscience) using the gating strategy in FIG. 1a. Sorted T cells were rested overnight, activated by irradiated (30 Gy) and CD3-depleted (MicroBeads; Miltenyi Biotech). autologous PBMCs were pulsed with CMV_{pp65/IE1} overlapping peptide-pool at a ratio of 10:1 (T cell:feeder). CMV_{pp65/IE1} peptide pools consisted of 15-mer peptides overlapped by 11 amino acids (JPT Peptide Technologies, Berlin, Germany) and were reconstituted in DMSO. After stimulation, cells were cultured in complete medium including recombinant human IL-7 and IL-15 each at 10 ng ml⁻¹ (CellGenix) at 37° C. and 5% CO2 in humidified incubators. At day 7, cultured cells were re-stimulated with freshly isolated, peptide-pool pulsed and irradiated CD3-depleted autologous PBMCs at a ratio of 10:1 (T cell:feeder). At day 14 and 21, expanded T cells were tested for antigen-specificity by their ability to recognise peptide-loaded target cell, measured by CD137 upregulation. Target cells were autologous lymphoblastoid B cell lines (LCLs) transformed with the B95-8 EBV strain and generated as previously described (Heslop, H. et al. Nat. Med. (1996) 2: 551-555). Re-stimulation for cytokine measurements was performed for 12 hrs, 11 hrs in the presence of 1 μg ml-1 brefeldin A (Sigma-Aldrich). The initial frequency of antigen-specific T_{SCM} within the CCR7⁺CD45RA⁺ T cell population was assessed via peptide-pool stimulation of freshly isolated PBMC. To measure proliferation, T_{SCM} were labelled with CFSE according to the manufacturer's instructions (Thermo-Fisher Scientific) and spiked into T_{NAIVE} at their initial frequency. Where indicated, cells were stimulated with CMVIE-1/pp66-overlapping peptide pool pulsed antigen-presenting cells. Proliferation for CD8⁺ T cell was assessed by the percent of CFSE diluted cells following 96-h culture in the presence or absence of CXCL11 and CMVIE-1/pp65-overlapping peptide pools.

Intra-Tumoural Cytokines Measurement

Cytokines were assessed in protein extracted. Luminex technology (Bio-Plex® 200 System, BioRad) was applied using multiplex assays (Merck) (Tab. 1). For each sample, the respective optical density values of the analyte concentration were assessed via a calibration curve and subtraction of the blank. The mean concentrations and standard deviations of the samples were calculated.

Intra-Tumoural Analysis of mRNA CXCR3-Variants

1 μg RNA from TURBT-specimens of the 46 BC-patients was used for cDNA synthesis according to the QuantiTect Reverse Transcription Kit manual (Qiagen). Quantitative real-time PCR (qRT-PCR) analysis was performed using TagMan PCR, containing FAM-BHQ1—labelled probes. mRNA CXCR3-variants were measured via TagMan qRT-PCR assays. To measure the main variant CXCR3A mRNA (NCBI reference sequence: NM 001504.1), the TagMan Universal PCR Master Mix was used with the probe Hs00171041_m1 (ABI) was used. To measure the CXCR3-splicing variants, two RT-qPCR panels specific for CXCR3B and CXCR3alt were designed (FIG. 1). The probe for CXCR3B (5′-TCACTATCCCAGAGCCCAG-3′) (SEQ ID NO 016), was designed specific for the extension site of CXCR3B and primers set as F: 5′-CCGTACTTCCTCAACTCCATCCGCT-3′ (SEQ ID NO 017) and R: 5′-TCCTATAACTGTCCCCGCC-3′ (SEQ ID NO 018) based on NCBI reference sequence NM 001142797.2. The probe for CXCR3alt (5′-CCGGAACTTGACCCCTGTGGGAAG-3′) (SEQ ID NO 019) was designed to hybridize to the CXCR3alt-specific sequence that arises from the joining bases due to post-transcriptional exon skipping (Ehlert, J. 2004), the primers for CXCR3alt were set as forward (F): 5′-CACGACGAGCGCCTCAA 3′ (SEQ ID No. 020) and reverse (R): 5′-GTTGGGGCAGCCCAGG-3′ (SEQ ID No. 021) based on NCBI reference sequence XM 005262257.3. For the design, Snapgene software 4.3.11 (GSL Biotech LLC) was utilised. Expression levels of target genes were measured in duplicate, using the ABI Prism 7500 Sequence detection system and associated software (all from ABI). To perform normalisation, mRNA of Hypoxanthine-guanine phosphoribo-syltransferase (HPRT) was used as house-keeping gene (HKG) and expression levels of target genes were calculated as fold of HKG. All expression levels were analysed in duplicate using the ABI Prism 7500 Sequence detection system and associated software (all from ABI). Tissue expression of target genes was normalized by the geometric mean of IP08 and CDN1B.

Statistical Analysis

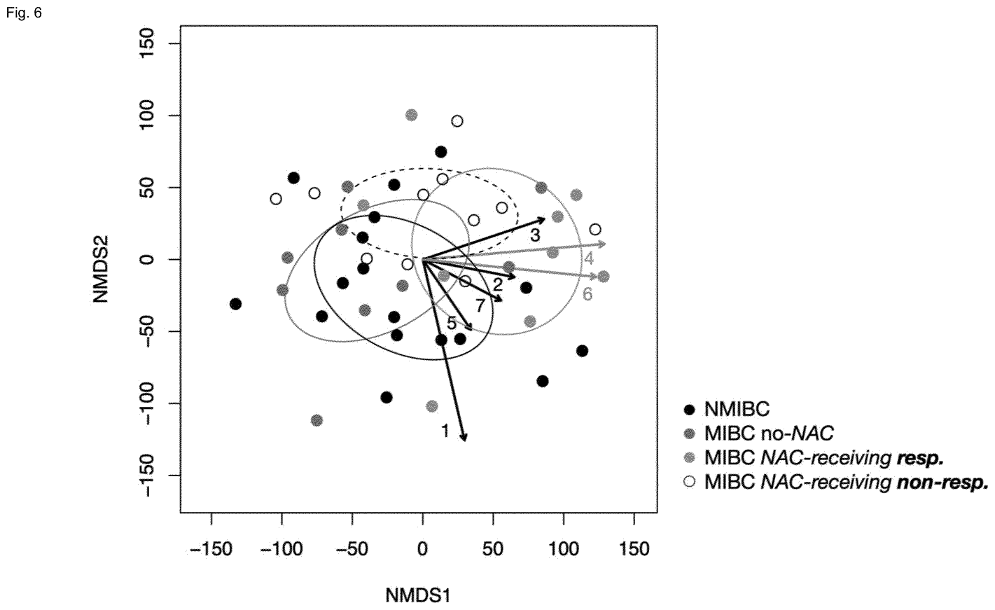

GraphPad Prism 8 (GraphPad Software) and R¹⁷ (version 3.5.2) were used to generate graphs and carry out the statistical analysis of data. To test for a normal Gaussian distribution, the Kolmogorov-Smirnov test was employed. RT-PCR data were log 2- and protein data were arsinh-transformed for display and prior to statistical analyses. Ct values below detection limit, i.e. above 40 (11%), were imputed using nondetects R package. In tumour samples, non-detects were not present, but 15 out of 90 measured protein analytes did not show sufficient expression, i.e. median absolute deviation above 0 across 46 tumour specimens, and were therefore excluded from statistical analyses. In serum samples, missing protein data (1.1%) were imputed using missForest R package. Cox proportional hazards models were fitted using coxph and cutp functions (survival package) to determine optimal split points to display Kaplan-Meyer curves of dichotomized data. Individual patients' restricted mean survival times as shown in FIG. 6b are given by the areas under the survival curve from origin to each event's timepoint. Hierarchical clustering of patient cytokine data was performed within patient subgroups (NMIBC, NAC-receiving MIBC, no-NAC MIBC) separately, using Euclidean distances between rank-transformed data and complete-linkage method. Global differences between patient subgroups in chemokine milieus were tested by permutational analysis of variance and visualized by nonmetric multidimensional scaling (vegan R package). Clustering of markers was conducted (with Ward's method) on a shrunken partial rank-correlation matrix across all 46 BC-patients using ConsensusClusterPlus package with a 1000-fold resampling scheme in order to identify co-regulated protein functional modules. Markers identified within the CD3-connected module were selected to obtain a sparse mixed Gaussian graphical model where response on NAC was included as a binary variable. To this end, a semiparametric correlation matrix from observed mixed-type data (14 continuous variables and response) of 20 NAC-receiving patients was estimated based on the latent Gaussian copula model, subsequently a sparse partial correlation matrix was selected using the graphical lasso with the extended Bayesian information criterion and presented with the qgraph package. Firth's bias-reduction method (implemented in brglm R package) was used to fit a logistic regression model with CXCR3alt and CXCL11 as predictors. Public single-cell RNA-sequencing (scRNAseq) data were obtained from NCBI's Gene Expression Omnibus (GEO). The scRNAseq data of two primary bladder cancer samples (MIBC), GEO Series accession number GSE130001, and of three primary healthy bladder samples (BH), GEO Series accession number GSE129845, were downloaded as UMI count matrices generated by the CellRanger pipeline (10× Genomics). Count data were normalized, low-quality cells and low-abundance genes removed, and subsequently, 17520 cells (BH: 337, 3527, 9576; MIBC: 3486, 594) clustered as well as t-SNE dimensionality reduction performed. The melanoma dataset, GEO Series accession number GSE72056 (GSE72056_melanoma_single_cell_revised_v2.txt) was log-normalized single-cell expression profiles of 19 tumour specimens along with the cell-type characteristic. In order to visualize expression of selected genes across different cell-types, each tumour was down-sampled to a maximum of 100 cells per cell-type to a total input of 3187 cells and subjected without further filtering to the t-SNE algorithm (utilizing Rtsne package with default hyperparameters). Bladder cancer gene-expression data as prepared by The Cancer Genome Atlas (TCGA, https://portal.gdc.cancer.gov) and preprocessed to RSEM normalized gene expression values by the firehose pipeline (https://gdac.broadinstitute.org) were downloaded using the curatedTCGAData R package.

### Example 1: Predictive Biomarkers for NAC Response in BC

To unveil the functional relevance of the treatment-naïve CXCR3-chemokine system associated with human anti-tumour immunity, primary tumour biopsies, routinely taken prior to the onset of platinum-based NAC, were collected from BC-patients that were categorised as either NMIBC or MIBC. A comprehensive retrospective characterisation of intra-tumoural cytokines and CXCR3-isoform expression in relation to anti-tumour responses induced by NAC was then performed.

CXCR3 is Highly Expressed on Early-Differentiated Peripheral CD8⁺ T Cells and Enriched in CXCL9/10/11 high Lymph Nodes of MIBC-Patients.

To investigate the heterogeneous chemokine receptor expression on CD8⁺ T cells, CXCR3 expression was compared with CXCR1, CXCR4, CCR3, CCR5, CCR6, and CCR7 on discreet CD8⁺ T cell functional subsets from human healthy donors (FIG. 1a). High CXCR3/CCR7 expression characterised early-differentiated CD8⁺ stem cell memory (T_{SCM}) and central-memory (T_{CM}) T cells, whereas on CD8⁺ naïve (T_{N}), late differentiated CD8⁺ effector-memory (TEM) and CD8⁺ terminally differentiated effector-memory T (T_{EMRA}) cells CXCR3 expression was low (FIG. 1b). CXCR3 promotes homing of CD8⁺ T cells to the secondary lymphoid organ and their prepositioning within the lymph node. Therefore, tumour-adjacent lymph nodes are an important reservoir for early-differentiated tumour-reactive CD8⁺CXCR3⁺ T cells. The tumour-adjacent lymph nodes of five MIBC patients undergoing RC were analysed for T cell subset distribution, T cell mediated anti-tumour reactivity, and CXCR3-receptor/ligand expression within the context of a tumour neighbouring microenvironment. Higher frequencies of T_{SCM}-cells and T_{CM}-cells were observed within the lymph nodes compared to the peripheral blood of MIBC patients (FIG. 1c) and higher CXCR3 expression was detected on nodal CD8⁺ T cells subsets (FIG. 1d). CXCR3-ligands CXCL9/10/11 were higher in the lymphatic tissue of patients compared to serum levels (FIG. 1e), reflecting a chemokine gradient which promotes LN homing of early-differentiated CXCR3⁺ T cells.

Indeed, when LN-derived cells were stimulated for 12 h with autologous bladder tumour lysates, antigen specific activation measured by increased upregulation of CD137 could be observed in memory and effector CD8+ population compared to the naïve compartment, suggesting an enrichment of tumour specific T cells (FIG. 2).

High CXCR3-Isoform Expression on Early-Differentiated CD8⁺ T Cells Associates with Differential Functional Outcome Mediated by the CXCR3-Ligand Family

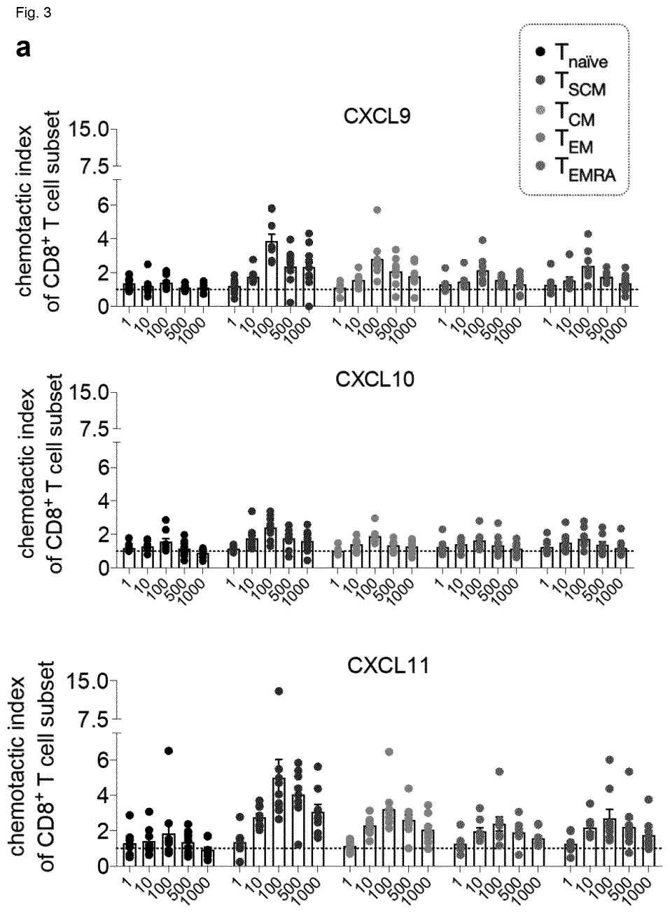

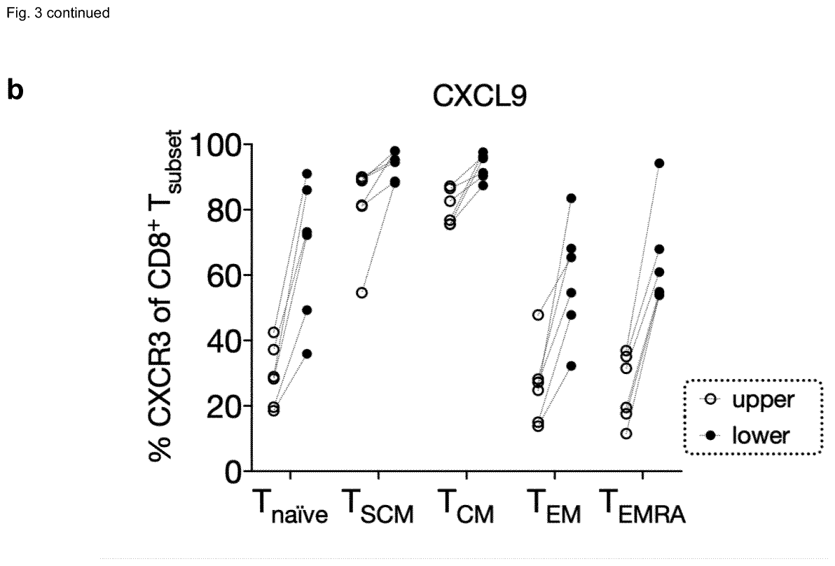

In-vitro migration assays (FIG. 3a) found that CXCL9, CXCL10, and CXCL11 induced chemotaxis of CXCR3highCD8⁺ TSCM and TCM (FIG. 3b). Maximum cell migration of CD8⁺ T_{SCM} was observed at 100 ng/ml of any CXCL9/10/11. This implies all CD8⁺ T cell subsets, and particularly CXCR3+ stem cell memory cells, are endowed with an enhanced responsiveness to specific CXCR3 ligands. To determine the effect of CXCL11 on CD8⁺ T cell subsets, an antigen-specific in vitro expansion culture was utilised to examine the functional outcome for CXCR3-ligation on early-differentiated CD8⁺ T_{SCM}-cells important for mediating antitumour responses. CXCL11 but not CXCL9/10 amplified the enrichment of antigen-specific CD8⁺ T_{SCM}-cells concomitant with the downregulation of CXCR3 (FIG. 4a-d). Furthermore, CXCL11 accelerated in-vitro proliferation of CD8⁺ TSCM in short-term cell division assays (FIG. 4e). As in vitro applied CXCL11 mediates an activating effect on virus-specific CXCR3^{high}CD8⁺ T cell, tumour expression of CXCL11 may amplify cancer-directed responses of early-differentiated T cells. The alternative spliced transcript CXCR3alt has been reported to exclusively bind CXCL11 (Ehlert, J. 2004) and elicit downstream signalling upon CXCL11-ligation (Berchiche, Y. A. and Sakmar T. P. (2016) 90: 483-495). To determine whether the CXCR3-isoforms expressed by different CD8⁺ T cell subsets have a functional significance in BC, a RT-qPCR-panel for the CXCR3A/B/alt-variants was employed to measure variant expression in patient samples. The highest transcriptomic activity of all three CXCR3-variants was found in peripheral T_{SCM}-cells and T_{CM}-cells, compared to CD8⁺ T cell subsets. The highest expression of the alternative spliced transcript CXCR3alt was detected in T_{SCM}-cells. Further, CXCR3alt was the most-differentially expressed CXCR3 transcript within the T cell subsets (FIG. 4f). High expression of CXCR3alt therefore identifies T_{SCM}-cells predisposed to be functionally responsive to CXCL11-ligation in the tumour microenvironment.

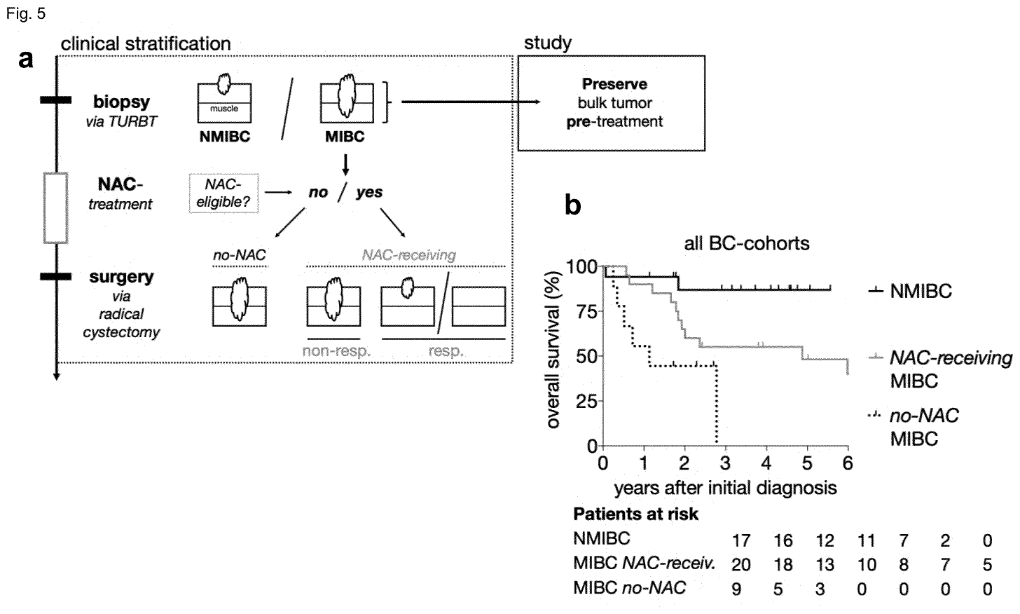

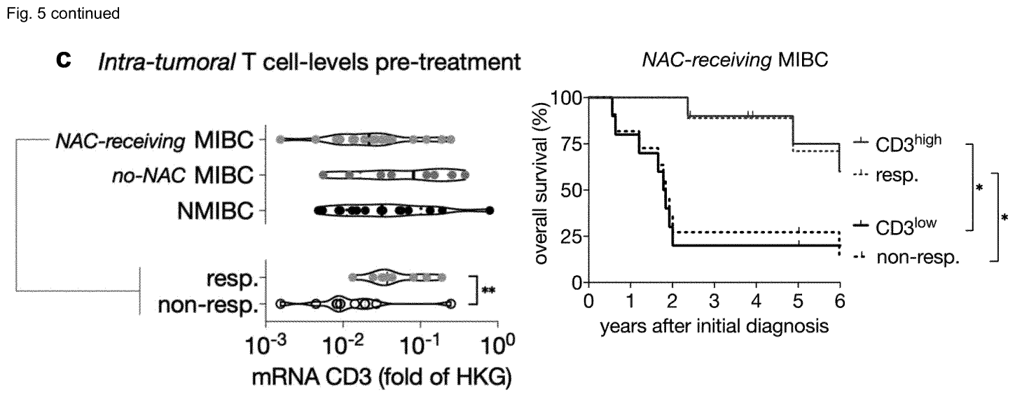

CXCL11 is Associated with Intra-Tumoural T Cell Infiltration Marks NAC-Responsive Patients A cohort of 46 BC-patients was used to dissect the putative roles of the CXCR3-chemokine system in anti-tumour responses induced by chemotherapy (FIG. 5a). Patients were assigned as either NMIBC (17/46) or MIBC (29/46) via primary endoscopic biopsy; i.e. TURBT (transurethral resection of the bladder tumour). In the follow-up, 20/29 MIBC-patients were clinically fit and thus eligible to receive platinum-based NAC before RC. Responders to NAC were identified due to pathoanatomical downstaging of the tumour histology in the RC. The response to NAC was also a surrogate marker of long-term survival. In this cohort, 9/29 MIBC-patients were clinically unfit to receive NAC (no-NAC) due to age/co-morbidity/impaired renal function. In the patient cohort, a good OS of NMIBC, intermediate OS of NAC-receiving MIBC, and poor OS of no-NAC MIBC patients was observed (FIG. 5b). To examine whether these prognostic differences were associated with overall levels of tumour-infiltrating T cells, pre-treatment CD3 mRNA expression levels were assessed as a surrogate marker for T cell infiltration, reflecting capacity for protective antitumour immunity. Comparable levels of intra-tumoural T cell infiltration were detected in the three BC-subgroups. Within NAC-receiving MIBC patients significantly higher intra-tumoural T cell levels were found in responders compared to non-responders (FIG. 5c).

The formation of functional intra-tumoural T cell structures requires effective chemotactic homing within a favourable inflammatory milieu. However, it remains unknown whether the CXCR3-ligands, CXCL9/10/11, are part of the BC-specific cytokine signature or whether the distinct CXCR3-ligands are associated with the anti-tumour response. A multiplex-based detection of pre-treatment cytokines was performed on lysate from primary BC-biopsies. In NAC-receiving MIBC, the tumours were assigned to high versus low inflamed states characterised by cytokines and chemokines in two distinct clusters (4 & 6) that segregated the NAC-responding MIBC-patients from the remaining BC-subgroups in a multidimensional scaling model (non-responding MIBC, no-NAC MIBC, NMIBC) (FIG. 6). Cluster 4 contained the CXCR3-ligands CXCL9/10/11 including IFN-gamma, CCL2/3/4/19, CXCL12/13 and IL-16. Further, a NMIBC-associated signature (cluster 1: IFN-beta, IL-28beta, IFN-alpha-2, IL-13, IL-29, IL-34, IL-19, IL-11, XCL1, IL-3) separated from the MIBC-milieu. In no-NAC MIBC, a reduced inflammatory signature was evident. Correlation analysis was used to isolate single markers associated significantly with T cell infiltration and with the response to NAC, respectively. Cluster 4 including the CXCR3-ligands exhibited the strongest correlation with T cell levels throughout all BC-subgroups. Overall, 24 cytokines significantly correlated with T cell infiltration, and 9 cytokines significantly correlated with the response on NAC, with the CXCR3-receptor ligand CXCL11 exhibiting the highest significance level of all markers (p<0.001), suggesting that CXCL11 might serve as a potent marker to predict the response to NAC.

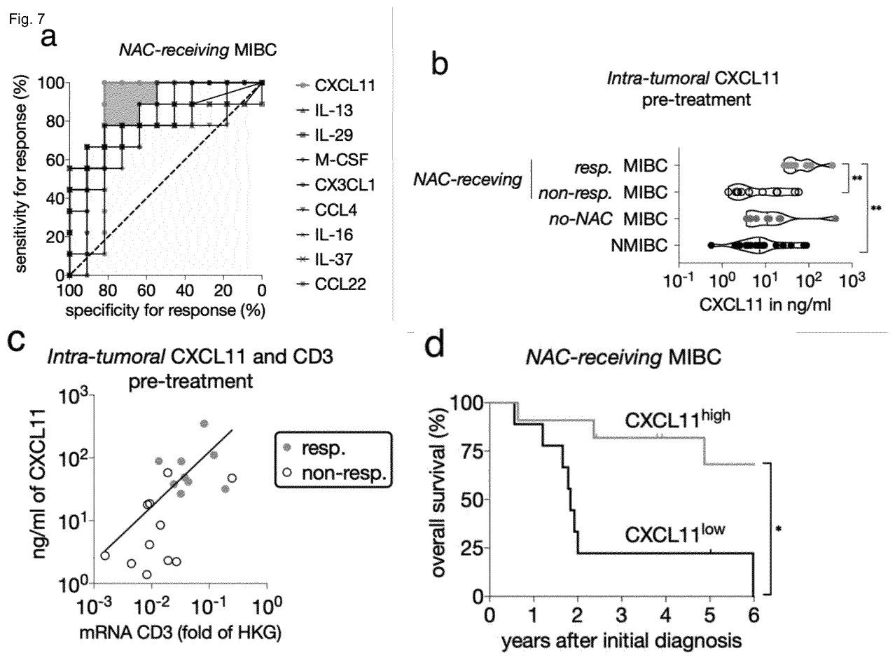

Receiver operating characteristic (ROC)-curves were generated to analyse the diagnostic ability of all significantly different cytokines to predict the response to NAC. CXCL11 was the most sensitive marker for predicting the response to NAC (FIG. 7a). NAC-responding MIBC-patients exhibited significantly higher intra-tumoural concentrations of CXCL11 than non-responding MIBC-patients and NMIBC-patients (FIG. 7b). Analysing the serum levels of cytokines pre-treatment, CXCL11 was not significantly elevated in NAC-responding patients. Furthermore, there was a positive correlation of intra-tumoural CXCL11 levels with the levels of tumour infiltrating T cells (FIG. 7c) and improved OS of CXCL11 high compared to CXCL11″ tumours in NAC-receiving MIBC (FIG. 7d). Together, these data indicate that intra-tumoural CXCL11 is a critical component of the immune response which mediates beneficial effects of NAC, and CXCL11 is a biomarker which accurately identifies NAC-responder patients before they receive NAC treatment.

CXCL11 and CXCR3alt as Dual Stratification to Predict the Response to NAC in MIBC

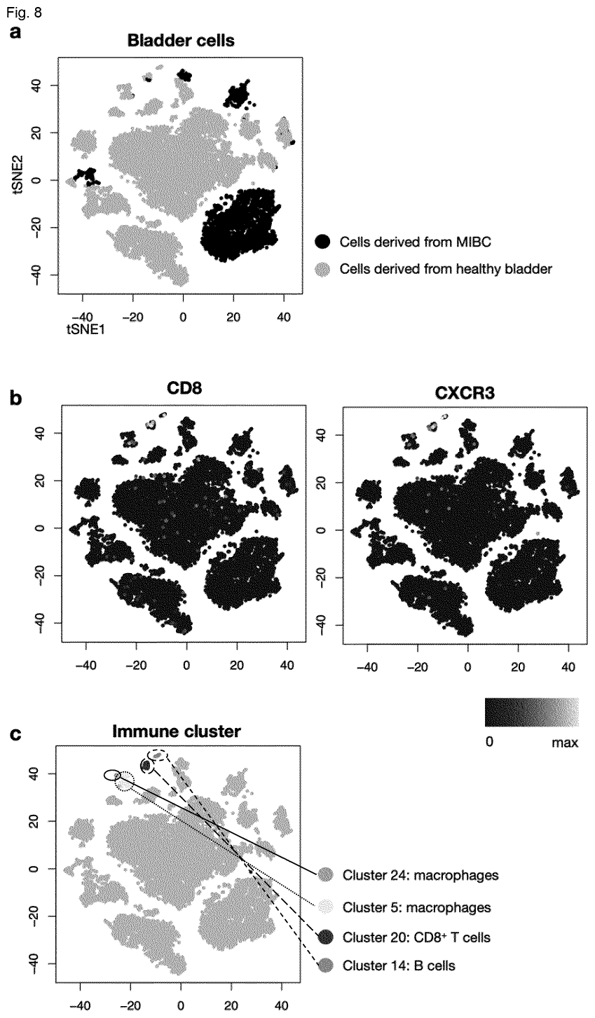

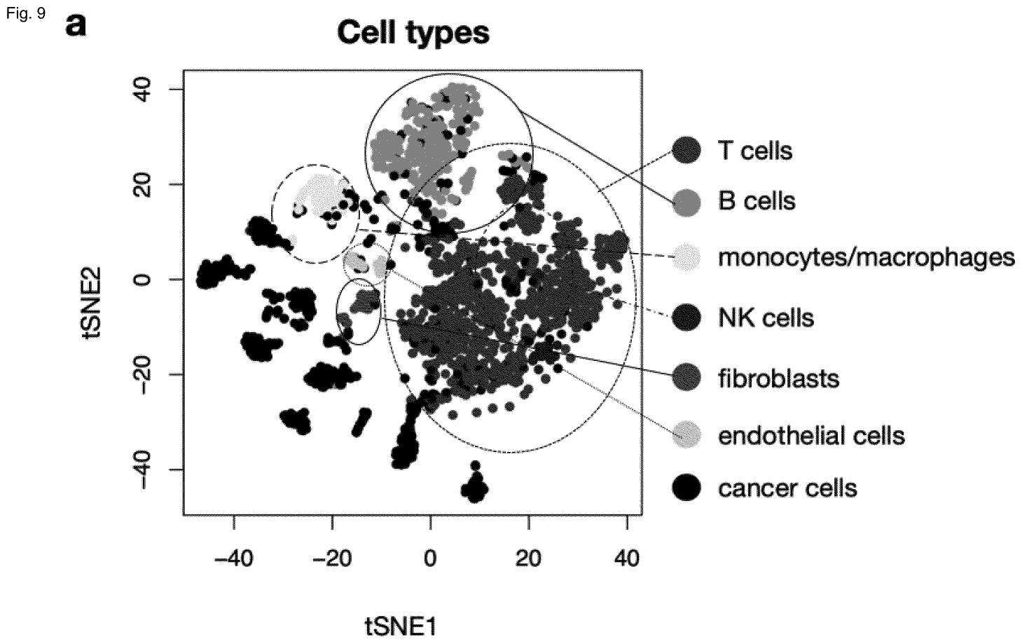

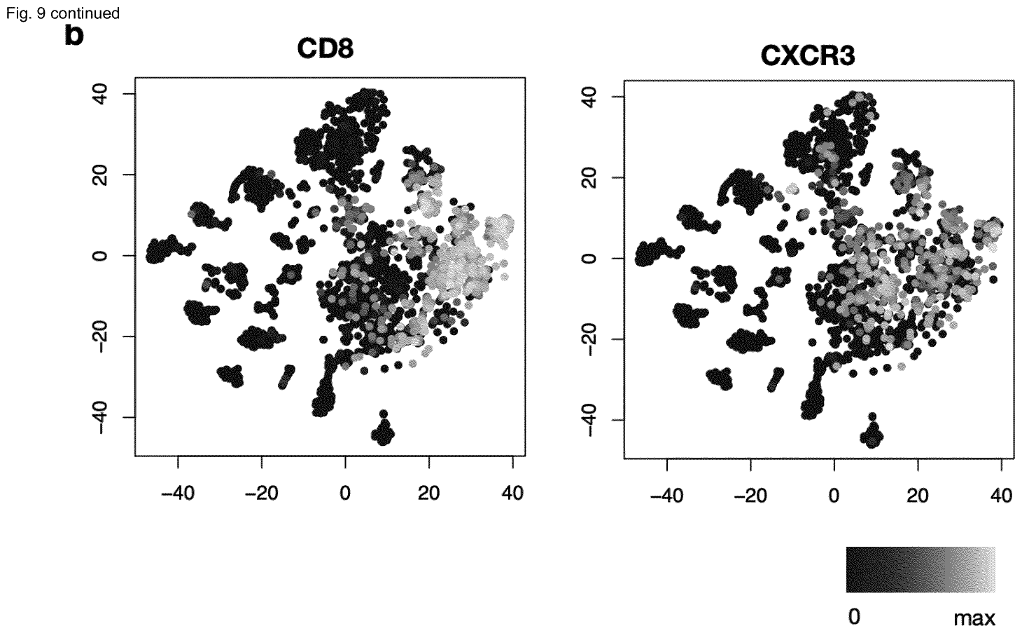

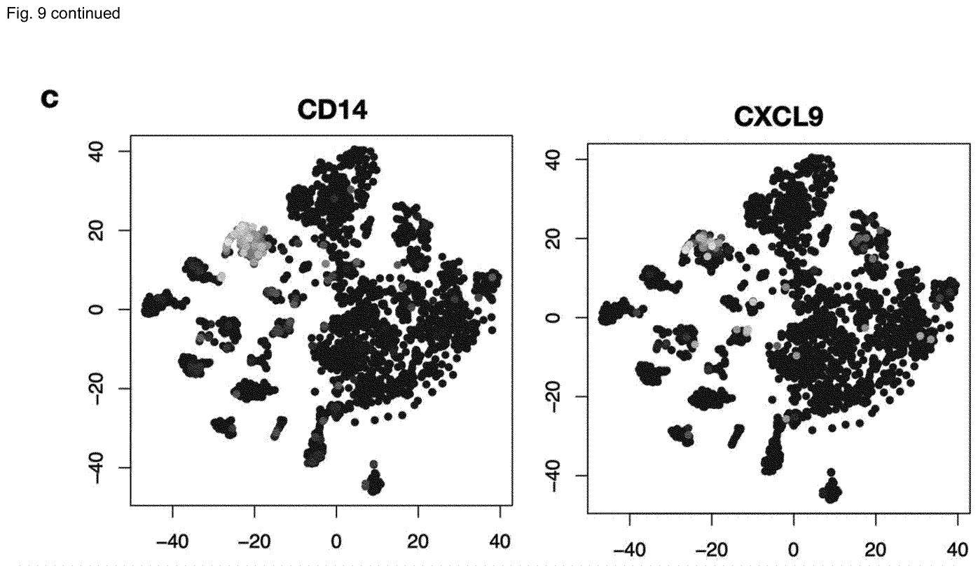

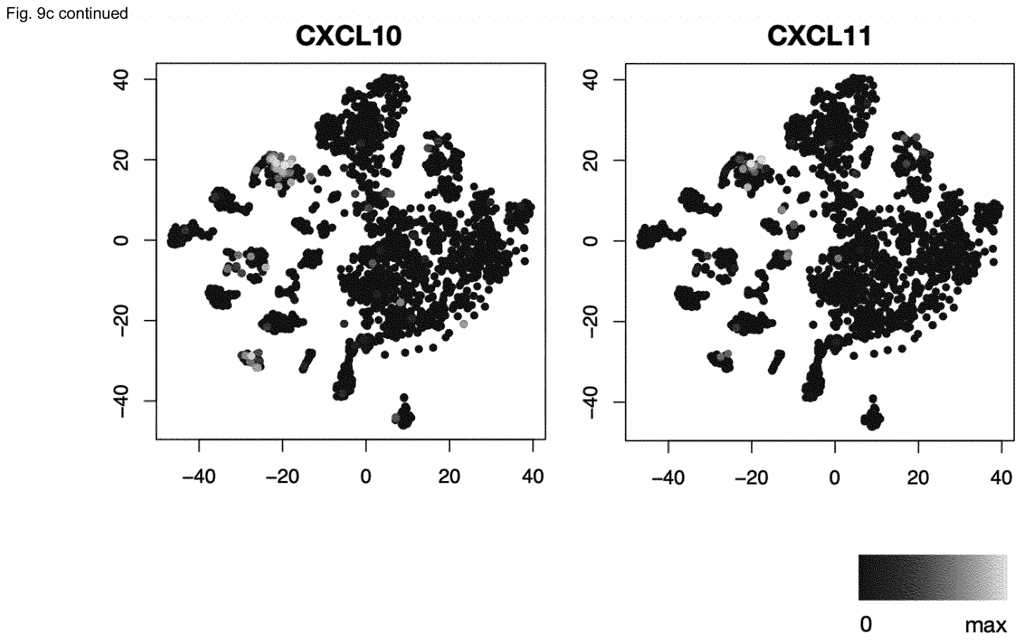

To dissect which cells in the healthy bladder and the bladder tumour express CXCR3, CXCR3-expression was measured in human bladder by accessing publicly available single-cell RNA-sequencing data of three healthy bladder homogenates and two cancer cell-enriched MIBC-specimens (see data availability). This data indicated an absence of CXCR3 expression in healthy bladder cells as well as in cancer cells, whereas CXCR3 was expressed in tissue-infiltrating T cells (FIG. 8). In human melanoma cancer dataset, CXCR3 expression was also limited to CD8+ tumour infiltrating T cells in melanoma in previously published single-cell RNA-sequencing data (FIG. 9). In BC and melanoma, CXCR3-expresssion is restricted to tumour-infiltrating immune cells that predominantly comprise T cells, although other cancer types may employ CXCR3-expression for tumour progression and metastasis (Billottet C. (2013) 1836: 287-295).

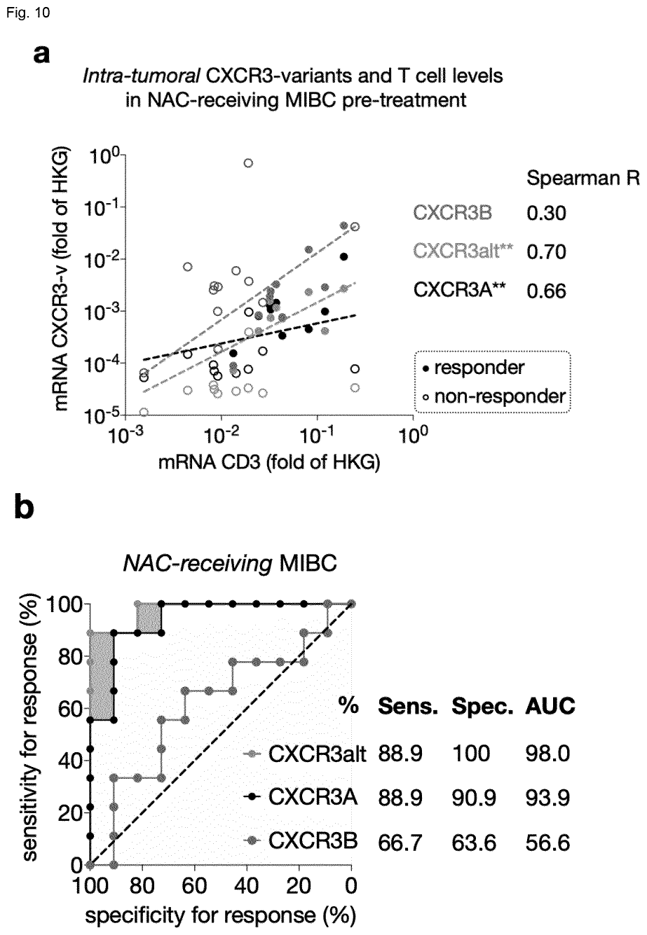

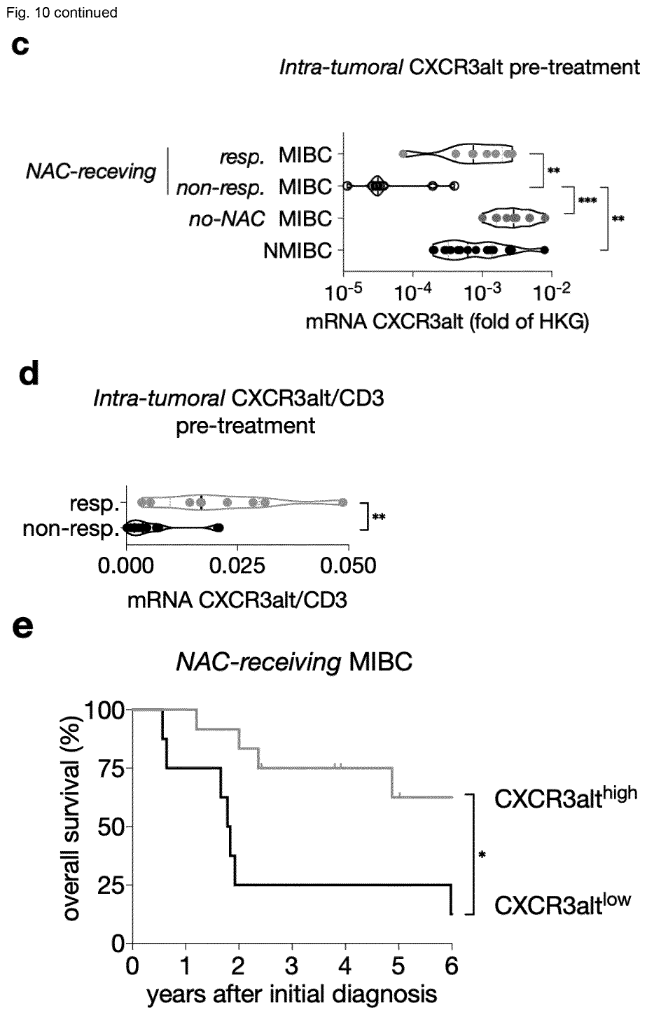

The alternative spliced transcript CXCR3alt has been reported to exclusively bind CXCL11 (Ehlert, J. 2004) and elicit downstream signalling upon CXCL11-ligation (Berchiche, Y. A. and Sakmar T. P. (2016) 90: 483-495). To determine whether the CXCR3-isoforms expressed by different CD8⁺ T cell subsets have a functional significance in BC, a RT-qPCR-panel for the CXCR3A/B/alt-variants was employed to measure variant expression in patient samples. In the BC-cohort, intra-tumoural mRNA expression levels of the CXCR3-isoforms (CXCR3A/B/alt) were tested for correlation with T cell levels. The mRNA-expression levels of CXCR3A/alt, but not CXCR3B, significantly correlated with the T cell levels in NAC-receiving MIBC (FIG. 10a). Just as for CXCL11 concentration, CXCR3-isoform expression can also predict patient response to NAC. The mRNA-expression of CXCR3A, and more stringently, CXCR3alt, accurately predicted the response to NAC (FIG. 10b). Of note, non-responding MIBC-patients exhibited a significantly lower mRNA-expression of CXCR3alt compared to all other BC-subgroups, including responding MIBC-patients (FIG. 10c), which was also confirmed after an additional normalisation to T cell-levels (FIG. 10d). The clinical significance of CXCR3alt in the response to NAC was confirmed by a strong association with OS in MIBC-patients (FIG. 10e).

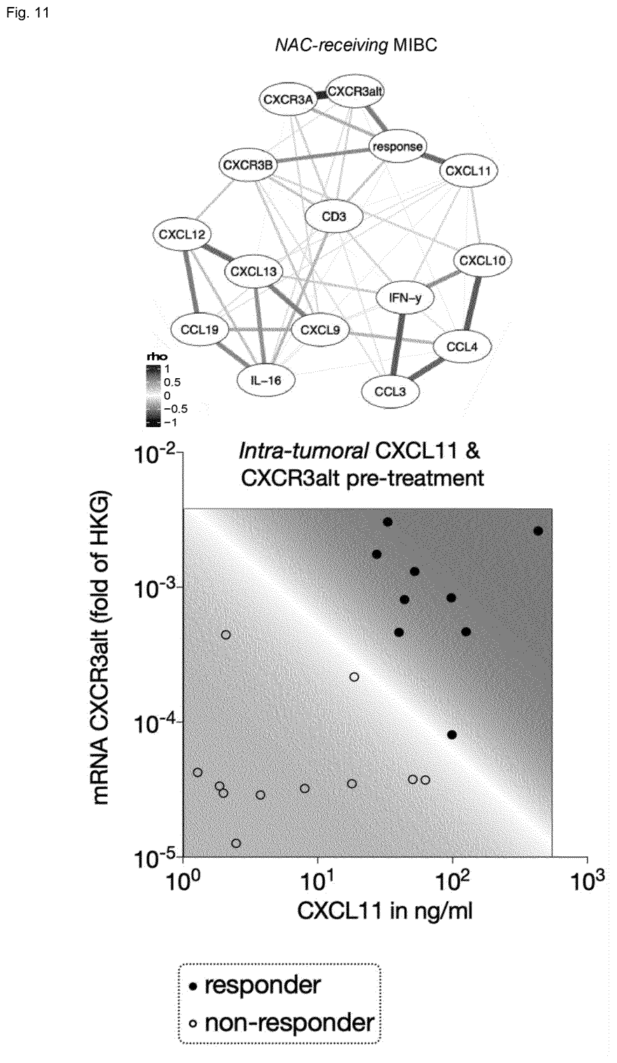

To scrutinize the dependencies between the CXCR3-chemokine system and the inflammatory tumour milieu, pairwise correlation analysis was used to detect intra-tumoural co-regulation between the CXCR3-isoforms, T cell levels and cytokine expression, including the mRNA of the CXCR3-isoforms, the mRNA of CD3 and the cytokine protein levels. Using a robust clustering technique, the three CXCR3-isoforms and CD3 grouped with the three CXCR3-ligands (CXCL9/10/11), and IFN-gamma, CCL3, CCL4, IL-16, CCL19, CXCL12, CXCL13 in one specific cluster (FIG. 11, upper panel). To dissect functional dependencies and to estimate the capacity for the anti-tumour response in this cluster, a network analysis was used, with the clinical response to NAC as a binary variable. CD3 was identified as central node within the network, an IFN-gamma module (IFN-gamma, CXCL10, CCL3, CCL4), a lymphoid-like module (IL-16, CCL19, CXCL12, CXCL13, CXCL9), strong co-regulation between CXCR3A and CXCR3alt, and lastly CXCR3B as a negative factor. Notably, a direct relation was found between CXCL11 and the response to NAC and independently, between CXCR3 variant expression and the response on NAC (FIG. 11, Tab. 2).

Expression levels thresholds and the 95% confidence interval (CI) for CXCL11 protein, or the CXCR3 isoforms measured by quantitative PCR which predict a positive outcome to NA treatment were as follows:
CXCL11 is more than 22.4 pg per 10 mg of tissue, CI: [13.98, 35.44]
CXCR3A is more than 2^{(-11.97)} times that of the HKG. CI: [2^{(-12.3)}, 2^{(-11.0)}]
CXCR3alt is more than 2^{(-11.27)} times that of the HKG. CI: [2^{(-13.8)}, 2^{(-10.4)}]
CXCR3B is more than 2^{(-8.43)} times that of the HKG. CI: [2^{(-11 .9)}, 2^{(-4.9)}]

Applying CXCR3alt-CXCL11 as a dual marker stratification for NAC-receiving MIBC patients using a logistic regression model, responding and non-responding MIBC patients could be completely separated prior to NAC treatment (FIG. 11 lower panel, Tab. 3). Taken together, the assessment of CXCL11-protein levels and CXCR3alt mRNA-expression in BC-biopsies from MIBC patients enables for the prediction of the response to NAC.

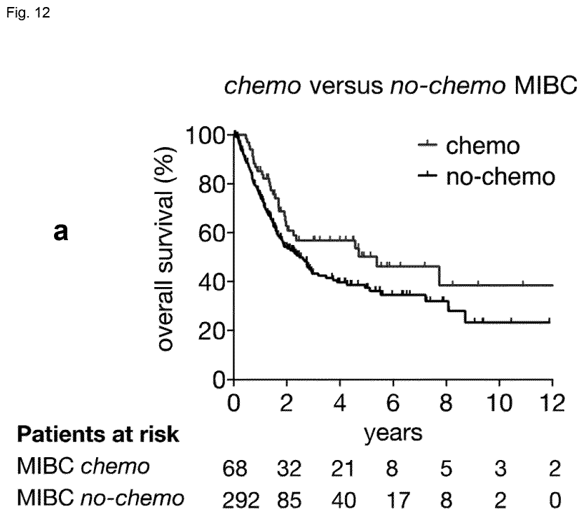

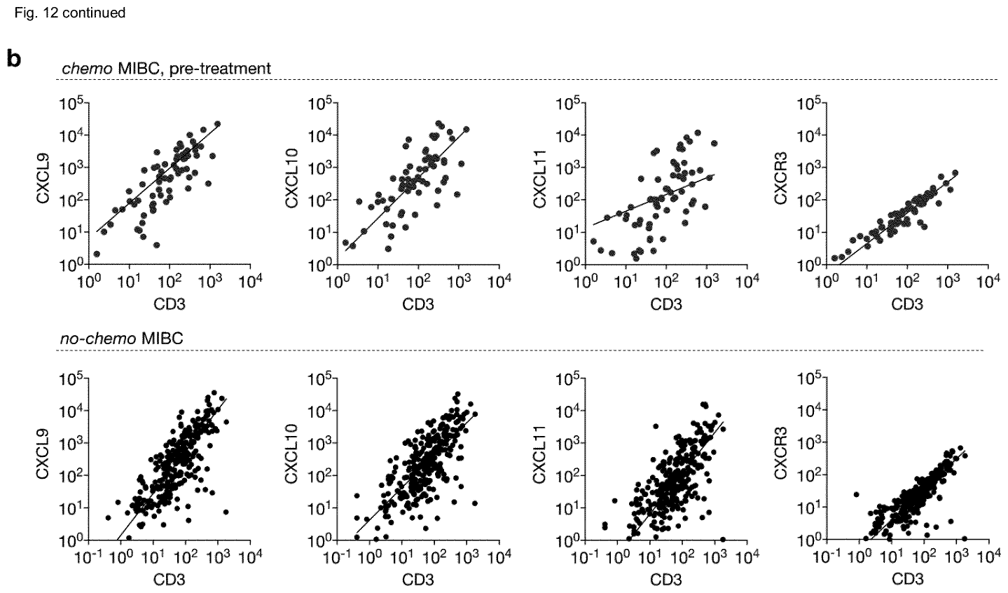

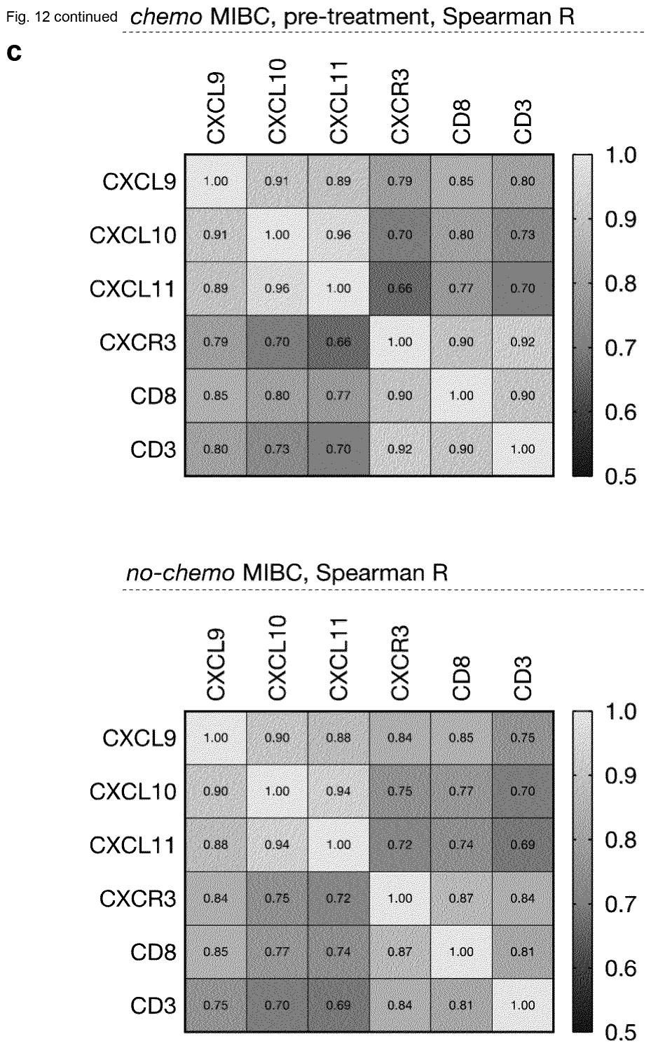

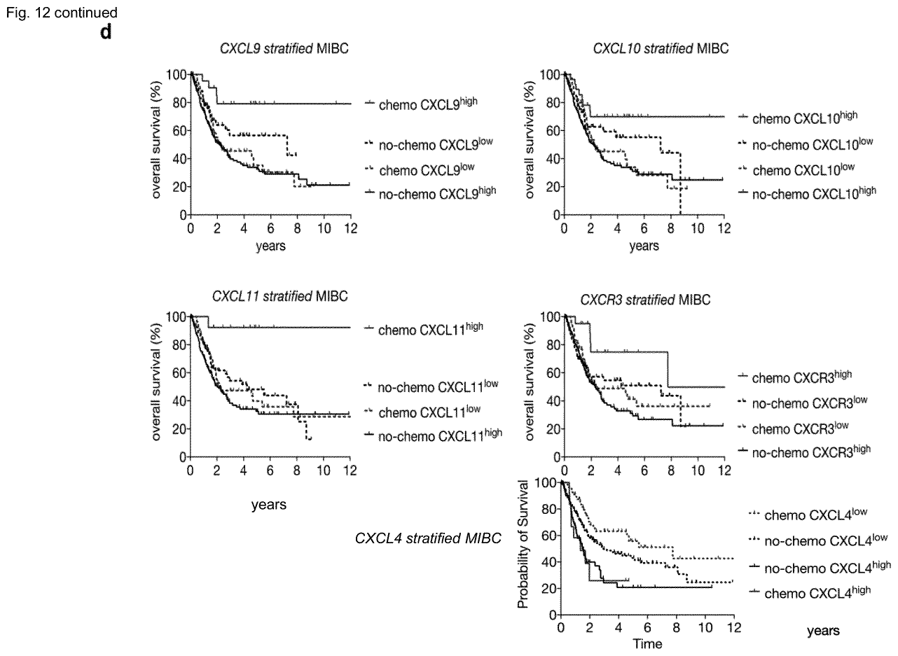

For external validation, the pre-treatment mRNA expression levels were analysed in tumour specimens of an independent MIBC patient cohort provided by the TCGA (The Cancer Genome Atlas: a cohort of 68 chemotherapy-receiving MIBC patients to 292 chemo-naïve MIBC patients). In this cohort, chemotherapy treatment was associated with slightly improved OS (FIG. 12a). In all treatment-naïve samples, there was a significant positive correlation of intra-tumoural CD3 and CXCL9/10/11 mRNA expression level, confirming the expression of this chemokine system correlates with the level of T cell activity present in the tumour (FIG. 12 b and c). CXCL11 mRNA high tumours were prognostic for improved OS in the chemotherapy-receiving MIBC cohort, but not the chemo-naïve MIBC patient cohort, as were the CXCL9/10 mRNA expression levels in this large cohort, confirming that the amount of T cell activity as measured by the level of pre-treatment CXCR3 ligands is an effective biomarker linked to the outcome of neoadjuvant therapy (FIG. 12d, Tab. 4). This larger cohort additionally identified CXCL9 and CXCL10 as effective positive predictors. Although not identified in the T cell response cluster of chemokine protein analysis, the final member of the CXCR3 cytokine family, CXCL4 was also tested in the TCGA cohort, and was shown to be a negative predictor of neoadjuvant response in BC patients. The TCGA data comprise intra-tumoural mRNA gene expression levels in contrast to protein levels and thus does not allow for discrimination of the CXCR3alt isoform. Bulk CXCR3high mRNA tumours indicated improved OS compared to CXCR3low mRNA tumours (FIG. 12d). Total CXCR3high mRNA tumours poorly predicted improved OS compared to discrimination provided by each variant in the Swedish cohort (Tab. 4). The validation cohort confirmed the robust prognostic value of the CXCR3 chemokine family in a second NAC-receiving MIBC cohort, with different sample measurement protocols. The modest predictive power of bulk CXCR3 measurements suggests that the CXCR3 variants are superior biomarkers.

### Example 2: BC Patient Classification Using CXCL11 and CXCR3A or CXCR3alt Expression

Statistical models to predict the clinical outcome of the NIMBC patients to neoadjuvant therapy were developed based on the expression levels of the biomarkers, CXCL11 and CXCR3 splice variants in pre-treatment tissue samples. The predictive performance of biomarkers was assessed for either individual marker thresholds (Tab. 2), or predictive logistic regression models using two or more biomarker values (Tab. 3). The performance of each model in terms of predicting outcome to MVAC therapy was assessed by the AUC of ROC curves, and both the AIK and Brier model fitting scores.

The presence of CXCR3 in the cancer tissue samples was measured by real time quantitative PCR, using Taqman probes, providing a CT value. The CT value, or threshold cycle, is the cycle number at which the fluorescent signal of the reaction crosses a user-defined threshold, i.e. exceeds background level. The CT value is inversely related to the starting amount of target DNA. The Δ CT value is the difference in expression (CT) between the target gene and the CT of a control gene, a stable expressed housekeeping gene. Here the control CT is the arithmetic mean of two house-keeping genes, IPO8 and CDKN1B identified by the genorm algorithm. In the context of the present examples, the value for the biomarker is given by:

CXCR3alt=ΔCT CXCR3alt=CT(CXCR3alt)−((CT(IPO8)+CT(CDKN1B))/2)

CXCL11 was measured by a multiplexed cytokine bead array system, giving a concentration in pg per 10 mg of tumour sample. This value was then normalized to stabilize the variance of multiple measured proteins of different intensity measured by the multiplex system using a quasilogarithmic transformation described by:

CXCL11=arsinh(CXCL11 concentration in pg per 10 mg of tumour sample)=ln(x+(x2+1)^{0.5}).

The probability of responding to NAC is calculated by the formula:

p=1/(1+exp(−y)), where
y is a linear combination of the two explanatory variables.

The linear combination can be calculated including estimates for an intercept a and two regression factors β1, β2 for the variables:

y=α+β1(CXCL11)+β2(CXCR3alt)

Estimates of the regression coefficients obtained by maximum likelihood estimation with Firth's bias reduction method for the logistic regression model generated in example 1:

α=6.045

β1=1.303

β2=0.904

Probability of a patient responding favourably to NAC using levels of the two markers where unnormalized CXCL11=2.77 pg per 10 mg of tumour sample, and the CXCR3alt=ΔCT−16.426758 is:

p=1/(1+exp(−(6.045+10.303(CXCL11)+0.904(CXCR3alt))))

p=1/(1+exp(−(6.045+1.303(arsinh(2.77))+0.904((−16.426758)))))
p=0.0015, therefore a 0.15% probability of being a NAC responder, and is therefore classified a non-responder.

Analogously, a formula for clinical application of a predictive logistic regression model can be developed based on the value of the two biomarkers, CXCL11 and CXCR3A:

y=α+β1(CXCL11)+β2(CXCR3A) with

α=9.558

β1=1.547

β2=1.327

### Example 3: BC Patient Classification Using CXCL11 and CXCR3A. CXCR3alt and CXCR3B Expression

The prediction performance can be improved by including a CXCR3 score which captures the opposing function of the CXCRB splice variant as a second variable in the logistic regression model. The CXCR3 score is a linear combination of the ΔCT values of the three splice variants describing a negative correlation of CXCR3B to CXCR3A and CXCR3alt, respectively:

CXCR3score=CXCR3alt+CXCR3A−CXCR3B

y=α+β1(CXCL11)+β2(CXCR3score) with

α=0.765

β1=1.246

β2=0.353

Using the above regression coefficients, the probability of a patient responding favorably to NAC using levels of four markers where unnormalized CXCL11=348.9 pg per 10 mg of tumour sample, CXCR3alt=ΔCT−8.752, CXCR3A=ΔCT−11.12, and CXCR3B ΔCT=−6.039 is:

p=1/(1+exp(−(0.765+1.246(CXCL11)+0.353(CXCR3alt+CXCR3A−CXCR3B))))

p=1/(1+exp(−(0.765+1.246(arsinh(348.9))+0.353((−8.752−11.12+6.039)))))

p=0.983, therefore a 98.3% probability of a favourable response to NAC, and is therefore classified as a NAC responder.

TABLE 1
11 Plex

| | | | | | | | | XCL1/ | | IL-29/ |
|---|---|---|---|---|---|---|---|---|---|---|
| M-CSF | CXCL9/ | CXCL7/ | CXCL6/ | CXCL11/ | CCL14a/ | CCL19/ | CCL20/ | Lymphotac- | | IFN- |
| (51) | MIG | NAP2 | GCP2 | I-TAC | HCC-1 | MIP3β | MIP3a | tin | IL-11 | lamda1 |
| | (47) | (13) | (15) | (19) | (21) | (26) | (28) | (30) | (34) | (36) |

21 Plex

| | | | | | | | | | | |
|---|---|---|---|---|---|---|---|---|---|---|
| | | | | MIP-4/ | | | | | IL-37/ | |
| MPIF-1 | BRAK | CXCL-16 | HCC-4 | PARC | IL-34 | IL-24 | APRIL | IL-35 | IL-1F7 | IL-19 |
| (12) | (14) | (15) | (19) | (20) | (27) | (28) | (29) | (33) | (34) | (36) |

21 Plex

| | | | | | | | | | |
|---|---|---|---|---|---|---|---|---|---|
| | | | | IL-28B/ | | | | | |
| | HMGB1/ | | | IFN- | BAFF/ | IL-14/a- | IL- | IL- | YKL40/ |
| CCL28 | HM G1 | IFNβ | IL-38 | lamda3 | BLyS | Taxilin | 36β | 32a | CHI3L1 |
| (43) | (51) | (52) | (53) | (55) | (56) | (61) | (63) | (64) | (77) |

23 Plex

| | | | | | | | | | | | | |
|---|---|---|---|---|---|---|---|---|---|---|---|---|
| Eotaxin- | | | | | | | | Eotaxin- | | | | |
| 2 | MCP-2 | BCA-1 | MCP-4 | I-309 | IL-16 | TARC | 6Ckine | 3 | LIF | TPO | SCF | TSLP |
| (12) | (13) | (15) | (18) | (19) | (21) | (26) | (28) | (30) | (34) | (36) | (38) | (43) |

23 Plex

| | | | | | | | | | |
|---|---|---|---|---|---|---|---|---|---|
| | | | | | | SDF- | | | |
| IL- | IL- | IL- | IL- | | | 1a + | ENA- | MIP- | IL- |
| 33 | 20 | 21 | 23 | TRAIL | CTACK | β | 78 | 1d | 28A |
| (45) | (51) | (52) | (54) | (56) | (62) | (64) | (66) | (76) | (77) |

38 Plex

| | | | | | | | | | | | | | | | | | | | |
|---|---|---|---|---|---|---|---|---|---|---|---|---|---|---|---|---|---|---|---|
| | Frac- | | | | | | | | | | | | | | | | | | |
| | tal | IFN- | IFN- | IL- | IL- | IL- | IL- | IL- | IL- | IL- | IL- | IP- | MCP- | MCP- | MIP- | FGF- | | TGF- | G- |
| EGF | kine | a2 | γ | 1β | 2 | 4 | 5 | 6 | 8 | 15 | 17A | 10 | 1 | 3 | 1a | 2 | Eotaxin | a | CSF |
| (12) | (21) | (22) | (25) | (46) | (48) | (53) | (55) | (57) | (63) | (37) | (39) | (65) | (67) | (28) | (72) | (13) | (14) | (15) | (18) |

| 38 Plex | | | | | | | | | | | | | | | | | |
|---|---|---|---|---|---|---|---|---|---|---|---|---|---|---|---|---|---|
| Flt- | GM- | | IL- | IL- | | IL- | IL- | | IL- | IL- | IL- | IL- | IL- | MIP- | TNF- | | |
| 3L | CSF | GRO | 10 | 12P40 | MDC | 12P70 | 13 | sCD40L | 1RA | 1a | 9 | 3 | 7 | 1β | a | TNFβ | VEGF |
| (19) | (20) | (26) | (27) | (29) | (30) | (33) | (35) | (38) | (42) | (44) | (45) | (51) | (61) | (73) | (75) | (76) | (78) |

TABLE 2

| Single variable | Threshold | Unit | Specificity | Sensitivity | AUC | AIC score | Brier score |
|---|---|---|---|---|---|---|---|
| CXCR3alt | −11.27 | dCt | 1.00 | 0.89 | 0.98 | 14.738 | 0.08204389 |
| CXCR3A | −11.97 | dCT | 0.91 | 0.89 | 0.94 | 17.345 | 0.1007318 |
| CXCL11 (I-TAC) | 22.40 | pg/10 mg | 0.82 | 1.00 | 0.91 | 17.634 | 0.1097633 |
| CXCL10 (IP-10) | 703.90 | pg/10 mg | 0.91 | 0.67 | 0.75 | 28.585 | 0.2099636 |
| CXCL9 (MIG) | 11317.05 | pg/10 mg | 0.82 | 0.56 | 0.63 | 30.516 | 0.235402 |
| CXCR3B | −8.43 | dCt | 0.64 | 0.67 | 0.62 | 31.035 | 0.2417187 |
| CXCR3bulk (CXCR3A + | −8.07 | dCt | 0.55 | 0.67 | 0.48 | 31.509 | 0.2473072 |
| B + alt) | | | | | | | |

TABLE 3

| Logistic | | | | | |
|---|---|---|---|---|---|
| regression variables | Specificity | Sensitivity | AUC | AIC score | Brier score |
| CXCL11 + CXCR3bulk | 0.73 | 1.00 | 0.97 | 16.073 | 0.07714034 |
| CXCL11 + CXCR3alt | 1.00 | 1.00 | 1.00 | 10.456 | 0.02430952 |
| CXCL11 + CXCR3A | 1.00 | 1.00 | 1.00 | 9.6856 | 0.01736898 |
| CXCL11 + CXCR3B | 0.73 | 1.00 | 0.97 | 14.976 | 0.06616572 |
| CXCL11 + I(CXCR3alt + | 1.00 | 1.00 | 1.00 | 9.9646 | 0.01981414 |
| CXCR3A) | | | | | |
| CXCL11 + I(CXCR3alt + | 1.00 | 1.00 | 1.00 | 9.4717 | 0.01246996 |
| CXCR3A − CXCR3B) | | | | | |
| CXCL11 + I(CXCR3alt − | 1.00 | 1.00 | 1.00 | 10.58 | 0.02334135 |
| CXCR3B) | | | | | |
| CXCL11 + I(CXCR3A − | 1.00 | 1.00 | 1.00 | 9.5782 | 0.01348617 |
| CXCR3B) | | | | | |

dCt—difference in cycle thresholds
AUC—Area under the reciever operating characteristic curve
AIC—Akaike information criterion, model fitting score
Brier score—model fitting score

TABLE 4

| | | | 95% | |
|---|---|---|---|---|
| Survival | | Hazard | confidence | |
| stratification based on | Coefficient | Ratio | interval | p-value |
| CXCL9 | −0.17 | 0.85 | 0.75-0.96 | 0.01 |
| CXCL10 | −0.13 | 0.88 | 0.78-0.98 | 0.02 |
| CXCL11 | −0.13 | 0.88 | 0.79-0.98 | 0.02 |
| CXCR3 | −0.18 | 0.84 | 0.70-1.00 | 0.05 |
| CXCL4 | 0.12 | 1.13 | 0.96-1.33 | 0.17 |

1. A modified CD3⁺ T cell, particularly a CD3+ CD8+ memory T cell for use in treating cancer, expressing a CXCR3 transgene, wherein the transgene encodes a recombinant protein comprising a human CXCR3 variant selected from:
CXCR3A, CXCR3alt+, and/or CXCR3B,
particularly wherein the human CXCR3 variant, or one of the human CXCR3 variants is CXCR3A and/or CXCR3alt.

2. The modified T cell for use according to claim 1, wherein the CXCR3 transgene encodes only the CXCR3 variant CXCR3alt.

3. The modified T cell for use according to claim 1, wherein the CXCR3 transgene encodes only the CXCR3 variant CXCR3A.

4. The modified T cell for use according to claim 1, wherein the CXCR3 transgene encodes only the CXCR3 variants CXCR3alt and CXCR3A.

5. The modified T cell for use according to claim 1, wherein the CXCR3 transgene encodes only the CXCR3 variants CXCR3alt and CXCR3B.

6. The modified T cell for use according to claim 1, wherein the CXCR3 transgene encodes only the CXCR3 variants CXCR3A and CXCR3B.

7. The modified T cell for use according to claim 1, wherein the CXCR3 transgene encodes the CXCR3 variants CXCR3alt, CXCR3Aand CXCR3B.

8. The modified T cell for use according claim 1, wherein the CXCR3 transgene
a. comprises the reverse complement of the premRNA transcript of CXCR3A, CXCR3alt, and/or CXCR3B, particularly a sequence selected from SEQ ID NO 001, SEQ ID NO 002 and/or SEQ ID NO 003, or
b. comprises the reverse complement of the coding mRNA transcript of CXCR3A, CXCR3alt, and/or CXCR3B, particularly a sequence selected from SEQ ID NO 004, SEQ ID NO 005, and/or SEQ ID NO 006, or
c. encodes an amino acid sequence that has at least (≥) 95% sequence identity to the amino acid sequence encoded by SEQ ID NO 001, SEQ ID NO 002, SEQ ID NO 003, SEQ ID NO 004, SEQ ID NO 005 and/or SEQ ID NO 006, and wherein the encoded protein has the same biological activity as the amino acid sequence encoded by SEQ ID NO 001, SEQ ID NO 002, SEQ ID NO 003, SEQ ID NO 004, SEQ ID NO 005 and/or SEQ ID NO 006,
particularly wherein the CXCR3 transgene encodes an amino acid sequence that has ≥96%, ≥97, ≥98 or even ≥99% sequence identity to the amino acid sequence encoded by SEQ ID NO 001, SEQ ID NO 002, SEQ ID NO 003, SEQ ID NO 004, SEQ ID NO 005 and/or SEQ ID NO 006.

9. The modified T cell for use according to claim 1, wherein the expression level of CXCR3A and/or CXCR3alt is higher than the expression level of CXCR3B, particularly wherein the ratio of the expression level of CXCR3A and/or CXCR3alt in comparison to CXCR3B is more than 1.

10. The modified T cell for use according to claim 1, further expressing a chimeric antigen receptor (CAR) comprising
a. a signal peptide,
b. a target specific recognition domain, particularly wherein the target is a tumour-associated surface antigen, a lineage-specific antigen, a tissue-specific surface antigen, or a virus-specific surface antigen,
c. an effector domain comprising a transmembrane region and one or more intracellular signalling,
d. a linker region, connecting domain (b) and domain (c),

11. The modified T cell for use according to claim 1, further expressing a transgenic T cell receptor (TgTCR) protein, wherein the TgTCR recognises a target selected from a tumour-associated surface antigen, a lineage-specific antigen, a tissue-specific surface antigen, or a virus-specific surface antigen,

12. The modified T cell for use according to claim 10, wherein the target specific recognition domain, or the TgTCR recognises a target selected from a transgenic T cell receptor specific for an antigen selected from LMPA, CMVₚₚ₆₅, GD2, L1CAM, Her2, IL13Ra2, EGFRvIII, CD133, mesothelin, CALX, CEACAM5, TAG-72, CEA, COA-1, PSMA, or c-MET.

13. The modified T cell for use according to claim 1, wherein the cell further expresses a CXCR3 ligand transgene comprising a CXCR3 ligand transgene promotor sequence and a recombinant human CXCR3 ligand, and wherein the transgene comprises:
a. the reverse complement of a premRNA transcript of CXCL9, CXCL10, and/or CXCL11, particularly a sequence selected from SEQ ID NO 007, SEQ ID NO 008, SEQ ID NO 009, SEQ ID NO 010 and/or SEQ ID NO 011, or
b. the reverse complement of a coding mRNA transcript of CXCL9, CXCL10, and/or CXCL11, particularly a sequence selected from SEQ ID NO 012, SEQ ID NO 013, SEQ ID NO 014 and/or SEQ ID NO 015, or
c. a nucleic acid sequence encoding an amino acid sequence that has at least (≥) 95% sequence identity to the amino acid sequence encoded by SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015, and wherein the encoded protein has the same biological activity as the amino acid sequence encoded by SEQ ID NO 007, SEQ ID NO 008, SEQ ID NO 009, SEQ ID NO 010, SEQ ID NO 011, SEQ ID NO 012, SEQ ID NO 013, SEQ ID NO 014 and/or SEQ ID NO 015,
particularly wherein the CXCR3 transgene encodes an amino acid sequence that has ≥96%, ≥97, ≥98 or even ≥99% sequence identity to the amino acid sequence encoded by SEQ ID NO 007, SEQ ID NO 008, SEQ ID NO 009, SEQ ID NO 010, SEQ ID NO 011 SEQ ID NO 012, SEQ ID NO 013, SEQ ID NO 014 and/or SEQ ID NO 015.

14. The modified T cell for use according to claim 1, wherein the cancer is a solid cancer such as a squamous cell cancer or adenocarcinoma, more particularly a cancer selected from breast cancer, colorectal cancer, neuroblastoma, sarcoma, bladder cancer, glioblastoma, hepatocellular cancer, pancreatic cancer, renal cancer, gastrointestinal cancer, or prostate cancer.

15. An isolated preparation of immune cells, particularly a preparation of T cells, wherein the isolated preparation of immune cells comprises at least (≥) 50%, particularly 70%, more particularly ≥80%, even more particularly ≥90% immune cells, particularly T cells, expressing one or more human CXCR3 variants selected from CXCR3A, CXCR3alt+, and/or CXCR3B,
wherein the human CXCR3 variant, or one of the human CXCR3 variants is CXCR3A and/or CXCR3alt.

16. The isolated preparation of cells according to claim 15, wherein the cells are derived from a cancer patient sample, particularly a cancer patient sample selected from peripheral blood, tumour tissue and/or tumour draining lymph node tissue.

17. The isolated preparation of cells according to claim 15, comprising at least (≥) 50%, particularly ≥70%, more particularly ≥80% of any one of the modified immune cells as specified in any one of the claims 1 to 14.

18. The isolated preparation of cells according to claim 15, wherein the cells do not express any transgenes.

19. The isolated preparation of cells according to claim 15, wherein within the immune cells expressing a CXCR3 variant, ≥50%, particularly ≥70%, more particularly ≥80% are:
a. CD8⁺ memory cells, particularly CD8⁺CCR7⁺CD45RA⁺CD95⁺ and/or CD8⁺CCR7⁺CD45RA⁻CD95⁺ memory T cells
b. CD4⁺ memory T cells, particularly T helper type I, T-bet⁺CD4⁺ memory T cells,
c. CD4⁺T regulatory (Treg) cells, particularly CD4⁺CD25⁺Treg cells, or
d. NK or NKT cells, particularly CD56⁺NK or NKT cells.

20. The isolated preparation of cells according to claim 15, for use in
a. treating cancer, particularly a solid cancer such as a squamous cell cancer or adenocarcinoma, more particularly a cancer selected from breast cancer, colorectal cancer, neuroblastoma, sarcoma, bladder cancer, glioblastoma, hepatocellular cancer, pancreatic cancer, renal cancer, gastrointestinal cancer, or prostate cancer.


SEQUENCE LISTING (21 sequences)

SEQ ID NO: 1 - DNA, 1615 bp, Homo sapiens
acaagcaccaaagcagaggggcaggcagcacaccacccagcagccagagcaccagcccagccatggtccttgaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggccgtgctgctggtttccaggggccagcggcgcctgcgggccatgcggctggtggtggtggtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttgtgaggccggaatccgggctcccctttcgcccacagtctgacttccccgcattccaggctcctccctccctctgccggctctggctctccccaatatcctcgctcccgggactcactggcagccccagcaccaccaggtctcccgggaagccaccctcccagctctgaggactgcaccattgctgctccttagctgccaagccccatcctgccgcccgaggtggctgcctggagccccactgcccttctcatttggaaactaaaacttcatcttccccaagtgcggggagtacaaggcatggcgtagagggtgctgccccatgaagccacagcccaggcctccagctcagcagtgactgtggccatggtccccaagacctctatatttgctcttttatttttatgtctaaaatcctgcttaaaacttttcaataaacaagatcgtcagga

SEQ ID NO: 2 - DNA, 1859 bp, Homo sapiens
acaagcaccaaagcagaggggcaggcagcacaccacccagcagccagagcaccagcccagccatggtccttgaggggtccctgggccgatgggatcacgcagaagaatgcgagagaagcagcctttgagaagggaagtcactatcccagagcccaggctgagcggatggagttgaggaagtacggccctggaagactggcggggacagttataggaggagctgctcagagtaaatcacagactaaatcagactcaatcacaaaagagttcctgccaggcctttacacagccccttcctccccgttcccgccctcacaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggccgtgctgctggtttccaggggccagcggcgcctgcgggccatgcggctggtggtggtggtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttgtgaggccggaatccgggctcccctttcgcccacagtctgacttccccgcattccaggctcctccctccctctgccggctctggctctccccaatatcctcgctcccgggactcactggcagccccagcaccaccaggtctcccgggaagccaccctcccagctctgaggactgcaccattgctgctccttagctgccaagccccatcctgccgcccgaggtggctgcctggagccccactgcccttctcatttggaaactaaaacttcatcttccccaagtgcggggagtacaaggcatggcgtagagggtgctgccccatgaagccacagcccaggcctccagctcagcagtgactgtggccatggtccccaagacctctatatttgctcttttatttttatgtctaaaatcctgcttaaaacttttcaataaacaagatcgtcagga

SEQ ID NO: 3 - DNA, 1391 bp, Homo sapiens
acaagcaccaaagcagaggggcaggcagcacaccacccagcagccagagcaccagcccagccatggtccttgaggtaagtgctgctgcccagccaggccaggccagaattcgaagtggaaaacccggggcatcaggggaatgccagggctggcccacagcccagggtcaccacagggttgggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggggtcaagttccgggagcggatgtggatgctgctcttgcgcctgggctgccccaaccagagagggctccagaggcagccatcgtcttcccgccgggattcatcctggtctgagacctcagaggcctcctactcgggcttgtgaggccggaatccgggctcccctttcgcccacagtctgacttccccgcattccaggctcctccctccctctgccggctctggctctccccaatatcctcgctcccgggactcactggcagccccagcaccaccaggtctcccgggaagccaccctcccagctctgaggactgcaccattgctgctccttagctgccaagccccatcctgccgcccgaggtggctgcctggagccccactgcccttctcatttggaaactaaaacttcatcttccccaagtgcggggagtacaaggcatggcgtagagggtgctgccccatgaagccacagcccaggcctccagctcagcagtgactgtggccatggtccccaagacctctatatttgctcttttatttttatgtctaaaatcctgcttaaaacttttcaataaacaagatcgtcaggaccaggg

SEQ ID NO: 4 - DNA, 1107 bp, Homo sapiens
atggtccttgaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggccgtgctgctggtttccaggggccagcggcgcctgcgggccatgcggctggtggtggtggtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttgtga

SEQ ID NO: 5 - DNA, 1248 bp, Homo sapiens
atggagttgaggaagtacggccctggaagactggcggggacagttataggaggagctgctcagagtaaatcacagactaaatcagactcaatcacaaaagagttcctgccaggcctttacacagccccttcctccccgttcccgccctcacaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggccgtgctgctggtttccaggggccagcggcgcctgcgggccatgcggctggtggtggtggtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttgtga

SEQ ID NO: 6 - DNA, 837 bp, Homo sapiens
atgccagggctggcccacagcccagggtcaccacagggttgggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggggtcaagttccgggagcggatgtggatgctgctcttgcgcctgggctgccccaaccagagagggctccagaggcagccatcgtcttcccgccgggattcatcctggtctgagacctcagaggcctcctactcgggcttgtgaggccggaatccgggctcccctttcgcccacagtctga

SEQ ID NO: 7 - DNA, 2761 bp, Homo sapiens
aaagaatttctcaggctcaaaatccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcagaaaacagatcttgattttttttctcttgaatgtacccttcaactgttgaatgtttaatagtaaatcttatatgtccttatttactttttagctttctctcaaataaagtgtaacactagttgagataacacatgaaagctctttaaagggtcgatcgggaacaggaaaaaaaacctatggaaaatatgacaacac

SEQ ID NO: 8 - DNA, 1175 bp, Homo sapiens
gagacattcctcaattgcttagacatattctgagcctacagcagaggaacctccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaa

SEQ ID NO: 9 - DNA, 1048 bp, Homo sapiens
gagacattcctcaattgcttagacatattctgagcctacagcagaggaacctccagtctcagcaccatgaatcaaactgccattctgatttgctgccttatctttctgactctaagtggcattcaagtgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaa

SEQ ID NO: 10 - DNA, 1479 bp, Homo sapiens
gttcagcatttctactccttccaagaagagcagcaaagctgaagtagcagcagcagcaccagcagcaacagcaaaaaacaaacatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaaaaatatcaaaacatatgaagtcctggaaaagagcatctgaaaaacctagaacaagtttaactgtgactactgaaatgacaagaattctacagtaggaaactgagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccaacaattaaatggatttctaggaaaagctaccttaagaaaggctggttaccatcggagtttacaaagtgctttcacgttcttacttgttgcattatacattcatgcatttctaggctagagaaccttctagatttgatgcttacaactattctgttgtgactatgagaacatttctgtctctagaagtcatctgtctgtattgatctttatgctatattactatctgtggttacggtggagacattgacattattactggagtcaagcccttataagtcaaaagcatctatgtgtcgtaaaacattcctcaaacattttttcatgcaaatacacacttctttccccaaacatcatgtagcacatcaatatgtagggagacattcttatgcatcatttggtttgttttataaccaattcattaaatgtaattcataaaatgtactatgaaaaaaattatacgctatgggatactggcaaaagtgcacatatttcataaccaaattagtagcaccagtcttaatttgatgtttttcaacttttattcattgagatgttttgaagcaattaggatatgtgtgtttactgtactttttgttttgatccgtttgtataaatgatagcaatatcttggacacatctgaaatacaaaatgtttttgtctaccaaagaaaaatgttgaaaaataagcaaatgtatacctagcaatcacttttactttttgtaattctgtctcttagaaaaatacataatctaatcaatttctttgttcatgcctatatactgtaaaatttaggtatactcaagactagtttaaagaatcaaagtcatttttttctctaataaactaccacaacctttcttttttaaaaaaa

SEQ ID NO: 11 - DNA, 1475 bp, Homo sapiens
gttcagcatttctactccttccaagaagagcagcaaagctgaagtagcagcagcagcaccagcagcaacagcaaaaaacaaacatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaattgaaagaaagaatttttaaaaatatcaaaacatatgaagtcctggaaaagagcatctgaaaaacctagaacaagtttaactgtgactactgaaatgacaagaattctacagtaggaaactgagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccaacaattaaatggatttctaggaaaagctaccttaagaaaggctggttaccatcggagtttacaaagtgctttcacgttcttacttgttgcattatacattcatgcatttctaggctagagaaccttctagatttgatgcttacaactattctgttgtgactatgagaacatttctgtctctagaagtcatctgtctgtattgatctttatgctatattactatctgtggttacggtggagacattgacattattactggagtcaagcccttataagtcaaaagcatctatgtgtcgtaaaacattcctcaaacattttttcatgcaaatacacacttctttccccaaacatcatgtagcacatcaatatgtagggagacattcttatgcatcatttggtttgttttataaccaattcattaaatgtaattcataaaatgtactatgaaaaaaattatacgctatgggatactggcaaaagtgcacatatttcataaccaaattagtagcaccagtcttaatttgatgtttttcaacttttattcattgagatgttttgaagcaattaggatatgtgtgtttactgtactttttgttttgatccgtttgtataaatgatagcaatatcttggacacatctgaaatacaaaatgtttttgtctaccaaagaaaaatgttgaaaaataagcaaatgtatacctagcaatcacttttactttttgtaattctgtctcttagaaaaatacataatctaatcaatttctttgttcatgcctatatactgtaaaatttaggtatactcaagactagtttaaagaatcaaagtcatttttttctctaataaactaccacaacctttcttttttaaaaaaa

SEQ ID NO: 12 - DNA, 378 bp, Homo sapiens
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataa

SEQ ID NO: 13 - DNA, 297 bp, Homo sapiens
atgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaa

SEQ ID NO: 14 - DNA, 285 bp, Homo sapiens
atgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaa

SEQ ID NO: 15 - DNA, 321 bp, Homo sapiens
atgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaattgaaagaaagaatttttaaaaatatcaaaacatatgaagtcctggaaaagagcatctga

SEQ ID NO: 16 - DNA, 19 bp, Artificial Sequence
  Probe
tcactatcccagagcccag

SEQ ID NO: 17 - DNA, 25 bp, Artificial Sequence
  Primer
ccgtacttcctcaactccatccgct

SEQ ID NO: 18 - DNA, 19 bp, Artificial Sequence
  Primer
tcctataactgtccccgcc

SEQ ID NO: 19 - DNA, 24 bp, Artificial Sequence
  Probe
ccggaacttgacccctgtgggaag

SEQ ID NO: 20 - DNA, 17 bp, Artificial Sequence
  Primer
cacgacgagcgcctcaa

SEQ ID NO: 21 - DNA, 16 bp, Artificial Sequence
  Primer
gttggggcagcccagg